(19) United States
(12) Patent Application Publication
(10) Pub. No.: US 2009/0232843 A1
(43) Pub. Date: Sep. 17, 2009

(54) Identifying and predicting influenza variants and uses thereof

(76) Inventor: Henry L. Niman, Pittsburgh, PA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/006,795

(22) Filed: Jan. 4, 2008

Related U.S. Application Data
(63) Continuation of application No. PCT/US2006/026354, filed on Jul. 7, 2006.
(60) Provisional application No. 60/697,770, filed on Jul. 8, 2005.
(60) Provisional application No. 60/703,779, filed on Jul. 29, 2005.
(60) Provisional application No. 60/774,922, filed on Feb. 16, 2006.

(51) Int. Cl. A61K 39/145; G01N 33/53; C12Q 1/70; G01N 33/566; C07H 21/04; A61K 31/7088; A61P 31/16

(52) U.S. Cl. 424/209.1; 436/94; 435/5; 436/501; 536/23.72; 514/44 R

(57) ABSTRACT

The instant invention provides methods for determining, predicting and characterizing the genetic variability of viruses, in particular, influenza. Accordingly, the invention provides methods for identifying virulent pathogens, genetic mutations within pathogens that are relevant to animal health, and methods and compositions for prophylactic or therapeutic intervention against such pathogens.

### RELATED APPLICATIONS

This application is related to U.S. Provisional Application No. 60/697,770 filed on Jul. 8, 2005 (“Copy Choice Recombination and Uses Thereof”), U.S. Provisional Application No. 60/703,779, filed on Jul. 29, 2005 (“Identifying Genetic Transfer Events and Uses Thereof”), and U.S. Provisional Application No. 60/774,922 filed on Feb. 16, 2006 (“Identifying and Predicting Genetic Transfer Events and Uses Thereof”), the entire contents of which are incorporated herein by reference.

The contents of all references, patents and published patent applications cited throughout this application are hereby incorporated herein by reference in their entirety.

### BACKGROUND

Despite remarkable achievements in the development of molecular genetics for understanding human and animal disease as well as determining the genetic nature of pathogens, rules for prediction or prognosis of future disease and pathogen virulence remain elusive. Typically, genetic alterations in cell genomes resulting in disease (or disease susceptibility) or genetic sequence of virulent pathogens is collected a posteriori, cataloged, and then used to make a diagnosis, and determine an appropriate therapeutic. Accordingly, responding therapeutically to the sequelae of genome instability in a subject suffering from a genetic disease (or disease susceptibility) and/or pathogen is typically reactive rather than proactive. This is especially true in treating, for example, human and animal response to pathogens such as viruses.

Development of viral vaccines presents unique challenges to modern medicine (see, for example, Ault, A. (2004) Science, 303:1280). Due to the constant evolution of many pathogens, and in particular, viruses, development of effective vaccines is often a difficult, and imperfect, process. Reliable prediction of the future molecular evolution of viral genomes would be expected to advance humankind's ability to combat such pathogens both prophylactically and therapeutically.

Viruses are the smallest of parasites, and are completely dependent upon the cells they infect for their reproduction. Of the viruses that infect humans, many infect their hosts without producing overt symptoms, while others (e.g., influenza A) produce a well-characterized set of symptoms. Importantly, although symptoms can vary with the virulence of the infecting strain, identical viral strains can have drastically different effects depending upon the health and immune response of the host.

A better understanding of the molecular events that lead to genome instability not only for understanding human and animal disease but also the evolution of pathogens such as viruses, is needed. Indeed, the ability to predict the molecular evolution of pathogenic genomes would be broadly expected to enhance the design of anti-pathogenic agents.

### SUMMARY OF THE INVENTION

The instant invention is based at least in part on the discovery that genome instability across a wide array of organisms, including eukaryotic cells, prokaryotic cells, and viruses occurs as a function of a newly-identified mechanism termed copy-choice recombination. Heretofore, random mutations, gene translocations, and/or gene reassortment were thought to be the predominant mechanisms of viral gene evolution. Indeed, until recently, it was believed that viral evolution has been primarily due to the accumulation of small mutations in the viral genome. However, this mechanism explains only a small part of the evolution of viruses.

The newly-identified mechanism described herein can account for the acquisition of gene mutations between two or more gene sequences in a cellular or organismal context. Accordingly, the mechanism disclosed herein is predictive for mutations that can occur in multicellular organisms, eukaryotic cells, prokaryotic cells, in pathogens and microbes, and, in particular, viruses.

Without being bound by theory, the invention provides a mechanism of genetic evolution based upon recombination or acquisition of a previously existing sequence(s) by gene copy recombination, i.e., referred to herein as copy choice recombination rather than through the introduction of de novo genetic mutation(s) based on, e.g., polymerase proof-reading errors, spontaneous point mutations, and the like. Because an exchange of genetic information occurs between a section of genetic sequence of one virus and/or cell and another virus and/or cell, the exchange can be referred to as a genetic transfer event. The observation that intergenic and/or intragenic genetic exchange occurs between pathogens (e.g., viruses, bacteriophage) and cells (e.g., prokaryotic or eukaryotic host cells) allows for the prediction of genetic transfer events.

This mechanism of genetic change can be readily exploited to provide predictive rules by which genetic changes in the genomes of eukaryotic cells, prokaryotic cells, pathogens, microbes, viruses, and the like can be forecast. Accordingly, the likelihood of a genetic alteration appearing in a given genome allows for a priori intervention, e.g., the prediction or prognosis of genetic disease or disorder, or emergence or appearance of a strain of pathogen, e.g., a virulent strain, such that therapy can be rationally designed.

The predictive rules of the invention, i.e., of copy-choice recombination include, e.g., 1) that the prediction that genetic alterations, e.g., genetic transfer events, are acquired in tracts that resemble the haplotypes that can be found in higher eukaryotic genomic sequence, 2) that the prediction that genetic alterations typically comprise a high frequency of nucleic acid base transitions, and/or 3) that the prediction that genetic alterations are acquired from an existing gene sequence(s) from a parental nucleic acid sequence.

In one aspect, the predicative rules of the invention can be used to improve human or animal health by forecasting the likelihood of a disease or disorder or the pharmacogenomic responsiveness of a subject.

In another aspect, the predicative rules of the invention can be used to improve human or animal health by forecasting the likelihood of the appearance or emergence of a pathogen, for example, a virulent strain of virus, thereby allowing for therapeutic intervention, for example, administering of an anti-pathogenic agent, for example, an antiviral and/or vaccine (e.g. passive or active vaccine).

In certain aspects, the rules of the invention are applied to predict the time, site and composition of specific progeny viral strains that will arise from parental viral strains. Such predictions involve anticipation of genetic transfer events deemed to be of special interest, e.g., transfer events involving mutant sequences correlated with a molecular, clinical or pathological characteristic of at least one strain of parental virus. Having identified the presence of a mutant sequence correlated with a molecular, clinical or pathological characteristic in at least one parental strain of virus, the methods of the invention are used to predict the time and place of emergence of progeny viral strain sequences arising from a genetic transfer event comprising replacement of one parental viral strain sequence that lacking the identified mutant sequence with the mutant sequence of the parental viral strain identified to contain the mutant sequence.

In one aspect, the invention relates to a method of predicting progeny viral strain sequence from sequences of a first parental viral strain and a second parental viral strain, comprising identifying a first parental viral strain sequence comprising one or more sequences correlated with a characteristic of the virus; identifying a second parental viral strain sequence lacking one or more of the one or more sequences of the first parental viral strain; and predicting progeny viral strain sequences capable of arising from a genetic transfer event comprising replacement of a second parental viral strain sequence with a first parental viral strain sequence.

In certain embodiments, the viral strains are influenza viruses. In one embodiment, the characteristic is genotypic, phenotypic, molecular, epidemiological, clinical, or pathological. In a related embodiment, the molecular characteristic is a nucleic acid alteration or amino acid alteration. In certain embodiments, the nucleic acid or amino acid alteration is in an influenza sequence selected from the group consisting of HA, NA, NP, NA, PA, PB1, PB2, M1, M2, NS1, and NS2, or combinations thereof. In specific embodiments, the nucleic acid or amino acid alteration is in an influenza sequence selected from the group consisting of HA, NA, NP, NA, PA, PB1, PB2, M1, M2, NS1, and NS2, or combinations thereof, as set forth in any of the Tables herein.

In one embodiment, the nucleic acid or amino acid alteration is in an influenza HA sequence. In another embodiment, the nucleic acid or amino acid alteration is in an influenza HA sequence as set forth in any of the Tables herein. In certain embodiments, the alteration is in an influenza HA sequence at a residue position(s) selected from the group consisting of 190, 225, 226, 227, 228, and combinations thereof.

In another embodiment, the nucleic acid or amino acid alteration is in an influenza NA sequence. In certain embodiments, the nucleic acid or amino acid alteration is in an influenza NA sequence as set forth in any of the Tables herein.

In an additional embodiment, the molecular characteristic is selected from the group consisting of viral infectivity, viral antigenicity, viral replication, and viral binding to a host cell receptor. In certain embodiments, the binding of the first parental viral strain to a cellular receptor is altered, as compared to the binding of the second parental viral strain to the cellular receptor. In specific embodiments, the binding is determined using a glycan chip assay. In another embodiment, the host cell receptor is an α2-6-linked sialic acid glycoprotein.

In a further embodiment, the first parental viral strain sequence infects a host animal of a population of a first geographic range and the second parental viral strain sequence infects a host animal of a population of a second geographic range. In one embodiment, at least one of the first or second parental viral strain sequences is isolated from a host animal. In certain embodiments, the first and second geographic ranges do not overlap. In another embodiment, the host animals of the first and second parental viral strains are of different species. In one embodiment, at least one of the host animals of the first or second parental viral strains is a migratory bird. In a related embodiment, at least one of the host animals of the first or second parental viral strains is a migratory bird with a geographic range selected from the group consisting of North Africa, Europe, Asia, Middle East, Near East, North America, South America, and combinations thereof. In an additional embodiment, at least one of the host animals is avian. In certain embodiments, the animal is selected from the group consisting of a duck, chicken, turkey, ostrich, quail, swan, and goose. In additional embodiments, at least one of the host animals is selected from the group consisting of swine, chicken, duck, sheep, cattle, goat, and human. In one embodiment, at least one of the host animals is swine.

In certain embodiments, the first and second geographic ranges are projected to overlap within a time span selected from the group consisting of about a day, about a week, about 1 month, about 2 months, about 3 months, about 5 months, about 7 months, about 9 months, about 12 months, and ranges or intervals thereof. In one embodiment, the first and second parental viral strains are not predicted to have occupied the same geographic range. In an additional embodiment, the first and second geographic ranges are newly-overlapping. In further embodiments, the influenza is selected from the group consisting of influenza A, influenza B, and influenza C. In certain embodiments, the acceptor viral strain is selected from the group consisting of influenza A, influenza B and influenza C. In another embodiment, the genetic transfer event is a recombination-mediated genetic transfer event. In an additional embodiment, the genetic transfer event is occurs or is identified from cells cultured in vitro with one or more viral strains. In certain embodiments, the genetic transfer event involves a non-genomic DNA or RNA intermediate.

In another embodiment, the length of the first parental viral strain sequence is selected from the group consisting of about 5-10 nucleotides, about 10-20 nucleotides, about 10-20 nucleotides, about 20-50 nucleotides, about 50-100 nucleotides, about 100-1000 nucleotides, about 10-20 nucleotides, about 10-20 nucleotides, and ranges or intervals thereof. In certain embodiments, the first sequence and second sequence are at least 30% identical, at least 40% identical, at least 50% identical, at least 70% identical, at least 80% identical, at least 90% identical, at least 95%, at least 95%, at least 97%, at least 99% or ranges or intervals thereof. In an additional embodiment, the method further comprises producing a therapeutic compound or vaccine to at least one progeny viral strain. In another embodiment, the method further comprises administration of the therapeutic compound or vaccine to a subject.

In an additional embodiment, the invention relates to a sequence identified according to any of the methods of the invention that is suitable for use in the development of a prognostic compound, diagnostic compound, therapeutic compound, or vaccine. In certain embodiments, the sequence comprises one or more sequences as set forth in any of the Tables herein.

In another embodiment, the invention relates to a composition comprising a nucleic acid or polypeptide sequence identified according to the methods of the invention.

A further aspect of the invention relates to a composition comprising an influenza nucleic acid or polypeptide sequence having an alteration as set forth in any of the Tables herein. In one embodiment, the nucleic acid or polypeptide sequence is an altered influenza HA sequence. In another embodiment, the nucleic acid or polypeptide sequence is an altered influenza NA sequence. In certain embodiments, the influenza HA sequence comprises an alteration at a residue position(s) selected from the group consisting of 190, 225, 226, 227, 228, and combinations thereof. In an additional embodiment, the nucleic acid or polypeptide sequence comprises an alteration in an influenza NA sequence.

Another embodiment of the invention relates to a vaccine composition comprising an altered influenza nucleic acid or polypeptide sequence according to any of the methods or compositions of the invention. An additional embodiment relates to a method of immunizing an animal or human subject against influenza comprising administering such a vaccine composition to the subject.

A further aspect of the invention relates to a kit for predicting or identifying the occurrence of an influenza virus strain comprising an influenza sequence or influenza composition as set forth in any of the Tables herein.

In another embodiment, the invention provides for a comparison of parental viral strains with their mutant progeny viral strains which can be used to define and elucidate selective pressures on rapid evolution. The identification of recombinants can be used to identify genetic instability, which is currently evident in many viruses throughout the world, for example, influenza A and influenza B. The parental viruses can also be used to create recombinants prior to detection in field isolates and such recombinants can be used to make protective vaccines against future recombinants, which cause significant disruptions in animal husbandry and human health.

In still another embodiment, the invention provides rules that can be applied, e.g., to predict the genetic composition and, optionally, associated phenotypic traits (e.g., drug resistance) of viruses or bacteriae that arise from the mixing within a single host organism of distinct “parental” viruses or bacteriae (e.g., ebola, flu and/or HIV; foot and mouth and Newcastle disease; SARS, HIV and/or astroviruses; HIV and coronavirus; distinct drug-resistant bacterial strains, etc.).

In yet another embodiment, the invention provides methods of generating libraries of diverse viral sequences to be used, for example, in the manufacture of viral vaccines, or for testing of antiviral compounds. The invention further provides methods of identifying parental viral strains.

A further aspect of the instant invention features an influenza sequence comprising the sequence TGAAAGAACTT, suitable for use in the development of a prognostic compound, diagnostic compound, therapeutic compound, or vaccine.

The instant invention also provides methods for monitoring the efficacy of viral vaccines and for monitoring the diversity of a viral population.

Accordingly, the invention has several advantages, which include, but are not limited to, the following:
providing rules for determining the nature of genetic alteration occurring or likely to occur in eukaryotes, prokaryotes, and pathogens and predicting future genetic alterations;
providing methods for prognosis of genetic alterations in a human or animal and/or pharmacogenomic responsiveness of a human or animal;
providing methods for determining gene sequences in a human or animal suitable for modulating thereby preventing or treating a disease or disorder;
methods for identifying rational candidate pathogens for therapeutic intervention;
methods and compositions relating to the development of therapeutics against pathogen targets, for example, viral pathogens; and
methods and compositions relating to the development of therapeutics against pathogens, for example, viral pathogens, having acquired or susceptible for acquiring a genetic transfer event from another pathogen, for example, viral pathogen, and/or host cell.

Other features and advantages of the invention will be apparent from the following detailed description and claims.

### BRIEF DESCRIPTION OF THE DRAWINGS

FIG. 1 depicts major flyways of migratory birds in relation to the global spread of the H5N1 influenza strain.

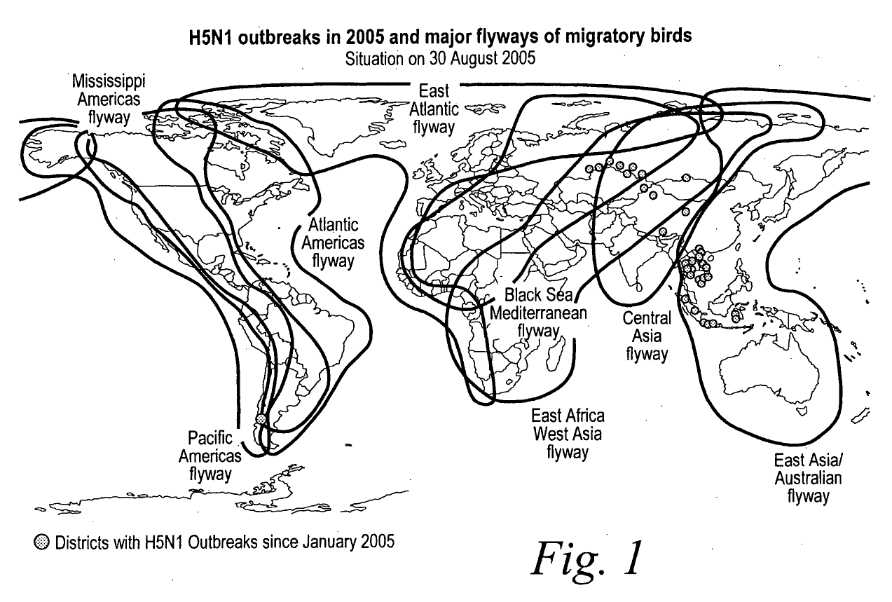

FIG. 2 shows regions of sequence identity in the PB2 gene of Canadian swine influenza isolates SW/ON/11112 (11112), SW/ON/23866 (23866), SW/ON/57561 (57561), SW/AB/56626 (56626), SW/ON/48235 (48235), SW/ON/55383 (55383), SW/ON/53518 (53518) with SW/TN/24/77 (H1N1), SW/NC/35922/98(H3N2), SW/KO/CY02/02(H1N2), SW/ON/112/04(H1N1), SW/ON/48235/04(H1N2) and SW/ON/53518(H1N1).

FIG. 3 shows regions of sequence identity in the PA gene of Canadian swine influenza isolates of FIG. 2 with SW/TN/26/77(H1N1), SW/A/1973/31(H1N1), SW/ON/1112/04(H1N1), SW/ON/55383/04 and SW/53518/03(H1N1).

FIG. 4 shows regions of sequence identity in the PB1 gene of Canadian swine influenza isolates SW/ON/23866 (23866), SW/ON/11112 (11112), SW/ON/53518 (53518), SW/ON/48235/04(H1N2) and SW/ON/55383 (55383) with SW/ON/23866/04(H1N1), SW/ON/41848/97(H3N2), FU/114/96(H3N2), SW/ON/48235/04(H1N2), HK/498/97(H3N2), SW/IN/9K035/99(H1N2) and NY/58/2003(H3N2).

FIG. 5 shows regions of sequence identity in the HA gene of Canadian swine influenza isolates SW/ON/23866 (23866), SW/ON/53518 (53518), SW/ON/11112 (11112), SW/ON/57561 (57561) and SW/AB/56626 (56626) with SH/106/91(H1N1), WI/4755/94(H1N1), NE/1/92(H1N1), SW/ON/23866/04(H1N1) and SW/ON/57561/03(H1N1).

FIG. 6 shows regions of sequence identity in the NP gene of Canadian swine influenza isolates SW/ON/23866 (23866), SW/ON/11112 (11112), SW/ON/57561 (57561), SW/ON/53518 (53518), SW/ON/48235/04(H1N2), SW/ON/55383 (55383), SW/AB/56626 (56626) with SW/IA/930/01(H1N2), SW/ON/23866/04(H1N1), SW/ON/57561/03(H1N1) and SW/ON/48235/04(H1N2).

FIG. 7 shows regions of sequence identity in the NA gene of Canadian swine influenza isolates SW/ON/11112 (11112), SW/ON/57561 (57561), SW/ON/53518 (53518), SW/ON/23866 (23866) and SW/AB/56626 (56626) with SW/ON/11112/04(H1N1), SW/ON/57561/03(H1N1) and WI/4754/94(H1N1).

FIG. 8 shows regions of sequence identity in the MP gene of Canadian swine influenza isolates SW/ON/48235/04(H1N2), SW/ON/55383 (55383), SW/ON/23866 (23866), SW/ON/11112 (11112), SW/ON/53518 (53518), SW/ON/57561 (57561) and SW/AB/56626 (56626) with SW/NC/35922/98(H3N2), SW/ON/2/81(H1N1), SW/WI/3523/88(H1N1), SW/ON/48235/04/(H1N2), SW/ON/11112/04(H1N1) and SW/ON/57556/1/03(H1N1).

FIG. 9 shows regions of sequence identity in the NS gene of Canadian swine influenza isolates SW/ON/23866 (23866), SW/ON/57561/03(H1N1), SW/ON/11112 (11112), SW/ON/48235/04(H1N2), SW/ON/55383 (55383), SW/ON/53518 (53518) and SW/AB/56626 (56626) with SW/ON/57561/03(H1N1), SW/ON/48235/04(H1N1) and SW/AB/56626/03(H1N1).

FIG. 10 shows regions of sequence identity in the HA gene of Chinese swine influenza isolates with Swine/Fujian/F1/2001(H5N1), Swine/Guangdong/4/2003(H5N1), Crow/Osaka/102/2004(H5N1), Tree Sparrow/Henan/2/2004(H5N1) and Duck/Hong Kong/2986.1/2000(H5N1).

FIG. 11 shows regions of sequence identity in the PA gene of Chinese swine influenza isolates with Duck/Guangxi/50/2000(H5N1), Migratory Duck/Jiangxi/2300/2005(H5N1), Swine/Guangdong/4/2003(H5N1) and Swine/Guangdong/1/2003(H5N1).

### DETAILED DESCRIPTION

In order to provide a clear understanding of the specification and claims, the following definitions are conveniently provided below.

### DEFINITIONS

The term, “parental viral strains” is intended to mean the two, or more, viral strains in a population that supply the genetic material to the mutant progeny viral strains in the population through a copy choice recombination mechanism. The parental viral strains are two or more strains of virus that are present in a recently (e.g., within one, two, three, six, twelve, or more months) isolated population of viruses. In one aspect the parental viral strains are the most prevalent sequences in a population. In another aspect, the parental viral strains are the most diverse sequences in a population.

The term, “mutant progeny viral strains” as used herein is intended to mean the viral progeny derived from the parental viral strains. In one embodiment, the mutant progeny viral strains are created by a copy-choice recombination mechanism using the genetic material provided by the parental strains. In one embodiment, the mutant progeny viral strains are isolated from a population of viruses based on one or more desired criteria, e.g., nucleotide sequence, polypeptide sequence, virulence, host range, or tropism.

Similarly, the term “parental bacteria strains” is intended to mean the two, or more, bacteria strains in a population that supply the genetic material to the mutant progeny bacteria strains in the population through a copy choice recombination mechanism. The parental bacteria strains are two or more strains of bacteria that are present in a recently (e.g., within one, two, three, six, twelve, or more months) isolated population of bacteria. In one aspect the parental bacteria strains are the most prevalent sequences in a population. In another aspect, the parental bacteria strains are the most diverse sequences in a population.

Likewise, the term “mutant progeny bacteria strains” as used herein is intended to mean the bacteria progeny derived from the parental bacteria strains. In one embodiment, the mutant progeny bacteria strains are created by a copy-choice recombination mechanism using the genetic material provided by the parental strains. In one embodiment, the mutant progeny bacteria strains are isolated from a population of bacteria based on one or more desired criteria, e.g., nucleotide sequence, polypeptide sequence, drug resistance, pathogenicity, infectivity, etc.

The term “copy choice recombination” as used herein is intended to mean the mechanism of viral or bacterial recombination in which a progeny viral or bacterial strain is made in a cell or organism that has been infected by two or more parent viral strains and the genetic material of the progeny is a mix of the genetic material of the parent strains. Without being bound by mechanism, copy choice mechanism results from the DNA or RNA replication machinery starting on DNA or RNA from one parent and switching to the DNA or RNA from a second parental strain during duplication of a piece of DNA or RNA. This process can happen one or more times thereby resulting in progeny virus or bacteria that has a DNA or RNA sequence that is a mix of the two parental strains.

Sequences produced by copy-choice recombination, e.g., progeny sequences, can contain any number of nucleotide changes, including one or more nucleotide changes as compared with parental sequences, e.g., 2-5, 5-10, 10-20, 20-50, 50-100, 100-500, 500 or greater changes, typically by recombination, e.g. copy-choice recombination, occurring within a given length of nucleic acid, between two or more strands of nucleic acid, e.g., within two nucleotides or more, e.g., 3-5, 5-10, 10-100, 100-1 kb, 1 kb-10 kb, 10 kb or more, or any range or interval thereof.

The term “transition/transversion ratio” as used herein is intended to denote a ratio between the number of times a given sequence has a transition, e.g., the substitution of a purine for a purine, or a pyrimidine for a pyrimidine, versus the number of times the sequence has a transversion, e.g., a purine for a pyrimidine or a pyrimidine for a purine. One would expect the ratio to be 0.5 if it were a random process. However, looking at multiple data sets, it has been determined that the ration is often 2 or higher, indicative that the process is not random and that transitions are favored over transversions (see the Exemplification).

The term “genetic transfer event” refers to an exchange of sequence information between two or more gene loci. Such sequence transfer may be inter- or intragenic, in cis or in trans, and/or between one or more species of pathogens (e.g., viral pathogens) and/or cells (e.g., host cells or organisms).

The present invention, at least in part, is based on the surprising observation that recombination, rather than de novo mutation, is a driving force of viral evolution. In particular, the present invention, at least in part, is based on the observation that pathogens can exchange nucleic acid sequence intergenically or intragenically between one or more pathogens and/or cells, e.g., host cells, with which the pathogens can reside (or infect and/or co-infect). In one observation, progeny strains of influenza are effectively derived as haplotypes from divergent, “parental” strains of influenza A and/or influenza B, revealing that dual infections of a single cell or organism with two or more distinct strains of virus (or distinct types of virus, e.g., influenza and HIV, or distinct strains of bacteria) can accelerate viral evolution. The present invention therefore provides rules for predicting the outcome of such real-world or controlled mixing experiments. In certain aspects of the invention, these rules can be applied to predict progeny influenza A and/or influenza B strains that represent optimal vaccine targets, based upon knowledge (optionally real-time knowledge) of the genetic makeup of the prevalent influenza A and/or influenza B strains in a population. In other observations, other viral pathogens are identified as having acquired a genetic transfer event.

In one aspect, the rules of the invention may be applied to enable prediction of the genomic composition and/or phenotypic traits e.g., drug resistance, of progeny viral strains derived from at least two parental strains of virus. Such progeny virus can then be used, e.g., in subsequent drug screening and/or vaccine development steps.

In one aspect, the instant invention provides a method for identifying parental viral strains in a population of viruses, wherein the population comprises parental viral strains and mutant progeny viral strains, comprising the steps of: obtaining the nucleic acid or polypeptide sequence of one or more viral genes from a number of isolated viral strains from the population, the number sufficient to allow for identification of the viral strains most prevalent in the population, the viral strains having the greatest sequence divergence in the population, or both; identifying the viral strains most prevalent in the population, or viral strains with the greatest sequence divergence in the population, or both; wherein the most prevalent viral sequences, or the viral sequences with the greatest divergence are the parental viral strains.

In one embodiment, the parental viral strains are the two most prevalent sequences in the population. In another embodiment, the parental strains are the two strains with greatest sequence divergence.

In one embodiment, the viruses used in the methods of the invention are from a period of time sufficient to allow for the determination of the parental and mutant progeny viral strains. For example, the period of time in which isolated viruses can be used in the methods of the invention can be 1 month, 2 months, 3 months, 4 months, 6 months, 1 year, 2 years, 3 years, 4 years, 5 years, or more. In one exemplary embodiment, the viruses used in the methods of the invention are from one outbreak season, e.g., one influenza season.

In another embodiment, the methods of the invention use viruses from a defined geographic area, e.g., one in which infected hosts have reasonable chance of interacting. For example, defined geographic areas are southeast Asia, or the continental United States.

In a related embodiment, the most prevalent viral sequences, or the viral sequences with the greatest sequence divergence are determined by aligning multiple nucleic acid or polypeptide sequences. In another related embodiment, the mutant progeny viral strains are formed by recombination according to a copy-choice mechanism.

In another embodiment, the viral sequence has acquired a genetic transfer event from another virus (e.g., strain or species) and/or host cell within which it can reside or infect.

Sequence alignments can be done using, for example, a mathematical algorithm. In one embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

Further, algorithms based upon sequence alignment programs can be developed that automatically compare viral sequences deposited in a database to determine the sequence identity in a population. Bioinformatic approaches can be used to monitor the amount of sequence diversity as a function of time, and location, thereby alerting medical professionals as to when their intervention, i.e., immunization, efforts should be increased. A Bioinformatics approach would be particularly useful for viral populations where there are large databases that would be difficult to align and/or sort, e.g., by date or location, manually, e.g., HIV or influenza A and/or influenza B. Bioinformatics can be used to determine the parental viral strains in a population of viruses and/or determine the mutant viral progeny viruses in a population of viruses by sorting the nucleic acid or polypeptide sequences by, for example, the number and/or location of non-identical nucleotides or amino acids, respectively.

In another aspect, bioinformatics can be used to evaluate databases of viral sequences to identify historically significant sequence variations in a viral gene sequence. The emergence of a previously identified sequence polymorphism is indicative of copy-choice recombination. Without being bound by mechanism, the emergence of a sequence polymorphism in a population of viruses that has not been observed for some time is a sign that there has been copy-choice recombination between two viruses. This approach will allow one of skill in the art to identify, in silico, mutant progeny viral strains that may be problematic, e.g., have high infectivity. Further, analysis of viral sequences in a database for the presence of a known sequence polymorphism that is normally not found in a given geographic area can indicate that copy-choice recombination has occurred.

In one aspect, the methods of the invention may use a computer based program to identify multiple cross-over points in mutant progeny viral strains. Due to the high number of cross-over points in some genes formed by copy choice recombination (often 10-100 cross-over points per gene) computer algorithms will be useful tools to determine the precise location of cross-over points. These computer algorithms can compare a large database of viral sequences to determine the location of cross-overs in a parental viral strain that gave rise to mutant progeny viral strains. The precise mapping of these locations in combination with analysis of the various polymorphisms will allow one of skill in the art to classify viruses based on genotype rather than the serotype classification currently used.

### Computer Prediction Methods

The identification of influenza progeny strains of the present invention can also be conducted with the benefit of structural or modeling information concerning the sequences to be generated, such that the potential for generating progeny strains of importance for diagnostics and/or vaccine development is increased. The structural or modeling information can also be used to guide the selection of predetermined sequences to introduce into defined regions. Still further, actual results obtained with the present selection methods of the invention can guide the selection (or exclusion) of subsequent progeny sequences to be identified, made and/or screened in an iterative manner. Accordingly, structural or modeling information can be used to generate initial subsets of progeny sequences for use in the invention as parental strains for future generations, thereby further increasing the efficiency of predicting progeny sequences.

In a particular embodiment, in silico modeling is used to eliminate the production of any sequence predicted to have poor or undesired structure and/or function. In this way, the number of progeny sequences identified and/or produced can be reduced, thereby increasing signal-to-noise in the progeny sequence output of the invention, optionally used in subsequent iterations of the methods of the invention. In another particular embodiment, the in silico modeling is continually updated with additional modeling information, from any relevant source, e.g., from gene databases (e.g., NCBI, Genbank, influenza sequence databases, etc.) and protein sequence and three-dimensional databases and/or results from previously tested sequences, so that the in silico database becomes more precise in its predictive ability. Accordingly, the methods of the invention may be run as, e.g., a macro capable of leveraging the sequence content of art-recognized sequence databases containing influenza sequence. Such a macro and/or computer-assisted program may be iteratively updated as additional sequences are deposited in sequence databases. In fact, as influenza databases continue to expand in content, the value of information produced via practice of the methods of the present invention is anticipated to rise.

In a preferred embodiment, one or more of the above steps are computer-assisted. The method is also amenable to be carried out, in part or in whole, by a device, e.g., a computer driven device. Accordingly, instructions for carrying out the method, in part or in whole, can be conferred to a medium suitable for use in an electronic device for carrying out the instructions. In sum, the methods of the invention are amendable to a high throughput approach comprising software (e.g., computer-readable instructions) and hardware (e.g., computers, robotics, and chips).

In one embodiment, mutant progeny viral strains can be produced by a copy-choice recombination mechanism in combination with reassortment. In another embodiment, the mutant viral progeny viruses are produced by copy-choice recombination in the absence of reassortment.

Further, in vitro or in vivo techniques can be used to selectively recombine individual genes from different viruses in the population to produce mutant viral progeny viruses. For example, a number of genes from a population of viruses can be analyzed using, for example, sequence alignments. One of skill in the art can isolate genes with desired sequences from the population and use those genes to infect a host cell, egg, or animal to produce a desired set of recombinants. In this situation the genes used to infect the host can come from multiple different viruses (e.g., 3, 4, 5, 6, 7, 8, 9, 10, 11, 12, 13, 14, 15 or more different viruses).

In one aspect of the invention, the methods of the invention can be used with any viruses that infect a subject. The term “subject” is intended to include organisms which are capable of having a viral infection. Examples of subjects include mammals, e.g., humans, dogs, cows, horses, pigs, sheep, goats, cats, mice, rabbits, rats, and transgenic non-human animals, or birds, e.g., ducks, chicken, geese, and swans. In certain embodiments, the subject is a human. Similarly, the term “host” is intended to include organisms, e.g., mammals, e.g., humans, dogs, cows, horses, pigs, sheep, goats, cats, mice, rabbits, rats, or birds, e.g., ducks, chicken, geese, and swans, and transgenic non-human animals, that harbor a viral strain, nucleotide sequences that recombine via copy-choice recombination, etc.

In one embodiment, the viruses are RNA viruses. In one embodiment, the RNA viruses are single-stranded RNA viruses. In one embodiment, the single-stranded RNA viruses are positive-sense RNA viruses. In another embodiment, the single-stranded RNA viruses are negative-sense RNA viruses. In a related embodiment, the RNA viruses are double-stranded RNA viruses. In one related embodiment, the double-stranded RNA viruses are positive-strand RNA viruses. In another embodiment, the double-stranded RNA viruses are negative-strand RNA viruses.

In another embodiment, the viruses are DNA viruses. In one embodiment, the DNA viruses are single-stranded DNA viruses. In another embodiment, the DNA viruses are double-stranded DNA viruses.

In one embodiment, the viruses are influenza A and/or influenza B viruses. In another embodiment, the viruses are coronavirus viruses, e.g., SARS CoV.

In one embodiment, the protein or nucleic acid sequences are from influenza A and/or influenza B viruses.

In one embodiment, the influenza A and/or influenza B nucleic acid or polypeptide sequences are selected from the group consisting of: HA, NA, NP, PA, PB1, PB2, MP, and NS, or combinations thereof.

In one embodiment, the nucleic acid or polypeptide sequences are obtained by sequencing the isolated viral strains. In another embodiment, the sequences are obtained by sequencing nucleic acid molecules isolated from a subject (e.g., a human or animal) or a tissue sample. In another embodiment, the nucleic acid or polypeptide sequences are obtained from a publicly available database. In certain embodiments, the sufficient number is 5, 10, 20, 30, 40, 50 or more viral sequences.

In one embodiment, the one or more viral genes is at least two, three, four or five or more genes.

In another aspect, the invention provides a method of producing a viral vaccine, comprising: infecting a host animal, host animal cell, cell line, egg cell, bacterial cell, or cell extract which supports viral replication with the parental viral strains identified according to the methods described above; and isolating mutant progeny viral strains from the host animal cell line, egg cell, bacterial cell, or cell extract which supports viral replication.

Viral vaccines of the present invention can be, for example, live vaccines, killed vaccines, attenuated vaccines or subunit vaccines (see, for example, Fields Virology, (1996) Third Edition, Lippencott-Raven Publishers, Philadelphia, pp. 467-469.) Further examples of vaccine production are, for example, Meadors et al. (1986) Vaccine: 179-184, Poland et al. (1990) J. Infect. Disease 878-882, Fenner et al. The Biology of Animal Viruses; New York, Academic Press, 1974:543-586, Saban et al. (1973) J. Biol. Stand. 115-118, and Lowrie et al., DNA Vaccines: Methods and Protocols, Humana Press, New Jersey, 1999.

An attenuated whole organism vaccine uses a non-pathogenic form of the desired virus. Non-pathogenicity may be induced by growing the virus in abnormal conditions. Those mutants that are selected by the abnormal medium are usually limited in their ability to grow in the host and be pathogenic. The advantage of the attenuated vaccine is that the attenuated pathogen simulates an infection without conferring the disease. Since the virus is still living, it provides continual antigenic stimulation giving sufficient time for memory cell production. Also, in the case of viruses where cell-mediated immunity is usually desired, attenuated pathogens are capable of replicating within host cells. Genetic engineering techniques are being used to bypass these disadvantages by removing one or more of the genes that cause virulence.

An inactivated whole organism vaccine uses viruses which are killed and are no longer capable of replicating within the host. The viruses are inactivated by heat or chemical means while assuring that the surface antigens are intact. Inactivated vaccines are generally safe, but are not entirely risk free. Multiple boosters are usually necessary in order to generate continual antigen exposure, as the dead organism is incapable of sustaining itself in the host, and is quickly cleared by the immune system.

One or more polypeptides, or fragments thereof, that are presented by a virus can be formulated into a vaccine that elicits an immune response in a host. These so called “subunit” vaccines often alleviate the safety concerns associates with whole virus vaccines.

In a related embodiment, the method further comprises attenuating the mutant progeny viral strains to make an attenuated viral vaccine. In another embodiment, the method further comprises killing the mutant progeny viral strains to make a killed viral vaccine. In another embodiment, the method further comprises isolating viral antigens, or portions thereof, from the mutant progeny viral strains to make a subunit viral vaccine.

In another embodiment, the invention provides a method of immunizing a subject against a virus comprising: administering to the subject the attenuated virus vaccine in an amount sufficient to immunize the subject. In one embodiment, the subject is a mammal, e.g., a human, in another embodiment the subject is a bird.

In one embodiment, the method of immunizing a subject (e.g., a human or animal) against a virus comprising: administering to the subject a killed, or attenuated, virus vaccine in an amount sufficient to immunize the subject. In another embodiment, the invention provides a method of immunizing a subject against a virus comprising administering to the subject the subunit virus vaccine in an amount sufficient to immunize the subject. In one embodiment, the parental strains are influenza A and/or influenza B viral strains. In another embodiment, the parental strains are coronavirus viral strains.

In one aspect, the invention provides a method of immunizing a subject (e.g., a human or animal) against a virus comprising: administering to the subject a first virus representing the first parental viral strain and a second virus representing a second parental viral strain, the first and second parental viral strains identified according to the methods described herein, in an amount sufficient to immunize the subject.

In one embodiment, the parental viral strains are attenuated prior to administering to the subject. In another embodiment, the parental viral strains are killed prior to administering to the subject. In another embodiment, the method comprises isolating viral antigens, or portions thereof, from the parental viral strains to make a subunit viral vaccine prior to administering to the subject.

In one embodiment, the parental viral strains are influenza A and/or influenza B viral strains. In another embodiment, the parental viral strains are coronavirus viral strains.

In another aspect, the invention provides a viral vaccine composition comprising the parental viral strains identified according to the methods described herein, or antigens, or portions of antigens, therefrom.

In one embodiment, the viral vaccine further comprises mutant progeny viral strains derived from the parental viral strains, or antigens, or portions of antigens, therefrom. In another embodiment, the vaccine comprises two viral strains, or antigens, or portions of antigens from two viral strains.

In another embodiment, the vaccine composition, comprising mutant progeny viral strains, or antigens or portions of antigens therefrom, is made by recombination according to a copy-choice mechanism of two viral strains whose genomes are made up of non-identical nucleic acid sequences. In one embodiment, the two viral strains are parental viral strains identified according to the methods described herein.

In one embodiment, the mutant progeny viral strains are produced by recombination according to a copy-choice mechanism in a host animal. In another embodiment, the mutant progeny viral strains are produced by recombination according to a copy-choice mechanism in cell culture.

In one embodiment, subjects who should be given a viral vaccine can be determined based on the genotype of the current viral strains in a population. Similarly, the type of vaccine a given subject should receive can be determined based on the genotype of the current viral stains in a population. For example, current viral isolated can be classified by the number of polymorphisms that they have. In one embodiment, the polymorphisms are ones that have been identified in isolates from pervious outbreaks. The identification of sequence polymorphisms in a population of viral isolates can be used to form an exposure timeline. This time line can be used to determine the age group susceptibility to a viral infection. For example, a new isolate with a number of polymorphisms identified in 1970 may be less of a concern to those people born prior to 1970, whereas this same isolate may produce more severe infection in those subjects born after 1970. Based on this timeline, medical professionals can determine which subjects should be administered a vaccine, or what vaccine a given subject should receive.

In another aspect, the invention provides a method of identifying the stability of a genome in a population of viruses, comprising: obtaining the nucleic acid or polypeptide sequence of one or more viral genes from a sufficient number of isolated viruses from the population; comparing the number of recombinant viral sequences in the isolated viruses; wherein the greater the number of distinct viral sequences, the greater the instability of the viral genome.

In another aspect, the invention provides a method of identifying the stability of a genome in a population of viruses, comprising: comparing the nucleic acid or polypeptide sequence of one or more viral genes from a sufficient number of isolated viruses from the population; comparing the diversity between parental viral sequences in the isolated viruses; wherein the greater the diversity of distinct viral sequences, the greater the instability of the viral genome.

Genetic stability can be used to measure environmental or experimental effects on genetic stability. This measurement can be determined actively or passively. Thus animals can be immunized and then co-infected with two parental strains and the progeny can be monitored to see the amount of recombination that occurs. This approach can be used to measure the ability of a vaccine to reduce or eliminate recombinants. Similarly, assaying a natural population at different time points can be used to measure environmental effects on recombination. The amount of genetic stability (or instability) can be used to identify times when aggressive intervention is necessary, even in the absence of overt disease.

In another aspect, the invention provides a method of immunizing a subject (e.g., a human or animal) against a virus comprising: administering to the subject mutant progeny viral strains, or antigens or portions of antigens therefrom, made by recombination according to a copy-choice mechanism of two viral strains whose genomes are made up of non-identical nucleic acid sequences.

In another aspect, the invention provides a method of immunizing a subject (e.g., a human or animal) against a virus comprising: determining the parental viral strains in a population of viruses; allowing the parental viral strains to recombine according to a copy-choice mechanism to produce mutant progeny viral strains; administering the parental viral strains, or mutant progeny viral strains, or antigens or portions of antigens therefrom, in an amount sufficient to immunize the subject.

In another aspect, the invention provides a method for identifying parental influenza A and/or influenza B strains in a population of influenza viruses, wherein the population comprises parental influenza A and/or influenza B strains and mutant progeny influenza A and/or influenza B strains, comprising the steps of: obtaining the nucleic acid or polypeptide sequence of one or more influenza A and/or influenza B genes from a number of isolated influenza A and/or influenza B strains from the population, the number sufficient to allow for identification of the influenza A and/or influenza B strains most prevalent in the population, the influenza A and/or influenza B strains having the greatest sequence divergence in the population, or both; identifying the influenza A and/or influenza B strains most prevalent in the population, or influenza A and/or influenza B strains with the greatest sequence divergence in the population, or both;

wherein the most prevalent influenza A and/or influenza B sequences, or the influenza A and/or influenza B sequences with the greatest divergence are the parental influenza A and/or influenza B strains.

In a related embodiment, the invention provides a method of producing an influenza A and/or influenza B vaccine, comprising: infecting a host animal with the parental influenza A and/or influenza B strains identified; and isolating mutant progeny influenza A and/or influenza B strains from the host animal.

In a related embodiment, the invention provides a method of immunizing a subject against an influenza A and/or influenza B virus comprising: administering to the subject a first influenza A and/or influenza B virus representing the first parental influenza A and/or influenza B strain and a second influenza A and/or influenza B virus representing a second parental influenza A and/or influenza B strain, the first and second parental influenza A and/or influenza B strains identified according to the methods described herein, in an amount sufficient to immunize the subject.

In one aspect, the invention provides a method of producing a library of recombinant viral strains comprising: infecting a host cell or animal with two or more viral strains; allowing for recombination of the viruses by a copy choice mechanism of the two or more viral strains, thereby creating a library of viral strains. In one embodiment, the library of recombination viral strains can be isolate for vaccine production.

In a related embodiment, the viral strains may be different species of viruses. For example, the first virus could be influenza A and/or influenza B and the second virus could be a coronavirus, e.g., SARS. In a related embodiment, the identification of a DNA sequence from one species' genome that originated in the genome of a distinct species is indicative that this segment of DNA confers an advantageous property to the virus, i.e., increased infectivity or virulence. Targeting these regions of DNA would provide for effective anti-viral therapy.

In a related embodiment, the library of viral strains can be created in a host cell or animal that has been given an antiviral compound. In a related embodiment, the viral strains that are created in the presence of an antiviral compound are indicative of the antiviral resistant strains that will occur in a population of subjects treated with the antiviral compound.

In another aspect, the invention provides a vaccine composition, comprising mutant progeny influenza A and/or influenza B strains, or antigens or portions of antigens therefrom, made by recombination according to a copy-choice mechanism of two influenza A and/or influenza B strains whose genomes are made up of non-identical nucleic acid sequences.

In other embodiments, art-recognized methods of gene therapy, e.g., RNAi, may be employed to target viral strains, optionally in a strain and/or otherwise sequence-specific manner, e.g., via use of miRNA, siRNA, shRNA, or other such agents.

In another aspect, the invention provides a method for identifying parental coronavirus strains in a population of coronavirus viruses, wherein the population comprises parental coronavirus strains and mutant progeny coronavirus strains, comprising the steps of: obtaining the nucleic acid or polypeptide sequence of one or more coronavirus genes from a number of isolated coronavirus strains from the population, the number sufficient to allow for identification of the coronavirus strains most prevalent in the population, the coronavirus strains having the greatest sequence divergence in the population, or both; identifying the coronavirus strains most prevalent in the population, or coronavirus strains with the greatest sequence divergence in the population, or both; wherein the most prevalent coronavirus sequences, or the coronavirus sequences with the greatest divergence are the parental coronavirus strains.

In a related embodiment, the invention provides a method of producing a coronavirus vaccine, comprising: infecting a host animal with the parental coronavirus strains identified; and isolating mutant progeny coronavirus strains from the host animal.

In another aspect, the invention provides a method of immunizing a subject against an coronavirus virus comprising: administering to the subject a first coronavirus virus representing the first parental coronavirus strain and a second coronavirus virus representing a second parental coronavirus strain, the first and second parental coronavirus strains identified according to the methods described herein in an amount sufficient to immunize the subject.

In one aspect, the invention provides a vaccine composition, comprising mutant progeny coronavirus strains, or antigens or portions of antigens therefrom, made by recombination according to a copy-choice mechanism of two coronavirus strains whose genomes are made up of non-identical nucleic acid sequences.

In another aspect, the invention provides a method of producing mutant progeny viral strains for the manufacture of a viral vaccine comprising; infecting a cell or animal with two non-identical viral strains; allowing for recombination of the non-identical viral strains according to a copy-choice mechanism; thereby producing mutant progeny viral strains. In one embodiment, the method further comprises isolating the mutant progeny viral strains from the host cell or animal.

In one aspect, the invention provides a method of determining the efficacy of a vaccine comprising: obtaining the nucleic acid or polypeptide sequence of one or more viral genes from a number of isolated viral strains from a population that has been treated with a viral vaccine, the number sufficient to allow for number of mutant progeny viral strains in the population; wherein, the lower the number of different mutant progeny viral strain sequences, the greater the efficacy of the vaccine.

In another embodiment, the invention provides a method of predicting the sequence of one or more genes in a mutant progeny viral strain comprising obtaining the sequence of one of more of the genes from a parental viral strain, determining the location of possible recombination events, thereby predicting the sequence of one or more genes in a mutant progeny viral strain. In a related embodiment, the viral strain is selected from the group consisting of an influenza A and/or influenza B viral strain, a corona viral strain, and an HIV viral strain. In another related embodiment, the method further comprises using the predicted sequence of the mutant progeny viral strain to develop a vaccine against said virus.

In another aspect, the invention provides a method of producing mutant progeny viral strains comprising infecting a cell or animal with two non-identical viral strains, allowing for recombination of the non-identical viral strains according to a copy-choice mechanism, thereby producing mutant progeny viral strains. In a related embodiment, the method further comprises isolating said mutant progeny viral strains.

In a related aspect, the invention provides a method of producing mutant progeny virus(es) comprising infecting a cell or animal with two or more non-identical viruses (e.g., ebola and influenza A or influenza B), allowing for recombination of the non-identical viruses according to a copy-choice recombinant mechanism, thereby producing mutant progeny virus(es). In related embodiments, the method further comprises isolating and/or raising vaccine(s) to said mutant virus(es).

In a further aspect, the invention provides a method of producing mutant progeny bacterial strains comprising infecting a cell or animal with two or more non-identical bacterial strains, allowing for recombination of the non-identical bacterial strains according to a copy-choice recombinant mechanism, thereby producing mutant progeny bacterial strains. In a related embodiment, the method further comprises isolating said mutant progeny viral strains. In another embodiment, the method further comprises assessing a phenotypic trait of a mutant progeny bacteria (e.g., drug resistance, assessed, e.g., via compound screening assays).

In a related aspect, copy-choice recombination is responsible for the occurrence of non-mendelian inheritance in certain plants, e.g., Arabidopsis. Thus, the invention provides a method for predicting and/or performing non-mendelian inheritance via copy-choice recombination in plants (e.g., Arabidopsis), provided two or more non-identical parental plants.

In a related aspect, the invention provides a method of predicting a phenotypic trait (e.g., virulence, drug resistance, etc.) of a mutant progeny virus, bacteria or plant through assessment of the range of mutant progeny possible via copy-choice recombination from two or more parental viruses, bacteriae or plants.

In another aspect, the invention provides a method of producing a population of recombinant genes comprising introducing into a cell two or more non-identical copies of a gene, allowing for recombination of the genes, thereby producing a population of recombinant genes. In a related embodiment, the recombination occurs via a copy-choice mechanism. In a related embodiment, the method further comprises isolating one or more members of the population of recombinant genes. In one embodiment, the genes are viral genes. In another embodiment, the genes are from non-viral species, e.g., plants or animals.

In certain aspects, the present invention concerns the genetic transfer of polymorphic sites between strains of influenza. Within the influenza genome, sites of clinical relevance to humans are predicted to be those that enhance the molecular specificity, infectivity, virulence, propagation, etc. of influenza virus within a human subject, as compared to, e.g., an avian subject. An exemplary mutation documented to increase the affinity of the HA protein of H5 strains of virus for human glycoprotein receptors, as compared to avian glycoprotein receptors, is the S227N polymorphism (H3 residue numbering used; by H5 residue numbering, termed the S223N polymorphism) featured in certain embodiments of the present invention (Hoffmann et al., Proc. Natl. Acad. Sci. 102: 12915-20). Additional polymorphisms within the influenza genome predicted to impart, e.g., molecular specificity, infectivity, virulence, propagation, transmission, etc., of heightened human impact to the influenza virus include documented polymorphisms in HA (e.g., mutations at residue(s) 190, 225, 226, 227 (e.g., S227N) and/or 228 (G228S) (H3 residue numbering) and/or residue(s) 36, 83, 86, 120, 155, 156, 189, 212, 263 (H5 residue numbering)), PB2 (e.g., mutations at residue(s) 627 (e.g., E627K, shown to be important to mammalian adaptation of the 1918 pandemic influenza virus), 199 (e.g., A199S), 475 (e.g., L475M), 567, 627 and/or 702), PB1 (e.g., mutation(s) at residue(s) 54 (e.g., K54R), 375, 383, 473, 576, 645 and/or 654), and PA (e.g., mutations at residue(s) 241 (e.g., C241Y), 312 (e.g., K312R), 322 (e.g., 1322N), 55 (e.g., D55N), 100 (e.g., V100A), 312, 322, 382 (e.g., E382D) and/or 552 (e.g., T552S)) (Hoffmann et al., Proc. Natl Acad. Sci. 102: 12915-20; Taubenberger et al., Nature 437: 889-93; Gambaryan et al., Virology 344: 432-38; Stevens et al., J. Mol. Biol. 355: 1143-55). Additional exemplary mutations identified in the influenza genome and of potential human impact are presented in Table 21.

Both known and newly-identified mutations in the influenza genome can be tested for their potential impact on human molecular specificity, infectivity, virulence, propagation, transmission, etc., via art-recognized methods (e.g., propagation of influenza in, e.g., Vero or MDCK cells, as compared to chicken embryo cells; molecular modeling approaches to identify potential impact of mutations upon, e.g., HA binding to receptors and/or impact of mutants upon function of the heterotrimeric polymerase complex (PA, PB1, PB2)). In certain embodiments of the present invention, the molecular affinity of the HA protein of influenza for specific receptor glycoproteins (e.g., glycoproteins more prevalent in the human respiratory tract, such as α-2-6-linked sialic acids, as compared to glycoproteins more common in the avian enteric tract) is directly assayed in vitro via use of glycan microarrays. Glycan microarrays as described in Stevens et al. (J. Mol. Biol. 355: 1143-55) allow for rapid assessment of the impact of any mutation in the HA protein of influenza upon the affinity of HA for an extensive panel of glycan modifications, a selection of which are more prevalent in the mammalian respiratory tract. Within the present invention, assay of mutant HA proteins for glycan specificity can be performed on either parental strains of virus (e.g., to prioritize geographic tracking of specific mutation(s) of heightened predicted, e.g., human/clinical impact, on the basis of an observed glycan microarray binding profile) or progeny strains of virus (e.g., to perform an in vitro assessment of specific progeny strains of virus predicted to arise from two or more parental strains of virus). Details regarding performance of such assays can be found in Stevens et al., the contents of which are incorporated herein by reference in their entirety.

Certain aspects of the present invention involve the mixing of two or more parental strains of virus for purpose of ascertaining the identity of progeny strains of virus arising therefrom. While such mixing experiments can be modeled by hand and/or in silico, physical mixing of parental strains can be performed either in vitro or in vivo by art-recognized approaches of propagating influenza virus. For example, in vitro mixing of parental strains of influenza virus can be performed in a wide range of cell types, including chicken embryonic cells, and a number of mammalian cell lines, e.g., Vero (derived from African green monkey kidney) and MDCK (canine kidney) cells (refer to Mochalova et al., Virology 313: 473-80). In certain embodiments of the present invention, performance of such parental strain mixing experiments in mammalian cell lines, particularly primate cell lines, is preferred, for purpose of selecting in favor of viral strains more likely to impact human specificity, propagation, virulence, infectivity, etc. (in certain instances, propagation of influenza in e.g., chicken embryo cells, might be anticipated to select away from human/primate-specific strains of virus, potentially limiting the information to be gained via performance of mixing experiments in such cells). Accordingly, the viral strain mixing experiments of the present invention may be performed in any art-recognized cell line capable of propagating the influenza virus (refer to “Influenza Vaccine Production” section below).

In certain embodiments of the present invention, mixing of parental viral strains can also be performed in vivo. For example, avian and/or mammalian host organisms can be infected with parental strains of virus (including attenuated strains of virus) in order to discern the identity of specific progeny strains of virus arising from such combined infection of host organisms with the parental strains. Host organisms can include any avian and/or mammalian organism, including, e.g., mammals, e.g., primates, dogs, cows, horses, swine, sheep, goats, cats, mice, rabbits, rats, and transgenic non-human animals, or birds, e.g., ducks, chicken, geese, turkeys, quail and swans.

Two parental viral sequences can be combined in vitro, in vivo, or in silico, with the rules of the present invention allowing for enhanced prediction of which mutant progeny virus(es) will exhibit a monitored trait. The present invention can therefore be applied, e.g., to drug screening approaches, vaccine production, diagnostic (kit) production, etc.

It is understood that zoonotic dieoffs (e.g., ducks, swans, quail, swine), especially in particular geographic areas, can be used to predict parental strains that will contribute to progeny strains of virus via the gene transfer events of certain aspects of the present invention.

It is understood that the invention also encompasses the application of predicting the emergence of influenza strains from sequences derived from domestic and/or farm animals (e.g., swine isolate sequences). As set forth in the Examples below, such animals, e.g., swine, may act as a sequence reservoir over longer spans of time than are normally seen in the evolution of rapidly spreading migratory bird and human strains of influenza. Such sequence reservoirs may then be drawn upon via recombination with, e.g., migratory bird and/or human sequences, contributing as parental strains to future progeny strains of influenza.

As also set forth in the Examples below, mapping of parental strains through use of appropriate probe sequences to individual influenza haplotypes can reveal the transition of a sequence from, e.g., an H1 strain to a more aggressively virulent H5 strain. Observation of such strain-transitional flow of influenza sequence can reveal polymorphic sequences of particular importance for vaccine development against future progeny strains of influenza.

### Influenza Vaccine Production

Certain embodiments of the invention involve production of vaccines to, e.g., progeny viral strain sequences of the invention. The generation of such vaccines can be performed by any art-recognized method. Exemplary methods of vaccine production involve production/propagation of virus, purification and formulation of virus and/or viral components for use as vaccines, and administration of such vaccines.

Viral production systems known in the art include, e.g., those described in U.S. Pat. Nos. 6,544,785; 6,649,372 (featuring methods for generating in cultured cells (e.g., Vero cells) infectious viral particles of a segmented negative-strand virus without using helper virus, including vaccines and compositions produced by such methods); 6,146,642 (featuring a recombinant RNA molecule comprising a binding site specific for an RNA-directed RNA pol of a Newcastle disease virus (NDV)), linked to a viral RNA containing a heterologous RNA sequence; 6,669,943 (featuring an attenuated influenza virus with modified NS1 gene and interferon antagonist phenotype, including vaccines and pharmaceutical formulations made therefrom); 6,573,079 (featuring methods of vaccine production via propagation of an attenuated influenza virus having a mutation in the NS1 gene that reduces the cellular interferon response); 5,989,805 (featuring methods for propagating and/or preparing an avian virus, e.g., influenza, using chicken embryonic cells); 5,948,410 (featuring influenza surface antigen vaccines from influenza virus propagated in animal cells (e.g., canine kidney cells (MDCK)) and substantially free of host cell DNA); 4,552,758 (featuring a method of preventing influenza A virus in humans; 4,552,757 (featuring an influenza vaccine for use in non-avian animals, the vaccine comprising NP or M proteins from specified strains); 6,344,354 (featuring a vaccine comprising a replicated mammalian influenza virus passaged in cells that are not eggs, including methods of vaccination using the same); 5,824,536 (featuring methods of making vaccine via infection of mammalian cells (e.g., Vero cells) and culturing in trypsin-containing media); 6,048,535; 6,406,702 (featuring multivalent poultry vaccines safe for ovo inoculation comprising the agents Newcastle disease virus (NDV) and, e.g., influenza virus, also featuring vaccine methods using such vaccines); 6,322,967 (featuring a method of making influenza virus with a modified PB2, an influenza virus made according to such a method, and a method of treating humans with such a vaccine); 6,146,873 (featuring methods for producing orthomyxovirus (influenza) virus using monkey kidney cells in protein-free media); 5,753,489 (featuring the methods of 6,146,873, wherein cells are instead adapted to serum-free media); 4,500,513 (featuring methods of preparing influenza vaccine using cell culture and a proteolytic enzyme (e.g., trypsin)); 5,756,341 (featuring methods of producing influenza vaccine antigens in serum-free cell culture using HA with a modified cleavage site); and 5,698,433 (featuring methods of preparing influenza virus using avian embryo cells and a serine protease). The preceding U.S. patents are incorporated in their entirety herein by reference.

Vaccine purification and formulation methods and compositions described in the art include, e.g., U.S. Pat. Nos. 6,060,068 (featuring a vaccine (e.g., for equine influenza) that comprises IL-2 as a coadjuvant); 6,451,325 (featuring an influenza virus vaccine formulation comprising metabolizable oil adjuvant); 5,709,879 (featuring an influenza virus vaccine formulation comprising metabolizable oil adjuvant in a liposome possessing net negative charge); 6,743,900 (featuring methods of preparing an influenza vaccine formulation using a proteosome preparation); 6,387,373 (featuring an influenza vaccine formulation comprising an oil-containing lipid adjuvant); 5,795,582 (featuring an influenza vaccine formulation comprising a dendrimer adjuvant); 5,919,480 (featuring an influenza vaccine formulated as a liposome comprising a cytokine, including methods of administration of same); 5,639,461 (featuring an influenza vaccine 99% inactivated by heat and formulated with thimerosal, including methods of administration of same); 3,919,044 (featuring methods of purifying and concentrating virus (e.g., influenza) using filtering and cationic exchange); 4,000,257 (featuring methods of extracting pyrogens and endotoxins from an influenza virus vaccine); 6,231,860 (featuring stabilizing agents (e.g., urea) for attenuated viral vaccines (e.g., influenza vaccine)); and 6,048,537 (featuring methods of preparing purified mixtures of influenza viral antigens by fragmenting live virus).

Methods of use and/or administration of anti-viral vaccines known in the art include, e.g., U.S. Pat. Nos. 5,916,879 (featuring a method of immunizing an avian with DNA encoding influenza H5); 5,643,578 (featuring methods of immunizing a vertebrate with DNA encoding HA of an infectious agent (e.g., influenza)); 6,159,472 (featuring a method of immunizing an avian intradermally with a vaccine comprising inactivated immunogen (though the vaccine can comprise, e.g., a live influenza immunogen)); 6,682,754 (featuring a method of inducing immunity via an implant comprising an immunogen (e.g., derived from influenza virus)); 5,817,320; 5,750,101 (featuring a method of ovo-immunization via administration of a vaccine into an egg air cell); 6,506,385 (featuring method of immunizing against avian viral disease via administration of live virus and interferon to an egg); and 5,149,531 (featuring a method of treating a subject (e.g., a bird subject) with a cold-adapted live influenza vaccine).

### EXEMPLIFICATION

Throughout the examples, the following materials and methods were used unless otherwise stated.

### Materials and Methods

In general, the practice of the present invention employs, unless otherwise indicated, conventional techniques of chemistry, molecular biology, recombinant DNA technology, immunology (especially, e.g., antibody technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley & Sons (1992).

### Example 1

### Influenza a Emergence and Evolution Via Recombination

Influenza A is thought to evolve gradually via point mutations and abruptly via reshuffling of its eight segmented genes. Here, Influenza A evolution has been shown to be driven by recombination in hosts infected with two distinct viruses. Most polymorphisms of closely related viruses are bimorphisms, involving third base codon changes, which are silent at the protein level. The recombination generates both versions of the nascent genes and both viruses are viable. The recombination redistributes existing polymorphisms, allowing prediction of the genetic composition of new viruses, before they emerge. This recombination mechanism is common. It generates pandemic H5N1 influenza, as well as most or all, rapidly evolving genomes.

The looming H5N1 flu pandemic has attracted considerable attention (Peiris et al 2004; Fouchier et al 2005; Osterholm 2005). To determine the molecular mechanisms behind the evolution and emergence of H5N1, recent sequences of isolates from Hong Kong were compared to determine the genetic basis for the evolution of a pandemic strain that has spread through eastern Asia and caused human fatalities in Vietnam, Thailand, and Cambodia.

Influenza has a segmented genome and the reassortment of the eight genes has been used to classify the H5N1 isolates (Guan et al 2002, Alexandr et al 2003). Changes in influenza genetic composition have been described as drifts and shifts (Webster et al 1992). The drifts have been characterized as gradual changes due to replication errors by an RNA polymerase lacking a proof-reading function. Shifts are thought to involve more dramatic changes in genetic composition due to reassortment of the eight sub-genomic RNAs.

Analysis of the changes of H5N1 isolates indicated that both drifts and shifts were caused by homologous recombination, which generated complementary genomes. Dual infections lead to the recombinants, which resemble point mutations when two closely related viruses recombine. The same mechanism between more distantly related virus can produce much more dramatic changes and create two viable genomes which are complementary. Most of the polymorphisms are transitions, so the positions are largely binary and most of the RNA changes are synonymous. Thus, the polymorphisms in the genetic population are largely bimorphisms represented by either a purine or pyrimidine. The redundancy in the genetic code produces synonymous changes for almost all third base transitions.

Although many changes are found throughout the database indicating a large number of alterations would produce viable progeny, the changes are limited largely by the resident gene pool and the composition of the recombinants is related to the prevalence of the polymorphisms in co-circulating genotypes. The newly formed recombinants can be predicted by the prevailing gene pools.

These complementary polymorphisms are used in virtually all biological systems. They are common in rapidly evolving viruses, but similar mechanisms drive repair of polymorphisms at the cellular level. Moreover, non-homologous recombination is used to link together viral genomes that have similar pathologies such as Ebola and pandemic H1N1 and H5N1 influenza.

Evolution of pandemic H5N1 can be traced to 2001 H5N1Hong Kong isolates. The live market isolates formed five groupings based on reasserted genes (Guan et al 2002). Representative isolates generated a neurotropic version isolated from mouse brain (Alexander et al 2003). The isolates in Hong Kong had major polymorphisms that were present in at least 20% of the isolates.

The polymorphisms for all eight genes were examined. For each gene, the isolates segregated out into two major genotypes designated Allele 1 and Allele 2. Allele 1 was composed of Group A and for some genes, Group B. Allele 2 was generally Groups C-E. These groupings were present across all eight genes and the polymorphisms were coded with regard to the emerging pandemic strain found in Vietnam and Thailand. The two alleles complement each other and for most positions the polymorphisms were bimorphisms incorporating a purine or pyrimidine at third base positions, thereby producing synonymous changes.

The complementary nature of the bimorphisms suggested the two alleles were generated via homologous recombination. The use of only a purine or pyrimidine at a third base position generates two RNA versions of the same protein. The number of bimorphisms for each gene suggested some of these genes had already recombined to generate one version that was highly homologous to the pandemic strain and another version that contained the alternate purine or pyrimidine. Thus, for PB2, HA, NA, and NS the number of polymorphisms was small (11-39) and the bimorphisms that matched the pandemic strain were evenly divided between two alleles. In contrast, PB1 had already recombined to place most of the matching bimorphims in allele 1 and there were 111 polymorphisms. Similarly, PA had also recombined so most of the matching bimorphims were in allele 2 and there were 114 polymorphisms. M is a smaller gene and not as genetically diverse, so there were only 29 polymorphisms and most that matched the pandemic gene were in Allele 2. NP was somewhat unusual. There were 63 polymorphisms that were evenly divided, but there were 61 additional polymorphims that were not in either allele, but were already in Group E which was defined by an NP gene that was novel to the other isolates in Hong Kong.

The genotype data obtained also revealed more limited recombination, which could be seen in the paired isolates. In PA, the chicken isolates YU822.2 were from Group A and matched Allele 1. However, the two sequences diverged between positions 933 and 1143. There were 18 bimorphisms in this region and only two positions were the same in both isolates. For the 16 positions that diverged, the mouse brain isolate matched allele 2 at all 16 positions. A similar crossing over event was seen in the PB1 gene. NT873.3 was in Group E and matched allele 2. However, the two sequences diverged between positions 1374 and 1509. The were 10 bimorphisms and NT873.3 matched the consensus sequence for allele 1 at all 10 positions, identifying another cross-over event that had caused a short region of allele 1 to be detected in allele 2.

The result of much more extensive recombination was also seen in the Hong Kong isolates from 2002 and 2003 (Strum-Ramirez et al 2004). The same bimorphisms were observed as previously. However, the more recent isolates have recombined the polymorphisms from allele 1 and 2 to create a new allele that is highly homologous to the pandemic strain. This recombination was present in all 8 genes. The bimorphisms that were present in the pandemic strain but were missing from the Hong Kong isolates from 2001 were also observed in this analysis. The genomes recombined in the bimorphisms in the first consideration also had most of the polymorphisms not present in Hong Kong in 2001. Thus, not only were the bimorphisms on allele 1 and 2 combined, but the genes now contained most of the missing bimorphisms, which were likely acquired outside of Hong Kong. Bimorphisms not present in the 2002 and 2003 Hong Kong isolates were also considered. These bimorphisms can be found in more distantly related mammalian isolates. However, the bimorphisms from the initial analysis were shown to have recombined and added most of the bimorphisms that were not present in Hong Kong in 2001.

Additional examples of recombination were also observed. Polymorphisms for the four genes for the replication complex were compiled. These bimorphisms were defined by two isolates from the same chicken in Hong Kong, 31.2 and 31.4. The large number of polymorphisms observed was due to the complementary relationship between 31.2 and 31.4. 31.2 has recombined and is closely related to the pandemic strain. In contrast, the corresponding opposite purine or pyrimidine is found in the 31.4 sequence. These two complementary sequences act as parents of additional recombinants found in Hong Kong live markets, as displayed in the first four panels.

The number of bimorphisms was high for each of the four genes and the two parental genes were homologous or non-homologous to the pandemic genes. For PB1 there were 181 bimorphisms. The recombinant 37.4 was virtually identical to 31.2 through position 1418. The remainder of the available sequence (through position 1890) matched the pandemic strain. Included in panel A is an H5N1 isolate from Fujian province, which is homologous to the 31.2 sequence. The Fujian sequence is complete and was used to define the polymorphisms in the position of the sequence absent for 31.2. The Fujian sequence is one of many sequences outside of Hong Kong that is homologous to 31.2.

For PB2, only a partial sequence was available, but in the 3′ half of the gene beginning at position 1023, there were 144 bimorphisms. For this gene, YU100 is the recombinant and it was virtually identical to the pandemic gene through position 1665 and then matched 31.4 for the remainder of the gene. For comparison, homologous genes from other serotypes (H7N7, H7N3, H9N1, H9N2) from around the world were aligned, and demonstrated that the sequences with the alternate purine or pyrimidine were widespread and not limited to a novel gene found in Hong Kong.

For PA, there were 191 bimorphisms and both YU100 and 37.4 were recombinants on this gene. YU100 matched 31.4 through position 1449 and then switched to the 31.2 sequence. In contrast, YU100 matched 31.2 through position 873 and then switched to the 31.4 sequence.

For NP, the relationship between the two Hong Kong parental strains had switched. 31.2 was highly homologous to the pandemic strain, to which 31.4 was distantly related. 37.4 was a recombinant in NP, sharing bimorphisms with 31.2 through position 789 and then matching 31.4 for the 3′ half of the gene.

Thus, the two parental sequences found in chicken 31 were observed, as well as one or two recombinants that have a single crossover point. The two parental sequences in chicken 31 are common. The pandemic version was found in genotype Z isolates (Guan et al, 2004) throughout Asia and the sequences with the opposite purine or pyrimidine were found in H5N1 isolates throughout Asia, as well as other serotypes throughout the world. Thus, recombination produced two genes, which differed significantly at the nucleotide level, but were highly homologous at the protein level.

Recombination was not limited to the internal genes. Evidence of recombination in NA was also observed, using sequences from H9N2 isolates in Korea. In the 5′ half of the gene, two swine (S452 and S81) shared sequences with a chicken sequence (S1), while the remaining 3 swine sequences (S83, S109, S190) formed the opposite bimorphic sequence. However, at position 660, two of these swine sequences switched to the alternate sequence.

A Korean avian isolate (S16) was a recombinant in PA. The sequence for the first 231 bp was virtually identical to two H9N2 isolates from Hong Kong/Guangzhou. There was only one bp difference with a 2003 avian isolate, and only 2 bp differences with a human H9N2 isolate from 1999. These homologies suggested dual infections between the Korean avian isolate and isolates from Hong Kong. This association was increased by the sequence of the M gene in S16. It was an exact match with a H9N2 1998 swine sequence (10) or differed by a single bp with a second 1998 swine isolate (9).

Recombination could also be found in human flu genes. Two Korean swine isolates displayed PB2 recombinants between the human lab strain WSN/33 and the 2004 Korean swine isolates. A match at the 5′ end of the Genbank sequence of S109 with WSN/33 was observed. This match extended to the 5′ end of the gene and a similar match was found between WSN/33 and S81.

Human/human recombination was also demonstrated. The 5′ half of HA in 2002 Korean isolates matched current H3N2 isolates, represented by the Wyoming vaccine sequence. These Korean isolates matched Korean isolates from 1991 at the 3′ portion of the gene. Sequences closely related to the 1991 Seoul sequences were identified worldwide in the late 1980's, but were absent from HA sequences at Genbank from 1991 to 2002, only to re-emerge as recombinants.

### Recombinant Evolution of Influenza

Influenza evolution has been described as a series of drifts and shifts. Drifting was thought to be driven by point mutations generated by an error prone polymerase, while shifting was linked to the reassortment of the 8 sub-genomic influenza RNAs. However, the present invention shows drifts and shifts occur by recombination, and has provided a mechanism for the genetic diversity seen in viruses and other gene systems, and in particular influenza.

Reassortment between influenza subtype H5N1 and other subtypes has been observed previously. The H5N1 1997 isolates from patients in Hong Kong were reassortants (Guan et al 1999). Internal genes were closely related to genes found in H9N2 and H6N1 isolates. However, this constellation of genes was not seen after H5N1 culling in Hong Kong in 1997. H5N1 was again isolated from humans in Hong Kong in 2003 and these isolates had a very different constellation that was called the Z genotype (Guan et al 2004; Chen et al 2004). Later that year, a related constellation was designated Z+ and this group was found throughout Asia by the beginning of 2004. However, these isolates had regional specific polymorphisms and isolates from patients in Vietnam and Thailand contained polymorphisms unique to those two countries or uniquely shared between those two countries. These polymorphisms will be described elsewhere, but in general the polymorphisms were not found in other H5N1 isolates, but serotypes commonly found in mammals. The biological differences between the Z+ genotypes were due to the polymorphisms, not reassortment. Moreover, the genetic sequences adjacent to these polymorphisms can be used to define serotypes and temporal changes. Similarly, the differences between isolates from mouse brains and the parent virus were due to polymorphism, and not reassortment. Some of the differences were clearly generated by short stretches of recombination, while others resembled point mutations. The acquisition of a mammalian polymorphism linked to virulence in humans (G1906A leading to E627K in the PB2 gene) has led to increased virulence (Hatta et al; Fouchier et al) and has been associated with neurotropism (Lipatove et al; Govorka et al; Thanawongnuwech et al; Mase et al; Chen et al, Lui et al). The homologous region exploited can be in another location within the gene or in another influenza gene. The present findings have identified that such homologous regions, also referred to as “homology islands” herein, are an important source of genetic variability for the emergence of new viral strains and substrains, thereby contributing to a genetic transfer event(s). Compilation of e.g., relational databases comprising homology island sequence(s) for one or more viruses (and/or cells) can therefore allow prediction/anticipation of the characteristics of emerging and future strains of virus, especially where such sequence information and/or distinct homology islands are correlated with clinical and/or pathological characteristics/outcomes.

Although other polymorphisms looked like point mutations, the polymorphisms were not due to recent mutations. They could be found in mammalian serotypes. Similarly, many of the polymorphisms found in the H5N1 pandemic strain could be found in previously-circulating H5N1 isolates. However, as shown herein, these polymorphisms merged via recombination, frequently involving co-circulating haplotypes. These polymorphisms were largely bimorphisms, which were merged via recombination. This process created two viruses simultaneously, which differed in third base codon positions. Since most of these differences were transitions, most of the protein changes were synonymous because transitions in the third base position of 60 of the 64 codons create a synonymous change.

In Hong Kong in 2001 there were two major closely related genotypes circulating. In genes that had not recently recombined, the new recombined bimorphisms looked like point mutations. Paired samples demonstrated short segments of recombination. Some of the genes had already recombined which led to one haplotype with a large number of bimorphisms that matched the pandemic strain and an opposite version that had a large number of bimorphisms that did not match. Further recombination between these two dissimilar haplotypes created new genes that were chimeras with one end of the gene from one parent and the other half from another.

In one mechanism, a larger number of crossover events happen in a dual infection in one host; in another mechanism, the multiple crossover events accumulate via a series of dual infections. Some of the recombinants were generated by a single cross over near the center of the gene, while others involved a short region of a few hundred bp, or even much shorter regions. Homolgy searches demonstrate strain and temporal specifity, for regions as small as 7 bp. The number of reassortments and recombinations identified in subsequent isolates is much less than the theoretical number that could be generated via a dual infection, showing that some selection was involved to allow the new genetic combinations to become fixed.

Recombination is not limited to H5N1 or avian genes. Human genes evolve using the same mechanism and in the Korean swine sequences, genes that were half human and half avian were found. In the example given above, the recombination was with viruses that had been widespread in the late 80's and early 90's, but had disappeared from sequences at GenBank for 10 years. Thus, the sequences can be quite stable and reappear within the populations at a later date. These data show that in the absence of recombination, the fidelity of replication is exceedingly high and the conserved sequence can reappear. This reappearance is occurs via acquisition of avian sequences present in non-human sources, which can evolve more slowly in the absence of recombination. This conservation of sequence identity can also be found in highly evolving environments. In a 2003 Korean isolate (Choi et al.), there was evidence of dual infections with isolates from Hong Kong, a region that has produced rapid genetic change. However, exact copies of the M gene from a 1998 swine isolate in Hong Kong could be found in a Korean avian isolate five years later. These data indicated that the polymerase is not error-prone, or errors are corrected. The diversity seen every year in avian or human isolates is not due to recent mutations. Instead, the changes are clustered bimorphisms, which are distributed via recombination and the distribution can involve avian genes acquiring mammalian polymorphisms or mammalian genes acquiring avian polymorphisms.

The evolution of virus via recombination was stable. The recombinations seen in 2001 strains in Hong Kong were still present in the 2004 pandemic strains. Hong Kong isolates provided a window on the recombination process, but the same types of recombination were seen elsewhere, including isolates that had recombined genes at an earlier date. Comparison of human H1N1 flu isolated in the 1930's, such as WSN/33, with classical swine H1N1 also isolated in the 1930's, identified complementary bimorphisms found in the 1918 pandemic strain. Recombination has been used to define the emergence of the 1918 pandemic strain (Gibbs et al) although the interpretation of the data has been questioned (Worobey, et al). Recombination has been noted as an occasional driver of rapid evolution (Lai, 1992; Woroby & Holmes, 1999), but the mechanism was believed to play a larger role in positive sense RNA viruses (Chare et al, 2003)

These data have several practical applications, especially with their description of high-frequency recombination. Since the newly-formed recombinants were generated by crossovers between the parental genes, new recombinants could be predicted based on the prevalent genomes in the area. The recombinants align between the two dominant genotypes and complementary versions of the recombinants can be generated. The bimorphisms can be used to determine the origins of the recombinants and can also be used to predict the sequence of the complementary version, since the changes are largely transitions. Therefore, one version predicts the sequence of the complementary version. These rules can be used to identify both future and past haplotypes, which have applications in vaccine development. The rules are also useful for identification of short regions of homology, which may not be isogenic

The applications of the rules described herein extend well beyond influenza evolution. Viral sequences at GenBank display high rates of transitions at the codon third base position. The other viruses also use bimorphisms for rapid evolution and complementary versions of viruses are abundant. These complementary relationships can be seen for coronaviruses including SARS (Mara et al 2003; Rota et al 2003), NL63 (va der Hoek et al 2004) and HKU-1 (Woo et al 2005). These relationships will be described elsewhere.

These relationships can also be found in other systems and can explain the gene repair of higher organisms such as that recently described for Arabidopsis HTH genes. Eleven polymorphisms for the Arabidopsis HTH genes were characterized (Lolle et al 2004), and all eleven were transitions. However, these transitions were selected from mutagenized stocks, so they were not third base transitions and the changes were non-synonymous (Krolikowski et al 2003). However, like influenza, the changes were template-driven and specific. They were found at higher frequencies and under various conditions (Lolle et al 2004). Thus recombination in double-stranded RNA versions of genes can explain the non-mendelian inheritance observed after self fertilization in F3 progeny of Arabidopsis.

The high frequency recombination in the viral genomes is driven by dual infections. However, dual infections can also play a role in more dramatic evolution involving unrelated genomes. An 18 bp region of H5N1 HA can be found in the Ebola spike gene (HLN in preparation). This particular region contains regional specific bimorphisms in both genomes. Thus, the isolates from Vietnam and Thailand have a specific bimorphism that is not found in earlier H5N1 isolates. Moreover, a second polymorphism generates the HA sequence found in the 1918 pandemic HA. Thus, the high frequency of homologous recombination seen in hosts infected by the same class of virus also extends to viruses of different classes leading to sharing of sequences which can be linked to similar clinical manifestation such as excessive hemorrhaging seen in humans or animals infected with Ebola, H5N1, or H1N1. Details of these relationships will be presented elsewhere.

Recombination is a strong driver of rapid evolution. In influenza, recombination can produce both drifts and shifts and the same mechanisms have been adopted universally for rapid evolutionary change.

### Example 2

### Copy-Choice Recombination Between Distinct Viruses

The signature of copy-choice recombination can be observed in the mutant progeny of distinct types of parental viruses. A tract of 18 nucleotides in length was observed to have been conserved between Ebola in Africa and the 1918 flu pandemic, connected through intermediate mutant progeny strains of H5 influenza strains. Copy-choice recombination, when combined with selective pressure, can therefore act to conserve blocks of sequence between distinct parental viruses. The conserved block of 18 nucleotides observed likely encodes a small RNA, e.g., a miRNA, possessing functional activity. Similar recombination of sequences from distinct viruses has been observed for SARS, IBV and astroviruses (where a conserved 3′ stem loop structure is shared); foot and mouth disease and Newcastle diseases; and HIV and coronavirus.

### Example 3

### Copy-Choice Recombination Between Influenza A and Influenza B

Recombination is the molecular mechanism for rapid evolution and is used frequently by rapidly evolving influenza viruses. Influenza A is the virus that receives the most attention from the medical community because it causes periodic pandemics. Influenza B is generally considered a milder version of the virus. Since recombination recycles prior polymorphisms, sequence searches can identify parental strains and characterize individual polymorphisms. Use of small stretches of nucleotides can be used to characterize these polymorphisms for the development of vaccines for emerging viruses and for the tailoring of vaccines to individual age groups or regions experiencing localized outbreaks.

These probes have produced profiles that address critical areas related to public health. The current pandemic of H5N1 in Asia has been used herein to illustrate the application of this technology. One alarming aspect of H5N1 infections has been the observation of die-offs of large number of migratory waterfowl infected with H5N1 at the Qinghai Lake Nature Reserve. Although H5N1 can be lethal to humans, the same strain can replicate in the guts of waterfowl without obvious ill effects. Although the sequences of the H5N1 isolated from Qinghai Lake are not yet in the public domain, recent publications show that the isolates were reassortants with 3 genes (HA, NA, NP) closely related to a chicken isolate from Shantou Province while the other 5 genes (PB2, PB1, PA, MA, NS) were closely related to a peregrine falcon isolate from Hong Kong.

Probes representing the polymorphisms in these two isolates were used to characterize the polymorphisms in these two representative isolates and the polymorphisms were found in a wide spectrum of sero-types normally found in migratory birds, showing that the genetic flow of the polymorphisms was from the wild birds to the domestic poultry, and not vice versa. This information was important for identifying parental sources of novel polymorphism that can emerge in new isolates and highlight the seasonality of these events. These findings also revealed the importance of new lethal versions of H5N1 which can now be transmitted throughout Asia.

Probing with short sequences to search the flu database produced an output of aligned sequences, with the output grouping strains that contained the same polymorphism. These data provided evidence of transmission of polymorphisms between Influenza A and Influenza B strains by copy-choice recombination.

The monitoring of polymorphisms also indicated that H5N1 was expanding its host range to mammals by acquiring polymorphisms normally found in humans. Human polymorphisms have been linked to H1, H2, and H3 in influenza A as well as the HA gene in influenza B. Most of the reported human cases of H5N1 have been in Vietnam and Thailand and isolates from those countries have polymorphisms not commonly found in H5N1 isolates. Probing the influenza database with short sequences representing these regions showed that the sequences were found in mammalian Influenza A isolates having H1, H2, H3 as well as HA from Influenza B. These observations also applied to NA in influenza A and B. Although H5N1 already had N1, the molecular probing identified polymorphisms normally found in N1 in H1N1 isolates.

These results showed that recombination between avian and mammalian isolates has already occurred and the 2005 sequences showed that such changes continue to occur, even though public health officials are focused on reassortment and random mutations, instead of recombination, as driving the evolutionary change.

In addition, some of these polymorphisms represented polymorphisms that were present at earlier times and had largely disappeared from the human population. Reintroduction of the polymorphisms can have age specific effects, targeting younger people who have not been previously exposed to such polymorphisms. These polymorphisms can be used to make age specific vaccines.

Monitoring of the polymorphisms showed that some were found in other viruses such as the prior example of identical 19 nucleotide sequences in H5 and Ebola spike gene or closely related pandemic H1. Thus, these polymorphisms can provide important functions and can also be used to modify other genes. The modifications were also important in viral assembly because small regions were used in multiple genes.

Thus, the polymorphisms can also occur in different regions of the same gene or different genes, reflecting changes that happen over longer terms. But such changes can have near term consequences. These changes were less frequent than the homologous isogenic recombinations, but can act as donor sequences.

Thus, recombination can occur in the same location of the same gene, a different location of the same gene, or in an unrelated gene. These regions can also be found in unrelated viruses, such as in the Ebola, influenza A example (Example 2).

Table 1 traces the polymorphism A287C in the influenza HA gene. A probe that used the 18 nucleotide upstream sequence was specific for recent waterfowl isolates at Qinghai Lake, as well as the most closely related isolate from a chicken in Shantou in Gunagdong province. A shorter probe that used the 13 nucleotides upstream from the polymorphism traced the polymorphism through the Los Alamos database. All exact matches were in the HA gene and all serotypes were H5. The probe first matched was in Asia in 1975 and matched H5N2 and H5N3 serotypes in the 70's For strains from the 80's, the probe matched Ireland and Potsdam in serotypes H5N8, H5N6, and H5N2. After a 5-year hiatus, this sequence appeared in a turkey in England in the first H5N1 serotype. After a six year hiatus it appeared simultaneously in southeast Asia and Europe. In Asia, the H5N1 serotype appeared in a patient who was among the 18 people identified with H5N1 infections in Hong Kong. The sequence was also in H5N3 ducks in Singapore. In Italy, it was in domestic terrestrial birds in serotypes H5N1 and H5N9. After another 5-year hiatus, the sequence appeared in Europe and Asia in 2003. It was in a new serotype in the Netherlands, H5N7, as well as the H5N1 Shantou chicken and an H5N3 duck. In 2005, the sequence appeared in waterfowl at Qinghai Lake.

The polymorphism was also present in isolates that had a C to T transversion 7 nucleotides upstream of the polymorphism. This probe also identified H5 isolates and all but three were H5N2. The other three were H5N1, H5N2, and H5N9. Almost all of these isolates however, were in North America. The first isolate was in 1973 from a turkey in England. In strains of the 80's, the sequence was in waterfowl in the United States. In 1993, the sequence was detected in an emu. Most of the isolates from 1993 to 1998 were from birds in Mexico. It appeared in a mallard in 1999 in Europe and in primorie in Russia in 2001. In 2003, the sequence was in the H5N2 isolate from the outbreak in Taiwan.

TABLE 1
Probing polymorphism A287C of the HA Gene
A287C
CCAATGTGTGACGAATTCc

| | | | |
|---|---|---|---|
| DQ095623 | A/Bar-headed Goose/Qinghai/67/05 | 2005 | H5N1 |
| DQ095622 | A/Bar-headed Goose/Qinghai/65/05 | 2005 | H5N1 |
| DQ095621 | A/Bar-headed Goose/Qinghai/12/05 | 2005 | H5N1 |
| DQ095620 | A/Bar-headed Goose/Qinghai/05/05 | 2005 | H5N1 |
| DQ095619 | A/Bar-headed Goose/Qinghai/75/05 | 2005 | H5N1 |
| DQ095618 | A/Bar-headed Goose/Qinghai/61/05 | 2005 | H5N1 |
| DQ095617 | A/Bar-headed Goose/Qinghai/67/05 | 2005 | H5N1 |
| DQ095615 | A/Bar-headed Goose/Qinghai/60/05 | 2005 | H5N1 |
| DQ095613 | A/Bar-headed Goose/Qinghai/68/05 | 2005 | H5N1 |
| DQ095612 | A/Bar-headed Goose/Qinghai/59/05 | 2005 | H5N1 |
| DQ095616 | A/Brown-headed Gull/Qinghai/03/05 | 2005 | H5N1 |
| DQ100554 | A/black-headed goose/Qinghai/1/2005 | 2005 | H5N1 |
| DQ100555 | A/black-headed goose/Qinghai/2/2005 | 2005 | H5N1 |
| DQ100556 | A/black-headed gull/Qinghai/1/2005 | 2005 | H5N1 |
| DQ095614 | A/Great Black-headed Gull/Qinghai/67/05 | 2005 | H5N1 |
| DQ100557 | A/great black-headed gull/Qinghai/1/2005 | 2005 | H5N1 |
| AY651368 | A/Ck/ST/4231/2003 | 2003 | H5N1 |

GTGTGACGAATTCc

| | | | |
|---|---|---|---|
| DQ095623 | A/Bar-headed Goose/Qinghai/67/05 | 2005 | H5N1 |
| DQ095622 | A/Bar-headed Goose/Qinghai/65/05 | 2005 | H5N1 |
| DQ095621 | A/Bar-headed Goose/Qinghai/12/05 | 2005 | H5N1 |
| DQ095620 | A/Bar-headed Goose/Qinghai/05/05 | 2005 | H5N1 |
| DQ095619 | A/Bar-headed Goose/Qinghai/75/05 | 2005 | H5N1 |
| DQ095618 | A/Bar-headed Goose/Qinghai/61/05 | 2005 | H5N1 |
| DQ095617 | A/Bar-headed Goose/Qinghai/67/05 | 2005 | H5N1 |
| DQ095615 | A/Bar-headed Goose/Qinghai/60/05 | 2005 | H5N1 |
| DQ095613 | A/Bar-headed Goose/Qinghai/68/05 | 2005 | H5N1 |
| DQ095612 | A/Bar-headed Goose/Qinghai/59/05 | 2005 | H5N1 |
| DQ095616 | A/Brown-headed Gull/Qinghai/03/05 | 2005 | H5N1 |
| DQ100554 | A/black-headed goose/Qinghai/1/2005 | 2005 | H5N1 |
| DQ100555 | A/black-headed goose/Qinghai/2/2005 | 2005 | H5N1 |
| DQ100556 | A/black-headed gull/Qinghai/1/2005 | 2005 | H5N1 |
| DQ095614 | A/Great Black-headed Gull/Qinghai/67/05 | 2005 | H5N1 |
| DQ100557 | A/great black-headed gull/Qinghai/1/2005 | 2005 | H5N1 |
| DQ007623 | A/Anas platyrhynchos/Chany Lake/9/03 | 2003 | H5N3 |
| AY651368 | A/Ck/ST/4231/2003 | 2003 | H5N1 |
| AY531029 | A/Mallard/64650/03 | 2003 | H5N7 |
| AJ305306 | A/chicken/Italy/8/98 | 1998 | H5N2 |
| AF194169 | A/Chicken/Italy/312/97 | 1997 | H5N2 |
| AF194990 | A/Chicken/Italy/367/97 | 1997 | H5N2 |
| AF194992 | A/Chicken/Italy/9097/97 | 1997 | H5N9 |
| AF194991 | A/Guinea Fowl/Italy/330/97 | 1997 | H5N2 |
| ISDN49024 | A/duck/Singapore/3/97 | 1997 | H5N3 |
| AF303057 | A/duck/Singapore/Q-F119-3/97 | 1997 | H5N3 |
| AF046096 | A/Hong Kong/481/97 | 1997 | H5N1 |
| S68489 | A/turkey/England/50-92/91 | 1991 | H5N1 |
| AF082042 | A/Duck/Potsdam/1402-6/86 | 1986 | H5N2 |
| AF082041 | A/Duck/Potsdam/2216-4/84 | 1984 | H5N6 |
| M18450 | A/Duck/Ireland/113/83 | 1983 | H5N8 |
| M18451 | A/Turkey/Ireland/1378/83 | 1983 | H5N8 |
| AF082039 | A/Duck/Hong Kong/698/79 | 1979 | H5N3 |
| AF290443 | A/Duck/Ho Chi Minh/14/78 | 1978 | H5N3 |
| U20475 | A/duck/Hong Kong/342/78 | 1978 | H5N2 |
| AF082038 | A/Duck/Hong Kong/205/77 | 1977 | H5N3 |
| J02160 | A/shearwater/Australia/75 | 1975 | H5N3 |

GTGTGA T GAATTCc

| | | | |
|---|---|---|---|
| AY573917 | A/chicken/Taiwan/1209/03 | 2003 | H5N2 |
| AY497095 | A/chicken/Guatemala/194573/02 | 2002 | H5N2 |
| AY296079 | A/duck/ME/151895-7A/02 | 2002 | H5N2 |
| AY296083 | A/turkey/CA/D0208651-C/02 | 2002 | H5N2 |
| AY497093 | A/chicken/El Salvador/102711-1/01 | 2001 | H5N2 |
| AY497094 | A/chicken/El Salvador/102711-2/01 | 2001 | H5N2 |
| AY296077 | A/duck/NJ/117228-7/01 | 2001 | H5N2 |
| AJ621811 | A/duck/Primorie/2621/01 | 2001 | H5N2 |
| AJ621807 | A/duck/Primorie/2633/01 | 2001 | H5N3 |
| AY296078 | A/unknown/NY/118547-11/01 | 2001 | H5N2 |
| AY296075 | A/unknown/NY/9899-6/01 | 2001 | H5N2 |
| AY296069 | A/avian/NY/31588-2/00 | 2000 | H5N2 |
| AY296070 | A/avian/NY/31588-3/00 | 2000 | H5N2 |
| AY296074 | A/avian/NY/53726/00 | 2000 | H5N2 |
| AY497088 | A/chicken/Gatemala/45511-1/00 | 2000 | H5N2 |
| AY497089 | A/chicken/Guatemala/45511-2/00 | 2000 | H5N2 |
| AY497090 | A/chicken/Guatemala/45511-3/00 | 2000 | H5N2 |
| AY497091 | A/chicken/Guatemala/45511-4/00 | 2000 | H5N2 |
| AY497092 | A/chicken/Guatemala/45511-5/00 | 2000 | H5N2 |
| AY296073 | A/chukkar/NY/51375/00 | 2000 | H5N2 |
| AY296071 | A/duck/NY/44018-1/00 | 2000 | H5N2 |
| AY296072 | A/duck/NY/44018-2/00 | 2000 | H5N2 |
| AY296084 | A/ruddy turnstone/NJ/2242/00 | 2000 | H5N3 |
| AY684894 | A/mallard/Netherlands/3/99 | 1999 | H5N2 |
| AY497081 | A/chicken/Aguascalientes/124-3705/98 | 1998 | H5N2 |
| AY497079 | A/chicken/FO/22066/98 | 1998 | H5N2 |
| AY497083 | A/chicken/Jalisco/229-4592/98 | 1998 | H5N2 |
| AY497082 | A/chicken/Morelos/227-4353/98 | 1998 | H5N2 |
| AY497080 | A/chicken/Morelos/FO22189/98 | 1998 | H5N2 |
| AY497084 | A/chicken/Puebla/231-5284/98 | 1998 | H5N2 |
| AY497086 | A/chicken/Tabasco/234-8289/98 | 1998 | H5N2 |
| AY497085 | A/chicken/VeraCruz/232-6169/98 | 1998 | H5N2 |
| AY497074 | A/chicken/Chiapas/15224/97 | 1997 | H5N2 |
| AY497075 | A/chicken/Chiapas/15405/97 | 1997 | H5N2 |
| AY497076 | A/chicken/Chiapas/15406/97 | 1997 | H5N2 |
| AY497078 | A/chicken/Chiapas/15408/97 | 1997 | H5N2 |
| AF098538 | A/chicken/chiapis/15224/97 | 1997 | H5N1 |
| AY497077 | A/chicken/Mexico/15407/97 | 1997 | H5N2 |
| AY497073 | A/chicken/Mexico/37821-771/96 | 1996 | H5N2 |
| AY497087 | A/chicken/Queretaro/22019-853/96 | 1996 | H5N2 |
| AY497068 | A/chicken/Chiapa/28159-488/95 | 1995 | H5N2 |
| AY497064 | A/chicken/Guanajuato/28159-331/95 | 1995 | H5N2 |
| AY497066 | A/chicken/Hidalgo/28159-460/95 | 1995 | H5N2 |
| AY497071 | A/chicken/Mexico/28159-541/95 | 1995 | H5N2 |
| AY497069 | A/chicken/Michoacan/28159-530/95 | 1995 | H5N2 |
| AY497070 | A/chicken/Morelos/28159-538/95 | 1995 | H5N2 |
| AY497067 | A/chicken/Puebla/28159-474/95 | 1995 | H5N2 |
| L46587 | A/chicken/Queretaro/14588-19/95 | 1995 | H5N2 |
| U79448 | A/Chicken/Queretaro/7653-20/95 | 1995 | H5N2 |
| AY497065 | A/chicken/VeraCruz/28159-398/95 | 1995 | H5N2 |
| U79455 | A/Turkey/Minnesota/10734/95 | 1995 | H5N2 |
| AY497063 | A/chicken/Hidalgo/232/94 | 1994 | H5N2 |
| U37172 | A/Chicken/Hidalgo/26654-1368/94 | 1994 | H5N2 |
| U37181 | A/Chicken/Jalisco/14585-660/94 | 1994 | H5N2 |
| AY497096 | A/chicken/Mexico/232/94 | 1994 | H5N2 |
| U37173 | A/Chicken/Mexico/26654-1374/94 | 1994 | H5N2 |
| U37166 | A/Chicken/Mexico/31381-1/94 | 1994 | H5N2 |
| U37167 | A/Chicken/Mexico/31381-2/94 | 1994 | H5N2 |
| U37176 | A/Chicken/Mexico/31381-3/94 | 1994 | H5N2 |
| U37174 | A/Chicken/Mexico/31381-4/94 | 1994 | H5N2 |
| U37169 | A/Chicken/Mexico/31381-5/94 | 1994 | H5N2 |
| U37175 | A/Chicken/Mexico/31381-6/94 | 1994 | H5N2 |
| U37165 | A/Chicken/Mexico/31381-7/94 | 1994 | H5N2 |
| U37170 | A/Chicken/Mexico/31381-8/94 | 1994 | H5N2 |
| L46585 | A/chicken/Mexico/31381-Avilab/94 | 1994 | H5N2 |
| U37168 | A/Chicken/Mexico/31382-1/94 | 1994 | H5N2 |
| U37179 | A/Chicken/Puebla/14585-622/94 | 1994 | H5N2 |
| U37180 | A/Chicken/Puebla/14586-654/94 | 1994 | H5N2 |
| U37178 | A/Chicken/Puebla/8623-607/94 | 1994 | H5N2 |
| L46586 | A/chicken/Puebla/8623-607/94 | 1994 | H5N2 |
| U37177 | A/Chicken/Puebla/8624-604/94 | 1994 | H5N2 |
| U37182 | A/Chicken/Queretaro/14588-19/94 | 1994 | H5N2 |
| U37171 | A/Chicken/Queretaro/26654-1373/94 | 1994 | H5N2 |
| U05332 | A/chicken/Florida/25717/93 | 1993 | H5N2 |
| U05331 | A/chicken/Pennsylvania/13609/93 | 1993 | H5N2 |
| U28920 | A/Emu/Texas/39442/93 (HP progeny) | 1993 | H5N2 |
| U28919 | A/Emu/Texas/39442/93 (non-HP parent) | 1993 | H5N2 |
| U05330 | A/ruddy turnstone/Delaware/244/91 | 1991 | H5N2 |
| U67783 | A/Mallard/Ohio/556/1987 | 1987 | H5N9 |
| AF082043 | A/Gull/Pennsylvania/4175/83 | 1983 | H5N1 |
| U79449 | A/Duck/Michigan/80 | 1980 | H5N2 |
| AY500365 | A/turkey/England/N28/73 | 1973 | H5N2 |

Table 2A shows a list of isolates containing the A1384G polymorphism found in the Shantou isolate. Only one other H5N1 sequence was detected. The other isolates were again a migratory bird series including a variety of subtypes not found in humans (H2N3, H2N5, H2N9, H6N2, H6N8). In addition, there were several avian H2N2 sequences as well as human H2N2 sequences associated with the 1957 pandemic. In addition, there was one H1N1 human sequence as well as the first influenza virus isolated from a swine in Iowa in 1930.

A Clustal W alignment of representative sequences was also performed, and the A1384G polymorphism was at the end of the longest common region in the gene. Remarkably, a search of the flu database with the 1385A sequences identified 260 HA sequences and all but 6 were H5N1 beginning with the first H5N1 isolated in Asia in 1996. The list had 4 swine H1N1 sequences from 1997 as well as an H5N3 sequence from 1977 and a H6N2 sequence from 1963.

Thus, a single nucleotide change can convert H5N1 into a migratory bird sequence that traces back to human H2N2 from the 1957 pandemic.

TABLE 2A
Probing HA A1384G
TGGAAAATGAGAGg

| Accession | Strain | Seg | Length | Year | Serotype |
|---|---|---|---|---|---|
| AY639405 | A/goose/China/F3/2004 | HA (4) | 1707 | 2004 | H5N1 |
| AY651368 | A/Ck/ST/4231/2003 | HA (4) | 1697 | 2003 | H5N1 |
| AY684893 | A/mallard/Netherlands/13/99 | HA (4) | 1773 | 1999 | H2N2 |
| AY633196 | A/mallard/Alberta/205/98 | HA (4) | 1753 | 1998 | H2N3 |
| AY633228 | A/mallard/Alberta/226/98 | HA (4) | 1753 | 1998 | H2N3 |
| AY633364 | A/pintail/Alberta/22/97 | HA (4) | 1724 | 1997 | H2N9 |
| AY633388 | A/teal/Alberta/16/97 | HA (4) | 1753 | 1997 | H2N9 |
| AY633180 | A/mallard/Alberta/202/96 | HA (4) | 1739 | 1996 | H2N5 |
| AY633236 | A/mallard/Alberta/232/94 | HA (4) | 1712 | 1994 | H6N8 |
| AF457669 | A/Mallard/Alberta/4/94 | HA (4) | 1702 | 1994 | H6N8 |
| AY633300 | A/mallard/Alberta/76/94 | HA (4) | 1720 | 1994 | H6N8 |
| AY633324 | A/pintail/Alberta/155/94 | HA (4) | 1709 | 1994 | H6N8 |
| AY633380 | A/Redhead/Alberta/291/94 | HA (4) | 1710 | 1994 | H6N8 |
| AF457670 | A/Redhead/Alberta/291/94 | HA (4) | 1705 | 1994 | H6N8 |
| L11131 | A/guinea/NJ/3070/91 | HA (4) | 1773 | 1991 | H2N2 |
| L11132 | A/Herring gull/Delaware/677/88 | HA (4) | 1773 | 1988 | H2N8 |
| L11135 | A/mallard/Alberta/353/88 | HA (4) | 1772 | 1988 | H2N3 |
| AF100181 | A/Mallard Duck/Alberta/211/85 | HA (4) | 1653 | 1985 | H6N2 |
| AY633308 | A/mallard/Alberta/98/85 | HA (4) | 1743 | 1985 | H6N2 |
| L11140 | A/Peking duck/Potsdam/1689-4/85 | HA (4) | 1773 | 1985 | H2N3 |
| AY633316 | A/pintail/Alberta/113/85 | HA (4) | 1725 | 1985 | H6N2 |
| S62154 | A/Alma Ata/1417/84 | HA (4) | 1778 | 1984 | H1N1 |
| L11139 | A/mallard/Potsdam/178-4/83 | HA (4) | 1773 | 1983 | H2N2 |
| L11128 | A/duck/Hong Kong/273/78 | HA (4) | 1772 | 1978 | H2N2 |
| AF290441 | A/Pintail Duck/Primorie/695/76 (recomb) | HA (4) | 1717 | 1976 | H2 |
| L11141 | A/Pintail/Praimoric/625/76 | HA (4) | 1772 | 1976 | H2N2 |
| L11129 | A/duck/GDR/72 | HA (4) | 1773 | 1972 | H2N9 |
| L11125 | A/Berkeley/1/68 | HA (4) | 1773 | 1968 | H2N2 |
| L11133 | A/Korea/426/68 | HA (4) | 1773 | 1968 | H2N2 |
| D13579 | A/Izumi/5/65 | HA (4) | 1773 | 1965 | H2N2 |
| D13580 | A/Izumi/5/65 (R) | HA (4) | 1773 | 1965 | H2N2 |
| L11126 | A/Berlin/3/64 | HA (4) | 1773 | 1964 | H2N2 |
| L11134 | A/Krasnodar/101/59 | HA (4) | 1773 | 1959 | H2N2 |
| L20406 | A/Japan/305+/57 | HA (4) | 1773 | 1957 | H2N2 |
| L20407 | A/Japan/305−/57 | HA (4) | 1773 | 1957 | H2N2 |
| J02127 | A/Japan/305/57 | HA (4) | 1773 | 1957 | H2N2 |
| AB056699 | A/Kayano/57 | HA (4) | 1773 | 1957 | H2N2 |
| L20410 | A/Singapore/1/57 | HA (4) | 1773 | 1957 | H2N2 |
| X57492 | A/Swine/Iowa/15/30 | HA (4) | 1701 | 1930 | H1N1 |

TABLE 2B
HA A1384
TGGAAAATGAGAGa

| | | | | | |
|---|---|---|---|---|---|
| ISDN124038 | A/Chicken/Vietnam/NCVD09/2005 | HA (4) | 1707 | 2005 | H5N1 |
| ISDN124037 | A/Chicken/Vietnam/NCVD10/2005 | HA (4) | 1709 | 2005 | H5N1 |
| ISDN124623 | A/Duck/Vietnam/367/2005 | HA (4) | 1733 | 2005 | H5N1 |
| ISDN124044 | A/Duck/Vietnam/NCVD01/2005 | HA (4) | 1710 | 2005 | H5N1 |
| ISDN124040 | A/Duck/Vietnam/NCVD04/2005 | HA (4) | 1727 | 2005 | H5N1 |
| ISDN124142 | A/Duck/Vietnam/NCVD05/2005 | HA (4) | 1729 | 2005 | H5N1 |
| ISDN124035 | A/Duck/Vietnam/NCVD06/2005 | HA (4) | 1707 | 2005 | H5N1 |
| ISDN124032 | A/Duck/Vietnam/NCVD07/2005 | HA (4) | 1726 | 2005 | H5N1 |
| ISDN124030 | A/Duck/Vietnam/NCVD08/2005 | HA (4) | 1724 | 2005 | H5N1 |
| ISDN124042 | A/Muscovy Duck/Vietnam/NCVD02/2005 | HA (4) | 1735 | 2005 | H5N1 |

.
.
.
240 H5N1 isolates from 1997-2005
.
.
.

| | | | | | |
|---|---|---|---|---|---|
| AF098546 | A/Silky Chicken/Hong Kong/p17/97 | HA (4) | 1726 | 1997 | H5N1 |
| AF222028 | A/Swine/Wisconsin/163/97 | HA (4) | 1747 | 1997 | H1N1 |
| AF222029 | A/Swine/Wisconsin/164/97 | HA (4) | 1773 | 1997 | H1N1 |
| AF222030 | A/Swine/Wisconsin/166/97 | HA (4) | 1773 | 1997 | H1N1 |
| AF222031 | A/Swine/Wisconsin/168/97 | HA(4) | 1774 | 1997 | H1N1 |
| AF148678 | A/goose/Guangdong/1/96 | HA (4) | 1707 | 1996 | H5N1 |
| AF144305 | A/Goose/Guangdong/1/96 | HA (4) | 1760 | 1996 | H5N1 |
| S68489 | A/turkey/England/50-92/91 | HA (4) | 1773 | 1991 | H5N1 |
| AF082038 | A/Duck/Hong Kong/205/77 | HA (4) | 1678 | 1977 | H5N3 |
| AY968676 | A/turkey/Canada/63 | HA (4) | 1745 | 1963 | H6N2 |

Table 3 lists sequences matching the probe for G580A in the Shantou 4231 NA gene. There was only one other H5N1 sequence from Guandong. Many of the polymorphisms in the Shantou sequence were shared with the Guangdong sequence, revealing the Gunagdong sequence evolved from the Shantou sequence or a common source. The two swine sequences were swine with the WSN/33 sequence, which was from 1933. All of the sequences were N1, but were not found in recent isolates. One was from the time of the 1957 pandemic, while the others dated back to the early 1930's and included the first human and swine influenza isolates, as well as an isolate from the 1918 pandemic. Table 3 therefore provides an example of polymorphisms that have not been present recently.

TABLE 3
NA G580A
GGCTGTATTa

| Accession | Strain | Seg | Length | Year | Serotype |
|---|---|---|---|---|---|
| AY609314 | A/chicken/Guangdong/174/04 | NA (6) | 1397 | 2004 | H5N1 |
| AY790269 | A/swine/Korea/S10/2004 | NA (6) | 1097 | 2004 | H1N1 |
| AY790291 | A/swine/Korea/S175/2004 | NA (6) | 1097 | 2004 | H1N1 |
| AY651480 | A/Ck/ST/4231/2003 | NA (6) | 1350 | 2003 | H5N1 |
| AF305216 | A/Denver/1/57 | NA (6) | 379 | 1957 | H1N1 |
| X52226 | A/FPV/Rostock/34 | NA (6) | 1393 | 1934 | H7N1 |
| NC_002018 | A/Puerto Rico/8/34 | NA (6) | 1413 | 1934 | H1N1 |
| L25815 | A/NWS/33 | NA (6) | 1409 | 1933 | H1N1 |
| L25816 | A/WS/33 | NA (6) | 1409 | 1933 | H1N1 |
| AF250364 | A/Swine/Iowa/30 ATCC VR-333 | NA (6) | 1410 | 1930 | H1N1 |
| AF250356 | A/Brevig Mission/1/18 | NA (6) | 1410 | 1918 | H1N1 |

Table 4 shows the results of probing T46C in the PB2 gene from the sequence most closely related to the migratory bird sequences at Qinghai Lake. This was from the peregrine falcon sequence from Hong Kong. The only other H5N1 sequences recognized with this probe were a swine sequence from Fujian province from 2003, a duck sequence from 2000, and sequences from the human outbreak in Hong Kong in 1997 (both avian and human).

The other sequences were from a variety of migratory bird sources or sub-types not found in humans, such as H2N5, H3N3, H3N8, H4N1, H4N6, H5N2, H6N1, H6N2, H6N5, H6N8, H7N3, H7N7, H9N2, H13N6, as well as swine isolates of human sero-types H1N1, H3N2, and H3N2.

In contrast, the above probe with 46T identified 103 sequences (81 H5N1, 20 H9N2, 1 H6N1, 1 H3N2).

TABLE 4
PB2 T46C
ATGGAGAGAATAAAAGAAc

| | | | | | |
|---|---|---|---|---|---|
| AY616766 | A/chicken/British Columbia/04 | PB2 (1) | 2341 | 2004 | H7N3 |
| AY646085 | A/chicken/British Columbia/GSC_human_B/04 | PB2 (1) | 2341 | 2004 | H7N3 |
| AY650276 | A/GSC_chicken/British Columbia/04 | PB2 (1) | 2341 | 2004 | H7N3 |
| AY648294 | A/GSC_chicken_B/British Columbia/04 | PB2 (1) | 2341 | 2004 | H7N3 |
| AY651748 | A/peregrine falcon/HK/D0028/2004 | PB2 (1) | 2280 | 2004 | H5N1 |
| AY342413 | A/avian/Netherlands/219/03 | PB2 (1) | 2341 | 2003 | H7N7 |
| AJ620347 | A/chicken/Germany/R28/03 | PB2 (1) | 2330 | 2003 | H7N7 |
| AY862716 | A/chicken/Korea/S15/03 | PB2 (1) | 1075 | 2003 | H9N2 |
| AY342414 | A/chicken/Netherlands/1/03 | PB2 (1) | 2341 | 2003 | H7N7 |
| AY862714 | A/duck/Korea/S13/03 | PB2 (1) | 1203 | 2003 | H9N2 |

.
.
.
81 isolates from 1985-2003
.
.
.

| | | | | | |
|---|---|---|---|---|---|
| M73516 | A/Gull/Astrakhan/227/84 | PB2 (1) | 2341 | 1984 | H13N6 |
| AY633123 | A/mallard/Alberta/743/83 | PB2 (1) | 1443 | 1983 | H9N1 |
| M73522 | A/Seal/Massachusetts/133/82 | PB2 (1) | 2341 | 1982 | H4N5 |
| M73514 | A/Turkey/Minnesota/833/80 | PB2 (1) | 2341 | 1980 | H4N2 |
| M73525 | A/Gull/Maryland/704/77 | PB2 (1) | 2341 | 1977 | H13N6 |
| M73520 | A/Equine/London/1416/73 | PB2 (1) | 2341 | 1973 | H7N7 |
| M73519 | A/Equine/Prague/1/56 | PB2 (1) | 2341 | 1956 | H7N7 |
| M21851 | A/chicken/FPV/Rostock/34 | PB2 (1) | 2341 | 1934 | H7N1 |
| M38291 | A/FPV/Weybridge | PB2 (1) | 2341 | 1934 | H7N7 |
| M73515 | A/Swine/Iowa/15/30 | PB2 (1) | 2341 | 1930 | H1N1 |

Table 5 shows the result of probing G715T in the NA gene. The only H5N1 isolates detected with this probe were from Vietnam and Thailand in 2004 and 2005. Moreover, no other Influenza A sequence was detected. However, the probe detected Influenza B isolates from 1959 to 2002, but failed to identify the most recent Influenza B isolates. All of the sequences were in the NA gene.

TABLE 5
NA G715T
AGTAATGGtC

| | | | | | |
|---|---|---|---|---|---|
| ISDN124150 | A/Chicken/Vietnam/NCVD09/2005 | NA (6) | 1331 | 2005 | H5N1 |
| ISDN124151 | A/Chicken/Vietnam/NCVD10/2005 | NA (6) | 1330 | 2005 | H5N1 |
| ISDN124152 | A/Chicken/Vietnam/NCVD12/2005 | NA (6) | 1331 | 2005 | H5N1 |
| ISDN124624 | A/Duck/Vietnam/367/2005 | NA (6) | 1335 | 2005 | H5N1 |
| ISDN124145 | A/Duck/Vietnam/NCVD08/2005 | NA (6) | 1329 | 2005 | H5N1 |
| AY651441 | A/bird/Thailand/3.1/2004 | NA (6) | 1350 | 2004 | H5N1 |
| AY770992 | A/chicken/Ayutthaya/Thailand/CU-23/04 | NA (6) | 1282 | 2004 | H5N1 |
| DQ017340 | A/chicken/Kamphaengphet-2-01/2004 | NA (6) | 373 | 2004 | H5N1 |
| DQ017311 | A/chicken/Kamphaengphet-2-02/2004 | NA (6) | 371 | 2004 | H5N1 |
| DQ017339 | A/chicken/Kamphaengphet-2-03/2004 | NA (6) | 370 | 2004 | H5N1 |

.
.
.
187 isolates from 1987-2004
.
.
.

| | | | | |
|---|---|---|---|---|
| AB036870 | B/Victoria/2/87 | NA (6) | 1408 | 1987 |
| M30632 | B/Leningrad/179/86 | NA (6) | 1517 | 1986 |
| M30634 | B/Memphis/6/86 | NA (6) | 1517 | 1986 |
| AY581985 | B/Ibaraki/2/85 | NA (6) | 1405 | 1985 |
| M30639 | B/Victoria/3/85 | NA (6) | 1524 | 1985 |
| AY581984 | B/Norway/1/84 | NA (6) | 1408 | 1984 |
| M30638 | B/USSR/100/83 | NA (6) | 1527 | 1983 |
| M30636 | B/Oregon/5/80 | NA (6) | 1527 | 1980 |
| M30637 | B/Singapore/222/79 | NA (6) | 1527 | 1979 |
| M30633 | B/Maryland/59 | NA (6) | 1557 | 1959 |

Table 6 shows the result of probing the PB1 polymorphism T1075C with the 16 nucleotide sequence. The only H5N1 isolates recognized were from Vietnam and Thailand. The other sequences matched were predominantly H3N2 although the H3N2 sequences were recent, most from isolates after 1999. There were a few other human sequences and older migratory bird sequences recognized.

TABLE 6
PB1 T1075C
cTAGGAAAAGGATACA

| | | | | | |
|---|---|---|---|---|---|
| AY651661 | A/bird/Thailand/3.1/2004 | PB1 (2) | 2269 | 2004 | H5N1 |
| AY770994 | A/chicken/Ayutthaya/Thailand/CU-23/04 | PB1 (2) | 2215 | 2004 | H5N1 |
| AY590582 | A/chicken/Nakorn-Patom/Thailand/CU-K2/2004 | PB1 (2) | 2164 | 2004 | H5N1 |
| AY818130 | A/chicken/Vietnam/C58/04 | PB1 (2) | 2274 | 2004 | H5N1 |
| AY651657 | A/Ck/Thailand/1/2004 | PB1 (2) | 1232 | 2004 | H5N1 |
| AY651658 | A/Ck/Thailand/73/2004 | PB1 (2) | 1317 | 2004 | H5N1 |
| AY651659 | A/Ck/Thailand/9.1/2004 | PB1 (2) | 2269 | 2004 | H5N1 |
| AY651668 | A/Ck/Viet Nam/33/2004 | PB1 (2) | 2269 | 2004 | H5N1 |
| AY651669 | A/Ck/Viet Nam/35/2004 | PB1 (2) | 2270 | 2004 | H5N1 |
| AY651670 | A/Ck/Viet Nam/36/2004 | PB1 (2) | 2270 | 2004 | H5N1 |

.
.
.
247 isolates from 1997-2004
.
.
.

| | | | | | |
|---|---|---|---|---|---|
| AF225519 | A/swine/Shizuoka/115/97 | PB1 (2) | 2274 | 1997 | H3N2 |
| AF225520 | A/swine/Shizuoka/119/97 | PB1 (2) | 2274 | 1997 | H3N2 |
| AF225521 | A/swine/Shizuoka/120/97 | PB1 (2) | 2274 | 1997 | H3N2 |
| AF037422 | A/Fukushima/140/96 | PB1 (2) | 2274 | 1996 | H3N2 |
| U71128 | A/Akita/1/94 | PB1 (2) | 2274 | 1994 | H3N2 |
| AF037418 | A/Kitakyushu/159/93 | PB1 (2) | 2274 | 1993 | H3N2 |
| M25936 | A/Memphis/8/88 | PB1 (2) | 2341 | 1988 | H3N2 |
| AY633122 | A/mallard/Alberta/743/83 | PB1 (2) | 1248 | 1983 | H9N1 |
| M25925 | A/Turkey/Minnesota/833/80 | PB1 (2) | 2341 | 1980 | H4N2 |
| M25926 | A/Mallard/New York/6750/78 | PB1 (2) | 2341 | 1978 | H2N2 |

Table 7 shows the result of probing with another PB1 polymorphism, G1857A that was specific for Vietnam and Thailand (V/T) H5N1 isolates. This polymorphisms was in a subset of the H5N1 V/T isolates and was found in human isolates, primarily H3N2. However, the polymorphism was also found in the earliest H3N2 isolates, which were from 1968, the year of the H3N2 pandemic. However, only a subset of the earliest H3N2 isolates had this polymorphism.

TABLE 7
PB1 G1857A
GAAGTCTGCTTa

| | | | | | |
|---|---|---|---|---|---|
| AY651661 | A/bird/Thailand/3.1/2004 | PB1 (2) | 2269 | 2004 | H5N1 |
| AY770994 | A/chicken/Ayutthaya/Thailand/CU-23/04 | PB1 (2) | 2215 | 2004 | H5N1 |
| AY590582 | A/chicken/Nakorn-Patom/Thailand/CU-K2/2004 | PB1 (2) | 2164 | 2004 | H5N1 |
| AY818130 | A/chicken/Vietnam/C58/04 | PB1 (2) | 2274 | 2004 | H5N1 |
| AY651659 | A/Ck/Thailand/9.1/2004 | PB1 (2) | 2269 | 2004 | H5N1 |
| AY651668 | A/Ck/Viet Nam/33/2004 | PB1 (2) | 2269 | 2004 | H5N1 |
| AY651669 | A/Ck/Viet Nam/35/2004 | PB1 (2) | 2270 | 2004 | H5N1 |
| AY651670 | A/Ck/Viet Nam/36/2004 | PB1 (2) | 2270 | 2004 | H5N1 |
| AY651671 | A/Ck/Viet Nam/37/2004 | PB1 (2) | 2270 | 2004 | H5N1 |
| AY651672 | A/Ck/Viet Nam/38/2004 | PB1 (2) | 2270 | 2004 | H5N1 |

.
.
.
286 isolates from 1970-2004
.
.
.

| | | | | | |
|---|---|---|---|---|---|
| AY210282 | A/Taiwan/2/70 | PB1 (2) | 2274 | 1970 | H3N2 |
| AY210274 | A/England/878/69 | PB1 (2) | 2274 | 1969 | H3N2 |
| AJ564806 | A/England/939/69 | PB1 (2) | 2341 | 1969 | H3N2 |
| AY210275 | A/Rio/6/69 | PB1 (2) | 2274 | 1969 | H3N2 |
| AF348172 | A/Hong Kong/1/68 | PB1 (2) | 2341 | 1968 | H3N2 |
| AF348173 | A/Hong Kong/1/68 (clone MA20C) | PB1 (2) | 2341 | 1968 | H3N2 |
| AY210273 | A/HongKong/16/68 | PB1 (2) | 2274 | 1968 | H3N2 |
| J02138 | A/NT/60/68 | PB1 (2) | 2341 | 1968 | H3N2 |
| AY210271 | A/Panama/1/68 | PB1 (2) | 2274 | 1968 | H3N2 |
| AY210272 | A/USSR/039/68 | PB1 (2) | 2274 | 1968 | H3N2 |

Table 8 is much like Table 5. The probe position was slightly further downstream on the NA gene at G767T. The probe was short, which is necessary at times when crossing species barriers because of the large number of polymorphisms. However, the result was striking because like G715T, the polymorphism was only in the V/Y H5N1 isolates including 2005 and Influenza B. Again, the isolates more recent than 2002 were not identified. Two polymorphisms with the same profile establish strong evidence that Influenza B provided these two V/T specific polymorphisms. Influenza B was therefore further confirmed as a parent.

TABLE 8
NA G767T
TAATGGtC

| | | | | | |
|---|---|---|---|---|---|
| ISDN124150 | A/Chicken/Vietnam/NCVD09/2005 | NA (6) | 1331 | 2005 | H5N1 |
| ISDN124151 | A/Chicken/Vietnam/NCVD10/2005 | NA (6) | 1330 | 2005 | H5N1 |
| ISDN124152 | A/Chicken/Vietnam/NCVD12/2005 | NA (6) | 1331 | 2005 | H5N1 |
| ISDN124624 | A/Duck/Vietnam/367/2005 | NA (6) | 1335 | 2005 | H5N1 |
| ISDN124145 | A/Duck/Vietnam/NCVD08/2005 | NA (6) | 1329 | 2005 | H5N1 |
| AY651441 | A/bird/Thailand/3.1/2004 | NA (6) | 1350 | 2004 | H5N1 |
| AY770992 | A/chicken/Ayutthaya/Thailand/CU-23/04 | NA (6) | 1282 | 2004 | H5N1 |
| DQ017340 | A/chicken/Kamphaengphet-2-01/2004 | NA (6) | 373 | 2004 | H5N1 |
| DQ017311 | A/chicken/Kamphaengphet-2-02/2004 | NA (6) | 371 | 2004 | H5N1 |
| DQ017339 | A/chicken/Kamphaengphet-2-03/2004 | NA (6) | 370 | 2004 | H5N1 |

.
.
.
178 isolates from 1987-2004
.
.
.

| | | | | |
|---|---|---|---|---|
| AB036870 | B/Victoria/2/87 | NA (6) | 1408 | 1987 |
| M30632 | B/Leningrad/179/86 | NA (6) | 1517 | 1986 |
| M30634 | B/Memphis/6/86 | NA (6) | 1517 | 1986 |
| AY581985 | B/Ibaraki/2/85 | NA (6) | 1405 | 1985 |
| M30639 | B/Victoria/3/85 | NA (6) | 1524 | 1985 |
| AY581984 | B/Norway/1/84 | NA (6) | 1408 | 1984 |
| M30638 | B/USSR/100/83 | NA (6) | 1527 | 1983 |
| M30636 | B/Oregon/5/80 | NA (6) | 1527 | 1980 |
| M30637 | B/Singapore/222/79 | NA (6) | 1527 | 1979 |
| M30633 | B/Maryland/59 | NA (6) | 1557 | 1959 |

Table 9 shows that the sequence in Shantou Province that was most closely related to the sequences at Qinghai Lake was close because it had migratory bird sequences from all over Asia and Europe.

TABLE 9
ATAATGGAAA AGAACGTcAC TGTTACACAT
T148C
ATAATGGAAAAGAACGTc

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |

AGAACGTc (HA)

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| DQ006283 | A/Anas platyrhynchos/Chany Lake/7/03 | HA (4) | 511 | 2003 | H2 |
| DQ006282 | A/Anas platyrhynchos/Chany Lake/8/03 | HA (4) | 524 | 2003 | H2 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |

GAACGTc (HA)

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AY651371 | A/Ck/YN/374/2004 | HA (4) | 1666 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| DQ006283 | A/Anas platyrhynchos/Chany Lake/7/03 | HA (4) | 511 | 2003 | H2 |
| DQ006282 | A/Anas platyrhynchos/Chany Lake/8/03 | HA (4) | 524 | 2003 | H2 |
| AY676035 | A/chicken/Korea/ES/03 | HA (4) | 1704 | 2003 | H5N1 |

.
.
.
84 isolates from 1957-2003
.
.
.

| | | | | | |
|---|---|---|---|---|---|
| AF270717 | A/Leningrad/134/57 | HA (4) | 1017 | 1957 | H2N2 |
| AF270722 | A/RI/5+/57 | HA (4) | 1017 | 1957 | H2N2 |
| L20408 | A/RI/5+/57 | HA (4) | 1773 | 1957 | H2N2 |
| AF270718 | A/RI/5−/57 | HA (4) | 1017 | 1957 | H2N2 |
| J02154 | A/ri/5−/57 | HA (4) | 367 | 1957 | H2N2 |
| L20409 | A/RI/5−/57 | HA (4) | 1773 | 1957 | H2N2 |
| L11142 | A/Singapore/1/57 | HA (4) | 1773 | 1957 | H2N2 |
| L20410 | A/Singapore/1/57 | HA (4) | 1773 | 1957 | H2N2 |
| AF231354 | reassortant A/JapanxBellamy/57 | HA (4) | 1077 | 1957 | H2N1 |
| AF231355 | reassortant A/JapanxBellamy/57−MA | HA (4) | 1077 | 1957 | H2N1 |

cACTGTTACACAT

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |

cACTGTTA (HA)

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |
| AJ517815 | A/Switzerland/8808/2002 | HA (4) | 1091 | 2002 | H1N1 |

.
.
.
58 isolates from 1986-2001
.
.
.

| | | | | | |
|---|---|---|---|---|---|
| U46943 | A/Swine/Netherlands/12/85 | HA (4) | 981 | 1985 | H1N1 |
| AF091317 | A/Swine/Netherlands/12/85 | HA (4) | 1776 | 1985 | H1N1 |
| AF320061 | A/Swine/Netherlands/12/85 | HA (4) | 1032 | 1985 | H1N1 |
| M26089 | A/gull/Astrakan/227/84 | HA (4) | 1765 | 1984 | H13N6 |
| AF320056 | A/Swine/France/3614/84 | HA (4) | 1032 | 1984 | H1N1 |
| D00839 | A/duck/Hong Kong/196/77 | HA (4) | 1701 | 1977 | H1 |
| D00838 | A/duck/Hong Kong/36/76 | HA (4) | 1260 | 1976 | H1 |
| M21647 | A/Chicken/Germany/N/49 | HA (4) | 1727 | 1949 | H10N7 |
| ISDN13422 | A/Puerto Rico/8/34 | HA (4) | 1775 | 1934 | H1N1 |
| AF389118 | A/Puerto Rico/8/34/Mount Sinai | HA (4) | 1775 | 1934 | H1N1 |

ACGTcACTGT (HA)

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |
| AF116206 | A/Ruddy turnstone/Delaware/142/98 | HA (4) | 1128 | 1998 | H2N8 |
| AF116209 | A/Shorebird/Delaware/24/98 | HA (4) | 1176 | 1998 | H2N1 |
| AF116210 | A/Shorebird/Delaware/111/97 | HA (4) | 1190 | 1997 | H2N1 |
| AF116211 | A/Shorebird/Delaware/138/97 | HA (4) | 1189 | 1997 | H2N1 |
| AF116207 | A/Ruddy turnstone/Delaware/34/93 | HA (4) | 1185 | 1993 | H2N1 |
| AF116208 | A/Ruddy turnstone/Delaware/81/93 | HA (4) | 1180 | 1993 | H2N1 |
| AF116202 | A/Mallard/New York/66861/78 | HA (4) | 1178 | 1978 | H2N3 |
| L11130 | A/Gull/Maryland/19/77 | HA (4) | 1772 | 1977 | H2N8 |
| AY209955 | A/England/1/61 | HA (4) | 1020 | 1961 | H2N2 |
| AY209956 | A/Panama/1/61 | HA (4) | 1020 | 1961 | H2N2 |
| AY209957 | A/SaoPaolo/1/61 | HA (4) | 1020 | 1961 | H2N2 |
| AY209958 | A/Yale/1/61 | HA (4) | 1020 | 1961 | H2N2 |
| AF270721 | A/Ann Arbor/6/60 | HA (4) | 1017 | 1960 | H2N2 |
| AY209954 | A/Philippines/2/60 | HA (4) | 1020 | 1960 | H2N2 |
| L11134 | A/Krasnodar/101/59 | HA (4) | 1773 | 1959 | H2N2 |
| AF270727 | A/Ohio/2/59 | HA (4) | 1017 | 1959 | H2N2 |
| AF270725 | A/Sao Paolo/3/59 | HA (4) | 1017 | 1959 | H2N2 |
| AF270726 | A/Victoria/15681/59 | HA (4) | 1017 | 1959 | H2N2 |
| AF270723 | A/Albany/6/58 | HA (4) | 1017 | 1958 | H2N2 |
| AF270724 | A/Malaya/16/58 | HA (4) | 1017 | 1958 | H2N2 |
| AF270720 | A/Albany/7/57 | HA (4) | 1017 | 1957 | H2N2 |
| AY209952 | A/Chile/13/57 | HA (4) | 1020 | 1957 | H2N2 |
| AF270728 | A/Chile/6/57 | HA (4) | 1017 | 1957 | H2N2 |
| AF270719 | A/Davis/1/57 | HA (4) | 1017 | 1957 | H2N2 |
| AF270716 | A/El Salvador/2/57 | HA (4) | 1017 | 1957 | H2N2 |
| L20406 | A/Japan/305+/57 | HA (4) | 1773 | 1957 | H2N2 |
| L20407 | A/Japan/305−/57 | HA (4) | 1773 | 1957 | H2N2 |
| AY643086 | A/Japan/305/57 | HA (4) | 1662 | 1957 | H2N2 |
| J02127 | A/Japan/305/57 | HA (4) | 1773 | 1957 | H2N2 |
| AY209953 | A/Japan/305/57 | HA (4) | 1020 | 1957 | H2N2 |
| AY643085 | A/Japan/305/57−MA | HA (4) | 1752 | 1957 | H2N2 |
| AY643087 | A/Japan/305/57−MA, ABT-315675 resistant | HA (4) | 1674 | 1957 | H2N2 |
| AB056699 | A/Kayano/57 | HA (4) | 1773 | 1957 | H2N2 |
| AF270722 | A/RI/5+/57 | HA (4) | 1017 | 1957 | H2N2 |
| L20408 | A/RI/5+/57 | HA (4) | 1773 | 1957 | H2N2 |
| L20409 | A/RI/5−/57 | HA (4) | 1773 | 1957 | H2N2 |
| AF270718 | A/RI/5−/57 | HA (4) | 1017 | 1957 | H2N2 |
| J02154 | A/ri/5−/57 | HA (4) | 367 | 1957 | H2N2 |
| L11142 | A/Singapore/1/57 | HA (4) | 1773 | 1957 | H2N2 |
| L20410 | A/Singapore/1/57 | HA (4) | 1773 | 1957 | H2N2 |
| AF231354 | reassortant A/JapanxBellamy/57 | HA (4) | 1077 | 1957 | H2N1 |
| AF231355 | reassortant A/JapanxBellamy/57−MA | HA (4) | 1077 | 1957 | H2N1 |

AATGTGTGAC GAATTCcTCA ATGTGCCGGA
A287C
TGTGACGAATTCc

| | | | | | |
|---|---|---|---|---|---|
| DQ007623 | A/Anas platyrhynchos/Chany Lake/9/03 | HA (4) | 1326 | 2003 | H5N3 |
| AY651368 | A/Ck/ST/4231/2003 | HA (4) | 1697 | 2003 | H5N1 |
| AY531029 | A/Mallarc/64650/03 | HA (4) | 1695 | 2003 | H5N7 |
| AJ305306 | | HA (4) | 1731 | 1998 | H5N2 |
| AF194169 | A/chicken/Italy/312/97 | HA (4) | 1734 | 1997 | H5N2 |
| AF194990 | A/chicken/Italy/367/97 | HA (4) | 1732 | 1997 | H5N2 |
| AF194992 | A/chicken/Italy/9097/97 | HA (4) | 1679 | 1997 | H5N9 |
| ISDN49024 | | HA (4) | 1761 | 1997 | H5N3 |
| AF303057 | A/duck/Singapore/Q-F119-3/97 | HA (4) | 1761 | 1997 | H5N3 |
| AF194991 | A/Guinea Fowl/Italy/330/97 | HA (4) | 1749 | 1997 | H5N2 |
| AF046096 | A/Hong Kong/481/97 | HA (4) | 1741 | 1997 | H5N1 |
| AF084279 | A/Hong Kong/481/97 | HA (4) | 1665 | 1997 | H5N1 |
| S68489 | A/turkey/England/50-92/91 and A/Chicken/Scotland/59 | HA (4) | 1773 | 1991 | H5N1 |
| AF082042 | A/Duck/Potsdam/1402-6/86 | HA (4) | 1700 | 1986 | H5N2 |
| AF082041 | A/Duck/Potsdam/2216-4/84 | HA (4) | 1700 | 1984 | H5N6 |
| M18450 | A/Duck/Ireland/113/83 | HA (4) | 1773 | 1983 | H5N8 |
| M18451 | A/Turkey/Ireland/1378/83 | HA (4) | 1773 | 1983 | H5N8 |
| AF082039 | A/Duck/Hong Kong/698/79 | HA (4) | 1680 | 1979 | H5N3 |
| AF290443 | A/Duck/Ho Chi Minh/14/78 | HA (4) | 1659 | 1978 | H5N3 |
| U20475 | A/duck/Hong Kong/342/78 | HA (4) | 1008 | 1978 | H5N2 |
| AF082038 | A/Duck/Hong Kong/205/77 | HA (4) | 1678 | 1977 | H5N3 |
| J02160 | A/shearwater/Australia/75 | HA (4) | 355 | 1975 | H5N3 |

cTCAATGTGCCGGA

| | | | | | |
|---|---|---|---|---|---|
| AY651368 | A/Ck/ST/4231/2003 | HA (4) | 1697 | 2003 | H5N1 |
| AJ621807 | A/duck/Primorie/2633/01 | HA (4) | 1707 | 2001 | H5N3 |
| AJ305306 | A/chicken/Italy/8/98 | HA (4) | 1731 | 1998 | H5N2 |
| AF194169 | A/Chicken/Italy/312/97 | HA (4) | 1734 | 1997 | H5N2 |
| AF194990 | A/Chicken/Italy/367/97 | HA (4) | 1732 | 1997 | H5N2 |
| ISDN49024 | A/duck/Singapore/3/97 | HA (4) | 1761 | 1997 | H5N3 |
| AF303057 | A/duck/Singapore/Q-F119-3/97 | HA (4) | 1761 | 1997 | H5N3 |
| AF194991 | A/Guinea Fowl/Italy/330/97 | HA (4) | 1749 | 1997 | H5N2 |
| AF084279 | A/Hong Kong/481/97 | HA (4) | 1665 | 1997 | H5N1 |
| AF046096 | A/Hong Kong/481/97 | HA (4) | 1741 | 1997 | H5N1 |
| S68489 | A/turkey/England/50-92/91 and A/Chicken/Scotland/59 | HA (4) | 1773 | 1991 | H5N1 |

ACGAATTCcTCAATG

| | | | | | |
|---|---|---|---|---|---|
| DQ007623 | A/Anas platyrhynchos/Chany Lake/9/03 | HA (4) | 1326 | 2003 | H5N3 |
| AY651368 | A/Ck/ST/4231/2003 | HA (4) | 1697 | 2003 | H5N1 |
| AY531029 | A/Mallard/64650/03 | HA (4) | 1695 | 2003 | H5N7 |
| AJ305306 | A/chicken/Italy/8/98 | HA (4) | 1731 | 1998 | H5N2 |
| AF194169 | A/Chicken/Italy/312/97 | HA (4) | 1734 | 1997 | H5N2 |
| AF194990 | A/Chicken/Italy/367/97 | HA (4) | 1732 | 1997 | H5N2 |
| AF194992 | A/Chicken/Italy/9097/97 | HA (4) | 1679 | 1997 | H5N9 |
| ISDN49024 | A/duck/Singapore/3/97 | HA (4) | 1761 | 1997 | H5N3 |
| AF303057 | A/duck/Singapore/Q-F119-3/97 | HA (4) | 1761 | 1997 | H5N3 |
| AF194991 | A/Guinea Fowl/Italy/330/97 | HA (4) | 1749 | 1997 | H5N2 |
| AF084279 | A/Hong Kong/481/97 | HA (4) | 1665 | 1997 | H5N1 |
| AF046096 | A/Hong Kong/481/97 | HA (4) | 1741 | 1997 | H5N1 |
| S68489 | A/turkey/England/50-92/91 and A/Chicken/Scotlandl/59 | HA (4) | 1773 | 1991 | H5N1 |

CCTTTTTCAG AAATGTGGTg TGGCTTATCA
G520A
CCTTTTTCAGAAATGTGGTg

| | | | | | |
|---|---|---|---|---|---|
| AY651368 | A/Ck/ST/4231/2003 | HA (4) | 1697 | 2003 | H5N1 |
| AY531029 | A/Mallard/64650/03 | HA (4) | 1695 | 2003 | H5N7 |
| AY684894 | A/mallard/Netherlands/3/99 | HA (4) | 1767 | 1999 | H5N2 |

gTGGCTTATCA

| | | | | | |
|---|---|---|---|---|---|
| AY651368 | A/Ck/ST/4231/2003 | HA (4) | 1697 | 2003 | H5N1 |
| AJ621807 | A/duck/Primorie/2633/01 | HA (4) | 1707 | 2001 | H5N3 |
| AJ305306 | A/chicken/Italy/8/98 | HA (4) | 1731 | 1998 | H5N2 |
| AF194169 | A/Chicken/Italy/312/97 | HA (4) | 1734 | 1997 | H5N2 |
| AF194990 | A/Chicken/Italy/367/97 | HA (4) | 1732 | 1997 | H5N2 |
| ISDN49024 | A/duck/Singapore/3/97 | HA (4) | 1761 | 1997 | H5N3 |
| AF303057 | A/duck/Singapore/Q-F119-3/97 | HA (4) | 1761 | 1997 | H5N3 |
| AF194991 | A/Guinea Fowl/Italy/330/97 | HA (4) | 1749 | 1997 | H5N2 |
| AF084279 | A/Hong Kong/481/97 | HA (4) | 1665 | 1997 | H5N1 |
| AF046096 | A/Hong Kong/481/97 | HA (4) | 1741 | 1997 | H5N1 |
| S68489 | A/turkey/England/50-92/91 and A/Chicken/Scotland/59 | HA (4) | 1773 | 1991 | H5N1 |

TGGCTTATCA AAAAGgACAG TACATACCCA
A136G
gACAGTACA

| | | | | | |
|---|---|---|---|---|---|
| AY651368 | A/Ck/ST/4231/2003 | HA (4) | 1697 | 2003 | H5N1 |
| AF457667 | A/Ruddyturnstone/Delaware/106/98 | HA (4) | 1704 | 1998 | H6N2 |
| AF457668 | A/Ruddyturnstone/Delaware/106/98 | HA (4) | 1704 | 1998 | H6N2 |

TGGCTTATCAAAAAGg

| | | | | | |
|---|---|---|---|---|---|
| AY651368 | A/Ck/ST/4231/2003 | HA (4) | 1697 | 2003 | H5N1 |
| AY651346 | A/Ck/HK/31.2/2002 | HA (4) | 1701 | 2002 | H5N1 |
| AY651350 | A/Ck/HK/3176.3/2002 | HA (4) | 1677 | 2002 | H5N1 |
| AY651347 | A/Ck/HK/37.4/2002 | HA (4) | 1701 | 2002 | H5N1 |
| AY651345 | A/Gf/HK/38/2002 | HA (4) | 1667 | 2002 | H5N1 |
| AY575880 | A/pheasant/Hong Kong/675.14/02 | HA (4) | 1653 | 2002 | H5N1 |
| AY651348 | A/SCk/HK/YU100/2002 | HA (4) | 1701 | 2002 | H5N1 |
| M18450 | A/Duck/Ireland/113/83 | HA (4) | 1773 | 1983 | H5N8 |

AAAAGgACAG

| | | | | | |
|---|---|---|---|---|---|
| AY338455 | A/avian/Netherlands/127/03 | HA (4) | 1675 | 2003 | H7N7 |
| AY338459 | A/avian/Netherlands/219/03 | HA (4) | 1737 | 2003 | H7N7 |
| AY338457 | A/avian/Netherlands/33/03 | HA (4) | 1737 | 2003 | H7N7 |
| AJ620350 | A/chicken/Germany/R28/03 | HA (4) | 1719 | 2003 | H7N7 |
| AJ704813 | A/chicken/Germany/R28/03 | HA (4) | 1181 | 2003 | H7N7 |
| AY338458 | A/chicken/Netherlands/1/03 | HA (4) | 1737 | 2003 | H7N7 |
| AY651368 | A/Ck/ST/4231/2003 | HA (4) | 1697 | 2003 | H5N1 |
| AY651346 | A/Ck/HK/31.2/2002 | HA (4) | 1701 | 2002 | H5N1 |
| AY651347 | A/Ck/HK/37.4/2002 | HA (4) | 1701 | 2002 | H5N1 |
| AY651345 | A/Gf/HK/38/2002 | HA (4) | 1667 | 2002 | H5N1 |

.
.
.
67 isolates from 1998-2002
.
.
.

| | | | | | |
|---|---|---|---|---|---|
| AF028020 | A/England/268/96 | HA (4) | 1732 | 1996 | H7N7 |
| AF202253 | A/ostrich/South Africa/M320/96 | HA (4) | 1732 | 1996 | H7N7 |
| AJ704799 | A/turkey/Ireland/PV8/95 | HA (4) | 1175 | 1995 | H7N7 |
| AF202241 | A/conure/England/1234/94 | HA (4) | 1766 | 1994 | H7N1 |
| AF202249 | A/conure/England/766/94 | HA (4) | 1711 | 1994 | H7N1 |
| AF202229 | A/fairy bluebird/Singapore/F92/94 | HA (4) | 1732 | 1994 | H7N1 |
| AF202251 | A/parakeet/Netherlands/267497/94 | HA (4) | 1732 | 1994 | H7N1 |
| AF202243 | A/parrot/England/1174/94 | HA (4) | 1732 | 1994 | H7N1 |
| AF497555 | A/magpie-robin/China/28710/93 | HA (4) | 1020 | 1993 | H7 |
| D90306 | A/duck/England/56 | HA (4) | 1698 | 1956 | H11N6 |

AAAGTGGAAG GATaGAGTTC TTCTGGACAA
G754A
AAAGTGGAAGGATa

| | | | | | |
|---|---|---|---|---|---|
| AY651368 | A/Ck/ST/4231/2003 | HA (4) | 1697 | 2003 | H5N1 |
| AY497068 | A/chicken/Chiapa/28159-488/95 | HA (4) | 996 | 1995 | H5N2 |
| U20475 | A/duck/Hong Kong/342/78 | HA (4) | 1008 | 1978 | H5N2 |

aGAGTTCTTCTGGACAA

| | | | | | |
|---|---|---|---|---|---|
| AY639405 | A/goose/China/F3/2004 | HA (4) | 1707 | 2004 | H5N1 |
| AY651368 | A/Ck/ST/4231/2003 | HA (4) | 1697 | 2003 | H5N1 |
| AF046097 | A/Hong Kong/483/97 | HA (4) | 1741 | 1997 | H5N1 |
| AF084280 | A/Hong Kong/483/97 | HA (4) | 1665 | 1997 | H5N1 |
| AF290443 | A/Duck/Ho Chi Minh/14/78 | HA (4) | 1659 | 1978 | H5N3 |
| U20475 | A/duck/Hong Kong/342/78 | HA (4) | 1008 | 1978 | H5N2 |

GAAAAGTGAA TTGGgATATG GTAACTGCAA
A885G
GAAAAGTGAATTGGg

| | | | | | |
|---|---|---|---|---|---|
| AY651368 | A/Ck/ST/4231/2003 | HA (4) | 1697 | 2003 | H5N1 |
| AY059478 | A/Duck/Hong Kong/ww461/2000 | HA (4) | 1704 | 2000 | H5N1 |

gATATGGTAACTGCAA

| | | | | | |
|---|---|---|---|---|---|
| AY651368 | A/Ck/ST/4231/2003 | HA (4) | 1697 | 2003 | H5N1 |
| AY059478 | A/Duck/Hong Kong/ww461/2000 | HA (4) | 1704 | 2000 | H5N1 |

CAAACTCCAA TaGGGGCGAT AAACTCTAGT
G922A
CAAACTCCAATa

| | | | | | |
|---|---|---|---|---|---|
| AY651368 | A/Ck/ST/4231/2003 | HA (4) | 1697 | 2003 | H5N1 |
| AY585363 | A/duck/Guangxi/07/1999 | HA (4) | 1708 | 1999 | H5N1 |
| M18450 | A/Duck/Ireland/113/83 | HA (4) | 1773 | 1983 | H5N8 |
| M18451 | A/Turkey/Ireland/1378/83 | HA (4) | 1773 | 1983 | H5N8 |

aGGGGCGAT AAACTCTAGT

| | | | | | |
|---|---|---|---|---|---|
| AY651368 | A/Ck/ST/4231/2003 | HA (4) | 1697 | 2003 | H5N1 |
| AY585363 | A/duck/Guangxi/07/1999 | HA (4) | 1708 | 1999 | H5N1 |

TGTGAAATCA AgCAGATTAG TCCTTGCGAC
A1002G

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY639405 | A/goose/China/F3/2004 | HA (4) | 1707 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |

GAAATCAAg (HA)

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY639405 | A/goose/China/F3/2004 | HA (4) | 1707 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |
| AY063229 | A/Ecuador/2625/01 | HA (4) | 1032 | 2001 | H1N1 |
| AY590822 | A/swine/Gent/108/01 | HA (4) | 1068 | 2001 | H1N2 |
| ISDN13375 | A/SOUTH AFRICA/214/1999 | HA (4) | 979 | 1999 | H1N1 |
| ISDNAU0007 | A/AUCKLAND/18/98 | HA (4) | 848 | 1998 | H1N1 |
| AF072392 | A/Chicken/New York/3202-7/96 | HA (4) | 957 | 1996 | H7N2 |
| AB043492 | A/Kamata/69/96 | HA (4) | 1032 | 1996 | H1N1 |
| AB117207 | A/Nagano/6059/1996 | HA (4) | 981 | 1996 | H1N1 |
| U03719 | A/Swine/Quebec City/5393/91 | HA (4) | 1078 | 1991 | H1 |
| L20112 | A/Singapore/11/90 (egg isolate) | HA (4) | 1032 | 1990 | H1N1 |
| M59324 | A/Ohio/101/83 (isolate A) | HA (4) | 1032 | 1983 | H1N1 |
| M59325 | A/Ohio/101/83 (isolate C) | HA (4) | 1032 | 1983 | H1N1 |
| M59326 | A/Ohio/101/83 (isolate D) | HA (4) | 1032 | 1983 | H1N1 |
| M59327 | A/Ohio/101/83 (isolate F) | HA (4) | 1029 | 1983 | H1N1 |
| M59328 | A/Ohio/201/83 | HA (4) | 1029 | 1983 | H1N1 |
| J02106 | A/duck/New York/12/78 | HA (4) | 332 | 1978 | H11N6 |
| L11137 | A/mallard/NY/6750/78 | HA (4) | 1773 | 1978 | H2N2 |

gCAGATTAGTCCTTGCGAC

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY639405 | A/goose/China/F3/2004 | HA (4) | 1707 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |

ATGGGTACCA CCATAGCAAc GAGCAGGGGA
T1150C
ATGGGTACCA CCATAGCAAc

| | | | | | |
|---|---|---|---|---|---|
| AY651333 | A/Viet Nam/1194/2004 | HA (4) | 1696 | 2004 | H5N1 |
| AY526745 | A/Viet Nam/1196/04 | HA (4) | 303 | 2004 | H5N1 |
| AY651368 | A/Ck/ST/4231/2003 | HA (4) | 1697 | 2003 | H5N1 |
| AJ305306 | A/chicken/Italy/8/98 | HA (4) | 1731 | 1998 | H5N2 |
| AF194169 | A/Chicken/Italy/312/97 | HA (4) | 1734 | 1997 | H5N2 |
| AF194990 | A/Chicken/Italy/367/97 | HA (4) | 1732 | 1997 | H5N2 |
| AF194992 | A/Chicken/Italy/9097/97 | HA (4) | 1679 | 1997 | H5N9 |
| ISDN49024 | A/duck/Singapore/3/97 | HA (4) | 1761 | 1997 | H5N3 |
| AF303057 | A/duck/Singapore/Q-F119-3/97 | HA (4) | 1761 | 1997 | H5N3 |
| AF194991 | A/Guinea Fowl/Italy/330/97 | HA (4) | 1749 | 1997 | H5N2 |
| S68489 | A/turkey/England/50-92/91 and A/Chicken/Scotland/59 | HA (4) | 1773 | 1991 | H5N1 |
| AF082042 | A/Duck/Potsdam/1402-6/86 | HA (4) | 1700 | 1986 | H5N2 |
| AF290443 | A/Duck/Ho Chi Minh/14/78 | HA (4) | 1659 | 1978 | H5N3 |
| AF082038 | A/Duck/Hong Kong/205/77 | HA (4) | 1678 | 1977 | H5N3 |

cGAGCAGGGGA

| | | | | | |
|---|---|---|---|---|---|
| AY651333 | A/Viet Nam/1194/2004 | HA (4) | 1696 | 2004 | H5N1 |
| AY526745 | A/Viet Nam/1196/04 | HA (4) | 303 | 2004 | H5N1 |
| DQ007623 | A/Anas platyrhynchos/Chany Lake/9/03 | HA (4) | 1326 | 2003 | H5N3 |
| AY651368 | A/Ck/ST/4231/2003 | HA (4) | 1697 | 2003 | H5N1 |
| AY531029 | A/Mallard/64650/03 | HA (4) | 1695 | 2003 | H5N7 |
| AY684894 | A/mallard/Netherlands/3/99 | HA (4) | 1767 | 1999 | H5N2 |
| AF194992 | A/Chicken/Italy/9097/97 | HA (4) | 1679 | 1997 | H5N9 |
| ISDN49024 | A/duck/Singapore/3/97 | HA (4) | 1761 | 1997 | H5N3 |
| AF303057 | A/duck/Singapore/Q-F119-3/97 | HA (4) | 1761 | 1997 | H5N3 |
| S68489 | A/turkey/England/50-92/91 and A/Chicken/Scotland/59 | HA (4) | 1773 | 1991 | H5N1 |
| AF082042 | A/Duck/Potsdam/1402-6/86 | HA (4) | 1700 | 1986 | H5N2 |
| AF082041 | A/Duck/Potsdam/2216-4/84 | HA (4) | 1700 | 1984 | H5N6 |
| M18450 | A/Duck/Ireland/113/83 | HA (4) | 1773 | 1983 | H5N8 |
| M18451 | A/Turkey/Ireland/1378/83 | HA (4) | 1773 | 1983 | H5N8 |
| AF082039 | A/Duck/Hong Kong/698/79 | HA (4) | 1680 | 1979 | H5N3 |
| AF082038 | A/Duck/Hong Kong/205/77 | HA (4) | 1678 | 1977 | H5N3 |
| AY500365 | A/turkey/England/N28/73 | HA (4) | 1720 | 1973 | H5N2 |
| U20460 | A/tern/South Africa/61 | HA (4) | 1779 | 1961 | H5N3 |

ACTTAGAAAG GAGAATAGAa AATTTAAACA
G1300A
ACTTAGAAAG GAGAATAGAa

| | | | | | |
|---|---|---|---|---|---|
| AY609312 | A/chicken/Guangdong/174/04 | HA (4) | 1779 | 2004 | H5N1 |
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |
| AY575875 | A/chicken/Hong Kong/31.4/02 | HA (4) | 1710 | 2002 | H5N1 |
| AY575879 | A/chicken/Hong Kong/409.1/02 | HA (4) | 1692 | 2002 | H5N1 |
| AY575878 | A/chicken/Hong Kong/96.1/02 | HA (4) | 1712 | 2002 | H5N1 |
| AY585362 | A/duck/Guangdong/22/2002 | HA (4) | 1708 | 2002 | H5N1 |
| AF509026 | A/Chicken/Hong Kong/822.1/01 | HA (4) | 1375 | 2001 | H5N1 |
| AF509028 | A/Chicken/Hong Kong/830.2/01 | HA (4) | 1529 | 2001 | H5N1 |
| AF509029 | A/Chicken/Hong Kong/858.3/01 | HA (4) | 1554 | 2001 | H5N1 |
| AF509030 | A/Chicken/Hong Kong/867.1/01 | HA (4) | 1406 | 2001 | H5N1 |
| AF509033 | A/Chicken/Hong Kong/876.1/01 | HA (4) | 1537 | 2001 | H5N1 |
| AF509031 | A/Chicken/Hong Kong/879.1/01 | HA (4) | 1668 | 2001 | H5N1 |
| AF509034 | A/Chicken/Hong Kong/891.1/01 | HA (4) | 1659 | 2001 | H5N1 |
| AF509035 | A/Chicken/Hong Kong/893.2/01 | HA (4) | 1497 | 2001 | H5N1 |
| AF509024 | A/Chicken/Hong Kong/SF219/01 | HA (4) | 1654 | 2001 | H5N1 |
| AY221529 | A/Chicken/Hong Kong/YU562/01 | HA (4) | 1705 | 2001 | H5N1 |
| AF509017 | A/Chicken/Hong Kong/YU562/01 | HA (4) | 1656 | 2001 | H5N1 |
| AF509018 | A/Chicken/Hong Kong/YU563/01 | HA (4) | 1656 | 2001 | H5N1 |
| AY221528 | A/Chicken/Hong Kong/YU822.2/01 | HA (4) | 1705 | 2001 | H5N1 |
| AY221527 | A/Chicken/Hong Kong/YU822.2/01-MB | HA (4) | 1705 | 2001 | H5N1 |
| AY585372 | A/duck/Fujian/17/2001 | HA (4) | 1708 | 2001 | H5N1 |
| AY585364 | A/duck/Guangxi/22/2001 | HA (4) | 1708 | 2001 | H5N1 |
| AY585365 | A/duck/Guangxi/35/2001 | HA (4) | 1708 | 2001 | H5N1 |
| AY075033 | A/Duck/Hong Kong/380.5/2001 | HA (4) | 1748 | 2001 | H5N1 |
| AF509039 | A/Duck/Hong Kong/646.3/01 | HA (4) | 1440 | 2001 | H5N1 |
| AY585367 | A/duck/Shanghai/13/2001 | HA (4) | 1708 | 2001 | H5N1 |
| AF509037 | A/Goose/Hong Kong/ww100/01 | HA (4) | 1650 | 2001 | H5N1 |
| ISDN38260 | A/Goose/Vietnam/113/2001 | HA (4) | 1637 | 2001 | H5N1 |
| ISDN38261 | A/Goose/Vietnam/324/2001 | HA (4) | 1637 | 2001 | H5N1 |
| AF509023 | A/Pigeon/Hong Kong/SF215/01 | HA (4) | 1674 | 2001 | H5N1 |
| AF509022 | A/Quail/Hong Kong/SF203/01 | HA (4) | 1635 | 2001 | H5N1 |
| AF509021 | A/Silky Chicken/Hong Kong/SF189/01 | HA (4) | 1674 | 2001 | H5N1 |
| AY059481 | A/Duck/Hong Kong/2986.1/2000 | HA (4) | 1704 | 2000 | H5N1 |
| AY075030 | A/Goose/Hong Kong/3014.5/2000 | HA (4) | 1748 | 2000 | H5N1 |
| AY059482 | A/Goose/Hong Kong/3014.8/2000 | HA (4) | 1704 | 2000 | H5N1 |
| AF082043 | A/Gull/Pennsylvania/4175/83 | HA (4) | 1700 | 1983 | H5N1 |
| U79456 | A/Turkey/Wisconsin/68 | HA (4) | 1647 | 1968 | H5N9 |
| M30122 | A/turkey/Ontario/7732/66 | HA (4) | 1770 | 1966 | H5N9 |

cGAGCAGGGGA

| | | | | | |
|---|---|---|---|---|---|
| AB188824 | A/chicken/Kyoto/3/2004 | HA (4) | 1704 | 2004 | H5N1 |
| AB188816 | A/chicken/Oita/8/2004 | HA (4) | 1704 | 2004 | H5N1 |
| AB166862 | A/chicken/Yamaguchi/7/2004 | HA (4) | 1704 | 2004 | H5N1 |
| AB189053 | A/crow/Kyoto/53/2004 | HA (4) | 1704 | 2004 | H5N1 |
| AB189061 | A/crow/Osaka/102/2004 | HA (4) | 1704 | 2004 | H5N1 |
| AY854190 | A/duck/Shandong/093/2004 | HA (4) | 1779 | 2004 | H5N1 |
| AY639405 | A/goose/China/F3/2004 | HA (4) | 1707 | 2004 | H5N1 |
| AY676035 | A/chicken/Korea/ES/03 | HA (4) | 1704 | 2003 | H5N1 |
| AY651368 | A/Ck/ST/4231/2003 | HA (4) | 1697 | 2003 | H5N1 |
| AY676036 | A/duck/Korea/ESD1/03 | HA (4) | 1704 | 2003 | H5N1 |

.
.
.
64 isolates from 2000-2002
.
.
.

| | | | | | |
|---|---|---|---|---|---|
| AF501235 | A/duck/Shanghai/1/2000 | HA (4) | 1729 | 2000 | H5 |
| AY075030 | A/Goose/Hong Kong/3014.5/2000 | HA (4) | 1748 | 2000 | H5N1 |
| AY059482 | A/Goose/Hong Kong/3014.8/2000 | HA (4) | 1704 | 2000 | H5N1 |
| AF398417 | A/Goose/Hong Kong/385.3/2000 | HA (4) | 1704 | 2000 | H5N1 |
| AF398418 | A/Goose/Hong Kong/385.5/2000 | HA (4) | 1704 | 2000 | H5N1 |
| AY059474 | A/Goose/Hong Kong/ww26/2000 | HA (4) | 1704 | 2000 | H5N1 |
| AY059475 | A/Goose/Hong Kong/ww28/2000 | HA (4) | 1704 | 2000 | H5N1 |
| AY059480 | A/Goose/Hong Kong/ww491/2000 | HA (4) | 1704 | 2000 | H5N1 |
| AF216721 | A/Environment/Hong Kong/437-6/99 | HA (4) | 1741 | 1999 | H5N1 |
| Z46397 | A/England/286/93 | HA (4) | 1041 | 1993 | H3N2 |

TGGAAAATGA GAGgACTCTA GACTTTCATG
A1384G
TGGAAAATGA GAGg

| | | | | | |
|---|---|---|---|---|---|
| AY639405 | A/goose/China/F3/2004 | HA (4) | 1707 | 2004 | H5N1 |
| AY651368 | A/Ck/ST/4231/2003 | HA (4) | 1697 | 2003 | H5N1 |
| AY684893 | A/mallard/Netherlands/13/99 | HA (4) | 1773 | 1999 | H2N2 |
| AY633196 | A/mallard/Alberta/205/98 | HA (4) | 1753 | 1998 | H2N3 |
| AY633228 | A/mallard/Alberta/226/98 | HA (4) | 1753 | 1998 | H2N3 |
| AY633364 | A/pintail/Alberta/22/97 | HA (4) | 1724 | 1997 | H2N9 |
| AY633388 | A/teal/Alberta/16/97 | HA (4) | 1753 | 1997 | H2N9 |
| AY633180 | A/mallard/Alberta/202/96 | HA (4) | 1739 | 1996 | H2N5 |
| AY633236 | A/mallard/Alberta/232/94 | HA (4) | 1712 | 1994 | H6N8 |
| AF457669 | A/Mallard/Alberta/4/94 | HA (4) | 1702 | 1994 | H6N8 |
| AY633300 | A/mallard/Alberta/76/94 | HA (4) | 1720 | 1994 | H6N8 |
| AY633324 | A/pintail/Alberta/155/94 | HA (4) | 1709 | 1994 | H6N8 |
| AY633380 | A/Redhead/Alberta/291/94 | HA (4) | 1710 | 1994 | H6N8 |
| AF457670 | A/Redhead/Alberta/291/94 | HA (4) | 1705 | 1994 | H6N8 |
| L11131 | A/guinea/NJ/3070/91 | HA (4) | 1773 | 1991 | H2N2 |
| L11132 | A/Herring gull/Delaware/677/88 | HA (4) | 1773 | 1988 | H2N8 |
| L11135 | A/mallard/Alberta/353/88 | HA (4) | 1772 | 1988 | H2N3 |
| AF100181 | A/Mallard Duck/Alberta/211/85 | HA (4) | 1653 | 1985 | H6N2 |
| AY633308 | A/mallard/Alberta/98/85 | HA (4) | 1743 | 1985 | H6N2 |
| L11140 | A/Peking duck/Potsdam/1689-4/85 | HA (4) | 1773 | 1985 | H2N3 |
| AY633316 | A/pintail/Alberta/113/85 | HA (4) | 1725 | 1985 | H6N2 |
| S62154 | A/Alma Ata/1417/84 | HA (4) | 1778 | 1984 | H1N1 |
| L11139 | A/mallard/Potsdam/178-4/83 | HA (4) | 1773 | 1983 | H2N2 |
| L11128 | A/duck/Hong Kong/273/78 | HA (4) | 1772 | 1978 | H2N2 |
| AF290441 | A/Pintail Duck/Primorie/695/76 (recomb) | HA (4) | 1717 | 1976 | H2 |
| AF290439 | A/Pintail Duck/Primorie/695/76 (recomb) | HA (4) | 1717 | 1976 | H2 |
| AF290440 | A/Pintail Duck/Primorie/695/76 (recomb) | HA (4) | 1717 | 1976 | H2 |
| AF290442 | A/Pintail Duck/Primorie/695/76 (recomb) | HA (4) | 1717 | 1976 | H2 |
| L11141 | A/Pintail/Praimoric/625/76 | HA (4) | 1772 | 1976 | H2N2 |
| L11129 | A/duck/GDR/72 | HA (4) | 1773 | 1972 | H2N9 |
| L11125 | A/Berkeley/1/68 | HA (4) | 1773 | 1968 | H2N2 |
| L11133 | A/Korea/426/68 | HA (4) | 1773 | 1968 | H2N2 |
| D13579 | A/Izumi/5/65 | HA (4) | 1773 | 1965 | H2N2 |
| D13580 | A/Izumi/5/65 (R) | HA (4) | 1773 | 1965 | H2N2 |
| L11126 | A/Berlin/3/64 | HA (4) | 1773 | 1964 | H2N2 |
| L11134 | A/Krasnodar/101/59 | HA (4) | 1773 | 1959 | H2N2 |
| L20406 | A/Japan/305+/57 | HA (4) | 1773 | 1957 | H2N2 |
| L20407 | A/Japan/305−/57 | HA (4) | 1773 | 1957 | H2N2 |
| J02127 | A/Japan/305/57 | HA (4) | 1773 | 1957 | H2N2 |
| AY643086 | A/Japan/305/57 | HA (4) | 1662 | 1957 | H2N2 |
| AY643085 | A/Japan/305/57-MA | HA (4) | 1752 | 1957 | H2N2 |
| AY643087 | A/Japan/305/57-MA, ABT-315675 resistant | HA (4) | 1674 | 1957 | H2N2 |
| AB056699 | A/Kayano/57 | HA (4) | 1773 | 1957 | H2N2 |
| L20408 | A/RI/5+/57 | HA (4) | 1773 | 1957 | H2N2 |
| L20409 | A/RI/5−/57 | HA (4) | 1773 | 1957 | H2N2 |
| L11142 | A/Singapore/1/57 | HA (4) | 1773 | 1957 | H2N2 |
| L20410 | A/Singapore/1/57 | HA (4) | 1773 | 1957 | H2N2 |
| X57492 | A/Swine/Iowa/15/30 | HA (4) | 1701 | 1930 | H1N1 |

gACTCTAG (HA)

| | | | | | |
|---|---|---|---|---|---|
| AY639405 | A/goose/China/F3/2004 | HA (4) | 1707 | 2004 | H5N1 |
| AY651368 | A/Ck/ST/4231/2003 | HA (4) | 1697 | 2003 | H5N1 |
| AJ344021 | A/swine/Cotes d'Armor/1455/99 | HA (4) | 1721 | 1999 | H1N1 |
| AJ412712 | A/swine/Cotes d'Armor/1488/99 | HA (4) | 1679 | 1999 | H1N1 |
| AJ344016 | A/swine/Italy/1511/98 | HA (4) | 1677 | 1998 | H1N1 |
| AJ344019 | A/swine/Italy/1498-2/97 | HA (4) | 1671 | 1997 | H1N1 |
| AJ344017 | A/swine/Italy/1509-6/97 | HA (4) | 1671 | 1997 | H1N1 |
| AJ344008 | A/swine/Italy/1456-1/96 | HA (4) | 1661 | 1996 | H1N1 |
| U20468 | A/rhea/North Carolina/39482/93 | HA (4) | 1107 | 1993 | H7N1 |
| U72669 | A/Swine/Schleswig-Holstein/1/93 | HA (4) | 1778 | 1993 | H1N1 |
| U72667 | A/Swine/England/195852/92 | HA (4) | 1777 | 1992 | H1N1 |
| Z46434 | A/swine/Germany/8533/91 | HA (4) | 1730 | 1991 | H1N1 |
| AF497551 | A/Turkey/Minnesota/38429/88 | HA (4) | 1049 | 1988 | H7 |
| AF091315 | A/Swine/Italy-Virus/671/87 | HA (4) | 1777 | 1987 | H1N1 |
| AF091317 | A/Swine/Netherlands/12/85 | HA (4) | 1776 | 1985 | H1N1 |
| AF082041 | A/Duck/Potsdam/2216-4/84 | HA (4) | 1700 | 1984 | H5N6 |
| AF091316 | A/Swine/Belgium/1/83 | HA (4) | 1778 | 1983 | H1N1 |
| Z30276 | A/swine/Germany/2/81 | HA (4) | 1730 | 1981 | H1N1 |
| AF497559 | A/turkey/Ontario/81 | HA (4) | 1011 | 1981 | H7 |
| AF091312 | A/duck/Australia/749/80 | HA (4) | 1777 | 1980 | H1N1 |
| AF091314 | A/Swine/Netherlands/3/80 | HA (4) | 1778 | 1980 | H1N1 |
| U20466 | A/turkey/Minnesota/1237/80 | HA (4) | 1089 | 1980 | H7N3 |
| AF497553 | A/pintail/Alberta/21/79 | HA (4) | 1045 | 1979 | H7 |
| AF091313 | A/duck/Bavaria/1/77 | HA (4) | 1777 | 1977 | H1N1 |
| D00839 | A/duck/Hong Kong/196/77 | HA (4) | 1701 | 1977 | H1 |
| AF497554 | A/Duck/Alberta/49/76 | HA (4) | 1032 | 1976 | H7 |
| AY500365 | A/turkey/England/N28/73 | HA (4) | 1720 | 1973 | H5N2 |

AATGCAAAGG AGCTtGGTAA CGGTTGTTTC
G1465T
AATGCAAAGGAGCTt

| | | | | | |
|---|---|---|---|---|---|
| ISDN124044 | A/Duck/Vietnam/NCVD01/2005 | HA (4) | 1710 | 2005 | H5N1 |
| AY651368 | A/Ck/ST/4231/2003 | HA (4) | 1697 | 2003 | H5N1 |
| AY651359 | A/grey heron/HK/861.1/2002 | HA (4) | 1696 | 2002 | H5N1 |

tGGTAACGGTTGTTTC

| | | | | | |
|---|---|---|---|---|---|
| ISDN124044 | A/Duck/Vietnam/NCVD01/2005 | HA (4) | 1710 | 2005 | H5N1 |
| AY651368 | A/Ck/ST/4231/2003 | HA (4) | 1697 | 2003 | H5N1 |
| AY651359 | A/grey heron/HK/861.1/2002 | HA (4) | 1696 | 2002 | H5N1 |

CAGTGGCGAG cTCCCTAGCA CTGGCAATCA
T1651C
CAGTGGCGAG c

| | | | | | |
|---|---|---|---|---|---|
| AY651333 | A/Viet Nam/1194/2004 | HA (4) | 1696 | 2004 | H5N1 |
| AY651368 | A/Ck/ST/4231/2003 | HA (4) | 1697 | 2003 | H5N1 |

cTCCCTAGCA CTGGCAATCA

| | | | | | |
|---|---|---|---|---|---|
| AY651333 | A/Viet Nam/1194/2004 | HA (4) | 1696 | 2004 | H5N1 |
| AY651368 | A/Ck/ST/4231/2003 | HA (4) | 1697 | 2003 | H5N1 |

Table 10 shows the result of probing with a polymorphism in Thailand (A103T in HA). In many instances there were regional polymorphisms. There were quite a few that were found in Vietnam and Thailand, and others specific for Vietnam or Thailand. The highly specific polymorphisms were of interest because they correlated with the H5N1's that can cause fatal infections in humans. The A103T polymorphism is one such polymorphism. All H5N1's with this polymorphism were in Thailand. The polymorphism was traced using a 9 bp sequence. The isolates having exact matches are listed below. The sequence was initially in H2, which is a human H that disappeared in 1968 when H3 replaced H2. However, the sequence then started to appear in H9's. In the late 70's it was in Hong Kong. It was in most H9's from Korea; and Korea had a very distinctive H9. It was also in Japan, which is adjacent to Korea. It appeared in Vietnam as an H9 in 2001. After Korea, it was found in H5 in Thailand. The list below shows all exact matches of this sequence found in the Los Alamos database.

TABLE 10
Sequence = TTGGGAACt
Polymorphism = A103T

| | | | | | |
|---|---|---|---|---|---|
| AY651441 | A/bird/Thailand/3.1/2004 | NA (6) | 1350 | 2004 | H5N1 |
| AY770992 | A/chicken/Ayutthaya/Thailand/CU-23/04 | NA (6) | 1282 | 2004 | H5N1 |
| AY790314 | A/chicken/Korea/S20/2004 | HA (4) | 1104 | 2004 | H9N2 |
| AY790315 | A/chicken/Korea/S22/2004 | HA (4) | 1104 | 2004 | H9N2 |
| AY590567 | A/chicken/Nakorn-Patom/Thailand/CU-K2/2004 | NA (6) | 1237 | 2004 | H5N1 |
| AY660557 | A/chicken/Nakorn-Patom/Thailand/CU-K3/2004 | NA (6) | 1330 | 2004 | H5N1 |
| DQ076202 | A/Chicken/Thailand/73/2004 | NA (6) | 1357 | 2004 | H5N1 |
| AY649383 | A/chicken/Thailand/CH-2/2004 | NA (6) | 1389 | 2004 | H5N1 |
| AY779051 | A/chicken/Thailand/CU-21/2004 | NA (6) | 1304 | 2004 | H5N1 |
| AY651439 | A/Ck/Thailand/1/2004 | NA (6) | 1350 | 2004 | H5N1 |

.
.
.
240 H5N1 isolates from 1966-2004
.
.
.

| | | | | | |
|---|---|---|---|---|---|
| AY209976 | A/Canada/1/66 | HA (4) | 1020 | 1966 | H2N2 |
| AY209977 | A/Panama/1/66 | HA (4) | 1020 | 1966 | H2N2 |
| AF156390 | A/Turkey/California/189/66 | HA (4) | 1661 | 1966 | H9N2 |
| D13579 | A/Izumi/5/65 | HA (4) | 1773 | 1965 | H2N2 |
| D13580 | A/Izumi/5/65 (R) | HA (4) | 1773 | 1965 | H2N2 |
| AY209971 | A/Kumamoto/1/65 | HA (4) | 1020 | 1965 | H2N2 |
| AY209973 | A/Pittsburgh/2/65 | HA (4) | 1020 | 1965 | H2N2 |
| AF270723 | A/Albany/6/58 | HA (4) | 1017 | 1958 | H2N2 |
| L20408 | A/RI/5+/57 | HA (4) | 1773 | 1957 | H2N2 |
| L20409 | A/RI/5−/57 | HA (4) | 1773 | 1957 | H2N2 |

Homologous sequences located at a distance from one another within a virus or viruses (such homologous sequences can be located at distinct loci or at two different sites within the same locus) can transfer with one another during emergence of new viral strains. This process is also likely mediated by recombination. An exemplary observation of such a process was observed when a probe to the PB2 gene was examined across influenza strains, as shown in Table 11.

TABLE 11
PB2 1906 (1997)
Probe Sequence: CCACCTa

| | | | | | |
|---|---|---|---|---|---|
| ISDN124038 | A/Chicken/Vietnam/NCVD09/2005 | HA (4) | 1707 | 2005 | H5N1 |
| ISDN124037 | A/Chicken/Vietnam/NCVD10/2005 | HA (4) | 1709 | 2005 | H5N1 |
| ISDN124623 | A/Duck/Vietnam/367/2005 | HA (4) | 1733 | 2005 | H5N1 |
| ISDN124044 | A/Duck/Vietnam/NCVD01/2005 | HA (4) | 1710 | 2005 | H5N1 |
| ISDN124040 | A/Duck/Vietnam/NCVD04/2005 | HA (4) | 1727 | 2005 | H5N1 |
| ISDN124142 | A/Duck/Vietnam/NCVD05/2005 | HA (4) | 1729 | 2005 | H5N1 |
| ISDN124035 | A/Duck/Vietnam/NCVD06/2005 | HA (4) | 1707 | 2005 | H5N1 |
| ISDN124032 | A/Duck/Vietnam/NCVD07/2005 | HA (4) | 1726 | 2005 | H5N1 |
| ISDN124030 | A/Duck/Vietnam/NCVD08/2005 | HA (4) | 1724 | 2005 | H5N1 |
| ISDN124042 | A/Muscovy Duck/Vietnam/NCVD02/2005 | HA (4) | 1735 | 2005 | H5N1 |

.
.
.
694 isolates from 1976-2005
.
.
.

| | | | | | |
|---|---|---|---|---|---|
| X57491 | A/Swine/Hong Kong/1/74 | HA (4) | 1074 | 1974 | H1N1 |
| M30750 | A/Equine/London/1416/73 | NP (5) | 1565 | 1973 | H7N7 |
| U20472 | A/turkey/Colorado/72 | HA (4) | 234 | 1972 | H5N2 |
| M76607 | A/Swine/Wisconsin/1/67 | NP (5) | 1565 | 1967 | H1N1 |
| AF156390 | A/Turkey/California/189/66 | HA (4) | 1661 | 1966 | H9N2 |
| D90305 | A/turkey/Wisconsin/66 | HA (4) | 1683 | 1966 | H9N2 |
| M22575 | A/Equine/Miami/1/63 | NP (5) | 1565 | 1963 | H3N8 |
| M63763 | A/Swine/Wisconsin/1/61 | NP (5) | 1565 | 1961 | H1N1 |
| M63762 | A/Swine/Wisconsin/1/57 | NP (5) | 1565 | 1957 | H1N1 |
| M63761 | A/Swine/May/54 | NP (5) | 1565 | 1954 | H1N1 |

In Table 11 (above), a seven nucleotide probe was used to identify the source of PB2 G1906A found in H5N1 patients in 1997. This probe contained A1905T in addition to G1906A. It identified 714 sequences in the Los Alamos flu database, and the nits were far from random. All of the 2004 and 2005 hits were H5 in H5N1 isolates. Older isolates contained the sequence in H1 and H9. Several isolates contained the sequence in two genes, H5 and PA or H1 and NP. The two isolates with PB2 G1906A had the sequence in three genes, H5, PA, and PB2. The matches for the probe were present at specific times and in unusual combinations or genes (reassortants) indicating that these probes were markers for sequences that directed assembly of genes in cells infected with two or more distinct viruses.

Additional data showing a similar effect are presented in Table 12:

TABLE 12
HA G473A
GTCACTCAa (HA)

| | | | | | |
|---|---|---|---|---|---|
| ISDN119864 | A/Norway/70/2005 | HA (4) | 1111 | 2005 | H3N2 |
| ISDN110508 | A/Auckland/13/2004 | HA (4) | 1031 | 2004 | H3N2 |
| ISDN110513 | A/Auckland/45/2004 | HA (4) | 1014 | 2004 | H3N2 |
| ISDN110514 | A/Auckland/57/2004 | HA (4) | 1018 | 2004 | H3 |
| ISDN64766 | A/Bangkok/1158/2004 | HA (4) | 1023 | 2004 | H3 |
| ISDN64760 | A/Brisbane/1/2004 | HA (4) | 1021 | 2004 | H3N2 |
| ISDN110521 | A/Brisbane/25/2004 | HA (4) | 1036 | 2004 | H3 |
| ISDN110518 | A/Brisbane/70/2004 | HA (4) | 1028 | 2004 | H3N2 |
| ISDN110647 | A/California/7/2004 (cell-passaged) | HA (4) | 1538 | 2004 | H3 |
| ISDN110648 | A/California/7/2004 (egg-passaged) | HA (4) | 1538 | 2004 | H3 |

.
.
.
213 isolates from 2000-2004
.
.
.

| | | | | | |
|---|---|---|---|---|---|
| AY180836 | A/Quail/Nanchang/12-340/2000 | NA (6) | 1463 | 2000 | H1N1 |
| ISDN33627 | A/NEW CALEDONIA/20/99 | NA (6) | 1409 | 1999 | H1N1 |
| AJ518092 | A/New Caledonia/20/99 | NA (6) | 1459 | 1999 | H1N1 |
| ISD3BF2A773 | A/NANCHANG/4/98 | NA (6) | 1413 | 1998 | H1N1 |
| ISDN20056 | A/NEIMENGGU/36/98 | NA (6) | 1413 | 1998 | H1N1 |
| ISD3BF2A72F | A/HONG KONG/364/97 | NA (6) | 1413 | 1997 | H1N1 |
| AJ518096 | A/Johannesburg/159/97 | NA (6) | 1459 | 1997 | H1N1 |
| ISD3BF2A5CF | A/TAIWAN/1348/97 | NA (6) | 1413 | 1997 | H1N1 |
| ISD3BF2A81C | A/WUHAN/100/97 | NA (6) | 1413 | 1997 | H1N1 |
| AJ518097 | A/Wuhan/371/95 | NA (6) | 1459 | 1995 | H1N1 |

aAATGGAACAA

| | | | | | |
|---|---|---|---|---|---|
| ISDN119864 | A/Norway/70/2005 | HA (4) | 1111 | 2005 | H3N2 |
| ISDN110508 | A/Auckland/13/2004 | HA (4) | 1031 | 2004 | H3N2 |
| ISDN110513 | A/Auckland/45/2004 | HA (4) | 1014 | 2004 | H3N2 |
| ISDN110514 | A/Auckland/57/2004 | HA (4) | 1018 | 2004 | H3 |
| ISDN64766 | A/Bangkok/1158/2004 | HA (4) | 1023 | 2004 | H3 |
| ISDN64760 | A/Brisbane/1/2004 | HA (4) | 1021 | 2004 | H3N2 |
| ISDN110521 | A/Brisbane/25/2004 | HA (4) | 1036 | 2004 | H3 |
| ISDN110518 | A/Brisbane/70/2004 | HA (4) | 1028 | 2004 | H3N2 |
| ISDN110647 | A/California/7/2004 (cell-passaged) | HA (4) | 1538 | 2004 | H3 |
| ISDN110648 | A/California/7/2004 (egg-passaged) | HA (4) | 1538 | 2004 | H3 |

.
.
.
205 isolates from 1980-2004
.
.
.

| | | | | | |
|---|---|---|---|---|---|
| U20466 | A/turkey/Minnesota/1237/80 | HA (4) | 1089 | 1980 | H7N3 |
| AF497553 | A/pintail/Alberta/21/79 | HA (4) | 1045 | 1979 | H7 |
| M19057 | A/Swine/Hong Kong/81/78 | HA (4) | 1647 | 1978 | H3N2 |
| D00932 | A/Duck/Hong Kong/231/77 | HA (4) | 1653 | 1977 | H3 |
| AF497554 | A/Duck/Alberta/49/76 | HA (4) | 1032 | 1976 | H7 |
| D00931 | A/Duck/Hong Kong/64/76 | HA (4) | 1653 | 1976 | H3 |
| D00930 | A/Goose/Hong Kong/10/76 | HA (4) | 1653 | 1976 | H3 |
| D00929 | A/Duck/Hong Kong/7/75 | HA (4) | 1653 | 1975 | H3 |
| M17813 | A/tern/Australia/G70C/75 | NA (6) | 1467 | 1975 | H11N9 |
| M11445 | A/tern/Australia/G70C/75 (recomb) | NA (6) | 1467 | 1975 | H1N9 |

CTCAaAATGG

| | | | | | |
|---|---|---|---|---|---|
| ISDN119864 | A/Norway/70/2005 | HA (4) | 1111 | 2005 | H3N2 |
| ISDN110508 | A/Auckland/13/2004 | HA (4) | 1031 | 2004 | H3N2 |
| ISDN110513 | A/Auckland/45/2004 | HA (4) | 1014 | 2004 | H3N2 |
| ISDN110514 | A/Auckland/57/2004 | HA (4) | 1018 | 2004 | H3 |
| ISDN64766 | A/Bangkok/1158/2004 | HA (4) | 1023 | 2004 | H3 |
| ISDN64760 | A/Brisbane/1/2004 | HA (4) | 1021 | 2004 | H3N2 |
| ISDN110521 | A/Brisbane/25/2004 | HA (4) | 1036 | 2004 | H3 |
| ISDN110518 | A/Brisbane/70/2004 | HA (4) | 1028 | 2004 | H3N2 |
| ISDN110647 | A/California/7/2004 (cell-passaged) | HA (4) | 1538 | 2004 | H3 |
| ISDN110648 | A/California/7/2004 (egg-passaged) | HA (4) | 1538 | 2004 | H3 |

.
.
.
151 isolates from 1977-2004
.
.
.

| | | | | | |
|---|---|---|---|---|---|
| AJ410542 | A/duck/Hong Kong/175/77 | HA (4) | 1701 | 1977 | H6N1 |
| AJ410543 | A/duck/Hong Kong/182/77 | HA (4) | 1701 | 1977 | H6N9 |
| AJ410544 | A/duck/Hong Kong/202/77 | HA (4) | 1701 | 1977 | H6N1 |
| AJ410545 | A/duck/Hong Kong/221/77 | HA (4) | 1701 | 1977 | H6N8 |
| AJ410546 | A/goose/Hong Kong/17/77 | HA (4) | 1701 | 1977 | H6N4 |
| M73771 | A/Duck/Alberta/78/76 | HA (4) | 1765 | 1976 | H3N2 |
| AJ410540 | A/duck/Hong Kong/108/76 | HA (4) | 1701 | 1976 | H6N5 |
| AJ410539 | A/duck/Hong Kong/73/76 | HA (4) | 1701 | 1976 | H6N1 |
| M73772 | A/Duck/Memphis/928/74 | HA (4) | 1765 | 1974 | H3N8 |
| D90303 | A/shearwater/Australia/1/72 | HA (4) | 1701 | 1972 | H6N5 |

In Table 12, probes of H3N2 HA were used to analyze the origins of G473A (numbering using HA sequence of A/Fujian/4/2004). A longer probe using 12 nucleotides upstream from G473A identified the first 99 sequences in the table above. All sequences were from 2004 and 2005, demonstrating that G473A was a newly acquired HA sequence. All of the sero-types were H3 or H3N2. The 2004 series from Nepal were from the spring of 2004, predating the outbreak of California/7 in the fall of 2004.

A shorter sequence using the 8 nucleotides upstream from G473 identified HA and NA sequences. The HA sequences were all H3 with the exception of one H8N4 sequence from a 1984 mallard duck. The remaining sequences were predominantly equine H3N8 sequences from 1963 to 2002. There were also two H3N2 sequences, one from a 1978 Alberta duck, and one from the 1968 Hong Kong pandemic isolate. The probe also identified NA in H1N1 isolates, which were predominantly from Asia between 1995 and 2002.

A probe using the 10 nucleotides downstream from G473A identified the 2004 and 2005 isolates as well as a 1998 isolate from Spain. However, the probe also identified H7N2 and H7N3 isolates from the Americas from 1980 to 2002. H7 is the only avian serotype that has been reported to cause a fatal human infection, in 2003 in the Netherlands during the H7N7 outbreak. Many people culling the birds, as well as their contacts had H7 antibodies although most had mild symptoms ranging from no symptoms to conjunctivitis, to mild flu-like symptoms. The probe also identified H3 prior to 1992 in non-human isolates with serotypes H3N3, H3N8, and swine H3N2. In addition N2 and N9 were detected. N2 was in H7N2 isolates and most of these isolates had the probed sequence in both HA and NA between 1997 and 2002 on the east coast of the US. The 2002 isolates were in New Jersey and Virginia. A government worker involved with culling the 2002 Virginia outbreak was found to have H7N2 antibodies and 2003 serum from a New York resident was also H7N2 positive. The probe also identified a few H9 isolates in 1985 and earlier.

A 10 nucleotide probe with G473A at position 5 detected H6 isolates with N1, N2, N4, N5, N8, N9. including an H6N1 dove in Korea in 2003. The live markets in Korea also had H3N2 dove infections. H6 infections were found as early as 1972 in a migratory shearwater in Australia.

TABLE 13
HA G149A
TGCAGTACCAAACGGAACa

| | | | | | |
|---|---|---|---|---|---|
| ISDN64766 | A/Bangkok/1158/2004 | HA (4) | 1023 | 2004 | H3 |
| ISDN64769 | A/Fiji/185/2004 | HA (4) | 1010 | 2004 | H3 |
| ISDN64772 | A/Macau/103/2004 | HA (4) | 1010 | 2004 | H3N2 |
| ISDN64751 | A/Malaysia/1/2004 | HA (4) | 1011 | 2004 | H3N2 |
| ISDN110605 | A/Malaysia/1875/2004 | HA (4) | 1022 | 2004 | H3N2 |
| ISDN64763 | A/Malaysia/452/2004 | HA (4) | 1042 | 2004 | H3 |
| ISDN69020 | A/Malaysia/661/2004 | HA (4) | 1020 | 2004 | H3 |
| AY945273 | A/Nepal/1723/2004 | HA (4) | 1000 | 2004 | H3N2 |
| AY945271 | A/Nepal/1729/2004 | HA (4) | 1000 | 2004 | H3N2 |
| AY619977 | A/Swine/Ontario/42729A/01 | HA (4) | 1701 | 2001 | H3N3 |
| AY619969 | A/Swine/Ontario/K01477/01 | HA (4) | 1701 | 2001 | H3N3 |
| ISDN13379 | A/Sydney/118/2000 | HA (4) | 1103 | 2000 | H3N2 |
| AY633172 | A/mallard/Alberta/199/99 | HA (4) | 1717 | 1999 | H3N6 |
| AY633372 | A/pintail/Alberta/37/99 | HA (4) | 1743 | 1999 | H3N8 |

aATAGTGAAAAC

| | | | | | |
|---|---|---|---|---|---|
| ISDN64766 | A/Bangkok/1158/2004 | HA (4) | 1023 | 2004 | H3 |
| ISDN64769 | A/Fiji/185/2004 | HA (4) | 1010 | 2004 | H3 |
| ISDN64772 | A/Macau/103/2004 | HA (4) | 1010 | 2004 | H3N2 |
| ISDN64751 | A/Malaysia/1/2004 | HA (4) | 1011 | 2004 | H3N2 |
| ISDN110605 | A/Malaysia/1875/2004 | HA (4) | 1022 | 2004 | H3N2 |
| ISDN64763 | A/Malaysia/452/2004 | HA (4) | 1042 | 2004 | H3 |
| ISDN69020 | A/Malaysia/661/2004 | HA (4) | 1020 | 2004 | H3 |
| AY945273 | A/Nepal/1723/2004 | HA (4) | 1000 | 2004 | H3N2 |
| AY945271 | A/Nepal/1729/2004 | HA (4) | 1000 | 2004 | H3N2 |
| AY862607 | A/chicken/Korea/S6/03 | HA (4) | 1716 | 2003 | H3N2 |
| AY862612 | A/dove/Korea/S11/03 | HA (4) | 1415 | 2003 | H3N2 |
| AY862611 | A/duck/Korea/S10/03 | HA (4) | 1410 | 2003 | H3N2 |
| AY862608 | A/duck/Korea/S7/03 | HA (4) | 1412 | 2003 | H3N2 |
| AY862609 | A/duck/Korea/S8/03 | HA (4) | 1410 | 2003 | H3N2 |
| AY862610 | A/duck/Korea/S9/03 | HA (4) | 1412 | 2003 | H3N2 |
| AY619977 | A/Swine/Ontario/42729A/01 | HA (4) | 1701 | 2001 | H3N3 |
| AY619969 | A/Swine/Ontario/K01477/01 | HA (4) | 1701 | 2001 | H3N3 |
| AY633172 | A/mallard/Alberta/199/99 | HA (4) | 1717 | 1999 | H3N6 |
| AY633372 | A/pintail/Alberta/37/99 | HA (4) | 1743 | 1999 | H3N8 |
| L31949 | A/Seal/Massachusetts/3911/92 | HA (4) | 1766 | 1992 | H3N3 |
| L32024 | A/Seal/Massachusetts/3984/92 | HA (4) | 1766 | 1992 | H3N3 |
| M73776 | A/Mallard/New York/6874/78 | HA (4) | 1765 | 1978 | H3N2 |
| M19057 | A/Swine/Hong Kong/81/78 | HA (4) | 1647 | 1978 | H3N2 |
| D00932 | A/Duck/Hong Kong/231/77 | HA (4) | 1653 | 1977 | H3 |
| D00930 | A/Goose/Hong Kong/10/76 | HA (4) | 1653 | 1976 | H3 |

AACGGAACaATAGTG

| | | | | | |
|---|---|---|---|---|---|
| ISDN64766 | A/Bangkok/1158/2004 | HA (4) | 1023 | 2004 | H3 |
| ISDN64769 | A/Fiji/185/2004 | HA (4) | 1010 | 2004 | H3 |
| ISDN64772 | A/Macau/103/2004 | HA (4) | 1010 | 2004 | H3N2 |
| ISDN64751 | A/Malaysia/1/2004 | HA (4) | 1011 | 2004 | H3N2 |
| ISDN110605 | A/Malaysia/1875/2004 | HA (4) | 1022 | 2004 | H3N2 |
| ISDN64763 | A/Malaysia/452/2004 | HA (4) | 1042 | 2004 | H3 |
| ISDN69020 | A/Malaysia/661/2004 | HA (4) | 1020 | 2004 | H3 |
| AY945273 | A/Nepal/1723/2004 | HA (4) | 1000 | 2004 | H3N2 |
| AY945271 | A/Nepal/1729/2004 | HA (4) | 1000 | 2004 | H3N2 |
| AY619977 | A/Swine/Ontario/42729A/01 | HA (4) | 1701 | 2001 | H3N3 |
| AY619969 | A/Swine/Ontario/K01477/01 | HA (4) | 1701 | 2001 | H3N3 |
| AY633172 | A/mallard/Alberta/199/99 | HA (4) | 1717 | 1999 | H3N6 |
| AY633372 | A/pintail/Alberta/37/99 | HA (4) | 1743 | 1999 | H3N8 |

Table 13 shows results of using a probe that targeted a subset of the 2004 cases. G149A was probed with 18 nucleotides upstream. 10 human isolates were identified and 9 were 2004 isolates from Asia. Most of the isolation dates were available and the earliest 2004 isolate was A/Malaysia/1/2004, isolated on Jan. 12, 2004. However, there was also a 2000 isolate from Sydney. The downstream probe used 11 nucleotides on the 3′ side of the 149 and all human isolates were the 9 from 2004. The same Korean bird isolates, however, also had this polymorphism, as did the swine from Ontario and birds from Alberta. All were H3 but had avian sero-types H3N3, H3N6, and H3N8. Thus, the location of the shared polymorphism was in the middle of a large island of homology between the human isolates and the various avian isolates.

TABLE 14
HA 1016
T1016C
TGTTAAGCAAAACACcCTGAAATTGGCAAC

| | | | | | |
|---|---|---|---|---|---|
| ISDN64771 | A/Macau/214/2004 | HA (4) | 1026 | 2004 | H3N2 |
| ISDN64758 | A/Perth/1/2004 | HA (4) | 1018 | 2004 | H3N2 |
| ISDN110527 | A/Taiwan/1569/2004 | HA (4) | 1011 | 2004 | H3N2 |
| AY862607 | A/chicken/Korea/S6/03 | HA (4) | 1716 | 2003 | H3N2 |
| AY531059 | A/Denmark/18-2/03 | HA (4) | 1701 | 2003 | H3N2 |
| AY862612 | A/dove/Korea/S11/03 | HA (4) | 1415 | 2003 | H3N2 |
| AY862611 | A/duck/Korea/S10/03 | HA (4) | 1410 | 2003 | H3N2 |
| AY862608 | A/duck/Korea/S7/03 | HA (4) | 1412 | 2003 | H3N2 |
| AY862609 | A/duck/Korea/S8/03 | HA (4) | 1410 | 2003 | H3N2 |
| AY862610 | A/duck/Korea/S9/03 | HA (4) | 1412 | 2003 | H3N2 |
| AY963796 | A/Fujian/182/2003 | HA (4) | 1198 | 2003 | H3N2 |
| AY963783 | A/Fujian/219/2003 | HA (4) | 1198 | 2003 | H3N2 |
| AY963784 | A/Fujian/258/2003 | HA (4) | 1198 | 2003 | H3N2 |
| AY963785 | A/Fujian/292/2003 | HA (4) | 1198 | 2003 | H3N2 |
| AY963786 | A/Fujian/325/2003 | HA (4) | 1198 | 2003 | H3N2 |
| AY963787 | A/Fujian/447/2003 | HA (4) | 1198 | 2003 | H3N2 |
| AY695088 | A/Zhejiang/102/03 | HA (4) | 987 | 2003 | H3N2 |
| AY695085 | A/Zhejiang/80/03 | HA (4) | 987 | 2003 | H3N2 |
| AY138516 | A/zhejiang/11/2002 | HA (4) | 987 | 2002 | H3N2 |
| AF315560 | A/Athens/76/98 | HA (4) | 1128 | 1998 | H3N2 |
| AF315562 | A/Greece/103/98 | HA (4) | 1088 | 1998 | H3N2 |
| ISDNSW0001 | A/STOCKHOLM/1/98 | HA (4) | 987 | 1998 | H3N2 |
| AF368439 | A/Finland/460/97 | HA (4) | 984 | 1997 | H3N2 |
| AF368441 | A/Finland/528/97 | HA (4) | 984 | 1997 | H3N2 |
| AF534028 | A/La Plata/12089/97 | HA (4) | 984 | 1997 | H3N2 |
| AF534029 | A/Santa Fe/5456/97 | HA (4) | 984 | 1997 | H3N2 |
| AF008737 | A/Germany/491/96 | HA (4) | 987 | 1996 | H3N2 |
| AY661194 | A/Netherlands/91/96 | HA (4) | 1095 | 1996 | H3N2 |
| AF017270 | A/Vienna/47/96M(MDCK isolate) | HA (4) | 1069 | 1996 | H3N2 |
| AF017271 | A/Vienna/81/96V (Vero isolate) | HA (4) | 1069 | 1996 | H3N2 |
| AY660995 | A/Bilthoven/2668/70 | HA (4) | 1095 | 1970 | H3N2 |
| K03335 | A/England/878/69 | HA (4) | 984 | 1969 | H3N2 |

Table 14 shows the result of using a long 30 nucleotide probe to monitor T1016C which was found in a subset of human isolates and was located in a large island of homology between the Korean avian isolates and human H3N2. The probe identified human European isolates from 1969 to 1998. The polymorphism then was absent from the database until it reappeared in 2003 in humans in China (Fujian and Zhejiang provinces as well as the avian isolates in Korea and one isolate in Denmark). In 2004 it was in three Asian human isolates.

The present findings show that new polymorphism can be used to define small probes to define some regions of genetic transfer, but then the relations identified can be used to develop a fuller algorithm to define key probes by either identifying abrupt changes of hits when used to probe the database or by substituting each of the 4 nucleotides to identify patterns. Most of the key changes are likely to involve transitions at third bases, because most of the changes are synonymous.

This relational database identifies sequences that are on multiple genes at a given time (or on the same gene at distinct locations), or identify patterns that change over time. These critical changes can then be used to both develop vaccines as the virus evolves. Important sequences can be inhibited with interfering RNA (or DNA) that will hybridize and disrupt base paring and assembly.

The developments flow from copy-choice recombination. The present data have shown certain examples where half of a gene matched one source and half the other. However, more common was an aligned sequence pattern where multiple crossovers evolved. This mechanism was used to acquire homologous regions from distant genes including a second location in the same gene, or a short region of homology on another gene.

Recombination was indicated because the recombination process essentially duplicates a short stretch of sequence and puts that stretch on a separate gene. Thus, in the example of the change that created E627K in the PB2 gene in 1997 isolates, the probe found that the same sequences was actually on three of the eight genes of the virus. More often the probe recognized two genes in the same virus, but as the virus evolved over time, these parings changed.

Thus, the recycling of polymorphisms really involves recycling of small regions surrounding these polymorphisms, so the genes are really small stretches strung together in different combinations. These regions limit changes that are possible and because the changes are really recombination events requiring small regions of homology, the pieces move around at different times and on different genes.

Defining genes by small pieces instead of individual nucleotides facilitates design of inhibitors with increased specificity (e.g., RNAi agents), which can change over time. Thus, the relational database requires pairing of sequences on certain gene combinations as well as acknowledging that these relationships change. Thus, a probe that targets HA and NA one year may target PB2 and MP ten years later.

The frequency of these changes is dependent on dual infections with other genomes that allow for change. Human flu appears to change faster than avian (possibly because of the relative size and/or genetic diversity of the human population). Thus, H3N2 in humans evolved more quickly than H3N2 in birds. When the virus crosses species, these different rates can create novel combinations. Thus 2003 ducks and doves in Korea had sequences that were common 20 years earlier in humans. Thus, if a human is infected with a H3N2 bird, it can acquire pieces of the 1983 sequences. That virus is not anticipated to be virulent in those born before 1983, because they have antibodies, but those born after 1983 do not have immunity to the 1983 sequence that suddenly appears.

The above data regarding a human HA sequence from Korean isolates is exemplary of this viral evolution process. This first half of the gene was a contemporary HA H3N2 Fujian sequence. However the 3′ half used sequences from the 1980's which likely moved from humans to a bird in Korea, where it did not change much, but then recombined and reappeared in humans in 2002.

The present findings show several examples of these concepts, all of which use recombination to move small (6-7 nucleotides) to large (half of the gene) sequences from gene to gene.

Thus, flu (and rapidly evolving viruses) use recombination to make changes on a year to year bases, and these changes are accelerated by dual infections with diverse viruses (like influenza A and B) or even Influenza A H5 or H1 and Ebola Spike gene. Such effects are attributable to recombination.

TABLE 15
hth-4

| | | | | |
|---|---|---|---|---|
| At1g72970.1 | 68414.m08439 | glucose-methanol-choline (GMC) oxidored . . . | 42 | 5e−05 |
| At3g18710.1 | 68416.m02376 | U-box domain-containing protein similar . . . | 30 | 0.21 |
| At5g43550.1 | 68418.m05324 | hypothetical protein | 28 | 0.82 |
| At3g50300.1 | 68416.m05501 | transferase family protein similar to a . . . | 28 | 0.82 |
| At1g22710.1 | 68414.m02838 | sucrose transporter/sucrose-proton sy . . . | 28 | 0.82 |
| At3g61680.1 | 68416.m06912 | lipase class 3 family protein contains . . . | 26 | 3.2 |
| At3g50880.1 | 68416.m05571 | HhH-GPD base excision DNA repair family . . . | 26 | 3.2 |
| At1g33350.1 | 68414.m04127 | pentatricopeptide (PPR) repeat-containi . . . | 26 | 3.2 |

hth-8

| | | | | |
|---|---|---|---|---|
| At1g72970.1 | 68414.m08439 | glucose-methanol-choline (GMC) oxidored . . . | 42 | 5e−05 |
| At5g66500.1 | 68418.m08385 | pentatricopeptide (PPR) repeat-containi . . . | 28 | 0.82 |
| At5g46680.1 | 68418.m05752 | pentatricopeptide (PPR) repeat-containi . . . | 28 | 0.82 |
| At4g31310.1 | 68417.m04442 | avirulence-responsive protein-related/ . . . | 28 | 0.82 |
| At1g76830.1 | 68414.m08941 | F-box family protein contains F-box dom . . . | 28 | 0.82 |
| At5g62110.1 | 68418.m07796 | hypothetical protein | 26 | 3.2 |
| At5g25530.1 | 68418.m03038 | DNAJ heat shock protein, putative simla . . . | 26 | 3.2 |
| At4g38280.1 | 68417.m05408 | expressed protein unknown protein F4L23 . . . | 26 | 3.2 |
| At4g14000.1 | 68417.m02165 | expressed protein | 26 | 3.2 |
| At3g48750.1 | 68416.m05324 | cell division control protein 2 homolog . . . | 26 | 3.2 |
| At3g22790.1 | 68416.m02873 | kinase interacting family protein simil . . . | 26 | 3.2 |
| At3g03560.1 | 68416.m00358 | expressed protein | 26 | 3.2 |
| At2g45250.1 | 68415.m05633 | expressed protein | 26 | 3.2 |
| At2g38920.1 | 68415.m04784 | SPX (SYG1/Pho81/XPR1) domain-containing . . . | 26 | 3.2 |
| At2g31540.1 | 68415.m03853 | GDSL-motif lipase/hydrolase family prot . . . | 26 | 3.2 |
| At2g02150.1 | 68415.m00151 | pentatricopeptide (PPR) repeat-containi . . . | 26 | 3.2 |
| At1g43650.1 | 68414.m05011 | integral membrane family protein/nodu . . . | 26 | 3.2 |
| At1g30640.1 | 68414.m03747 | protein kinase, putative contains prote . . . | 26 | 3.2 |
| At1g21060.1 | 68414.m02634 | expressed protein contains Pfam profile . . . | 26 | 3.2 |

hth-10

| | | | | |
|---|---|---|---|---|
| At1g72970.1 | 68414.m08439 | glucose-methanol-choline (GMC) oxidored . . . | 42 | 5e−05 |
| At3g62240.1 | 68416.m06992 | zinc finger (C2H2 type) family protein . . . | 28 | 0.82 |
| At5g58620.1 | 68418.m07346 | zinc finger (CCCH-type) family protein . . . | 26 | 3.2 |
| At5g36170.3 | 68418.m04360 | peptide chain release factor, putative . . . | 26 | 3.2 |
| At5g36170.2 | 68418.m04359 | peptide chain release factor, putative . . . | 26 | 3.2 |
| At5g36170.1 | 68418.m04358 | peptide chain release factor, putative . . . | 26 | 3.2 |
| At5g18090.1 | 68418.m02124 | transcriptional factor B3 family protei . . . | 26 | 3.2 |
| At4g34630.1 | 68417.m04918 | expressed protein | 26 | 3.2 |
| At3g20110.1 | 68416.m02550 | cytochrome P450 family protein similar . . . | 26 | 3.2 |
| At3g14880.1 | 68416.m01881 | DNA-binding protein-related low similar . . . | 26 | 3.2 |
| At2g44710.1 | 68415.m05564 | RNA recognition motif (RRM)-containing . . . | 26 | 3.2 |
| At2g39970.1 | 68415.m04911 | peroxisomal membrane protein (PMP36) id . . . | 26 | 3.2 |
| At1g67340.1 | 68414.m07665 | zinc finger (MYND type) family protein . . . | 26 | 3.2 |

Table 15 (above) shows a list of homology search results (e.g., BLAST results) for three of the HTH mutations (hth-4, hth-8, hth-10). In each case a 21 bp probe was used with the mutated nucleotide at position 11. Listed above are the hits that appeared in the Arabidopsis database (www.arabidopsis.org). The number of 13 or 14 bp exact matches were 7, 18, and 12 respectively. Recombination likely provided a driving force for such genetic transfer in Arabidopsis.

TABLE 16
PB2 polymorphisms A348G, G349A, G351A
CCGACGACAgaTaCAG

| | | | |
|---|---|---|---|
| DQ095761 | A/Bar-headed Goose/Qinghai/12/05 | 2005 | H5N1 |
| DQ095757 | A/Bar-headed Goose/Qinghai/5/05 | 2005 | H5N1 |
| DQ095752 | A/Bar-headed Goose/Qinghai/59/05 | 2005 | H5N1 |
| DQ095755 | A/Bar-headed Goose/Qinghai/60/05 | 2005 | H5N1 |
| DQ095758 | A/Bar-headed Goose/Qinghai/61/05 | 2005 | H5N1 |
| DQ095760 | A/Bar-headed Goose/Qinghai/62/05 | 2005 | H5N1 |
| DQ095762 | A/Bar-headed Goose/Qinghai/65/05 | 2005 | H5N1 |
| DQ095763 | A/Bar-headed Goose/Qinghai/67/05 | 2005 | H5N1 |
| DQ095753 | A/Bar-headed Goose/Qinghai/68/05 | 2005 | H5N1 |
| DQ095759 | A/Bar-headed Goose/Qinghai/75/05 | 2005 | H5N1 |
| DQ100542 | A/black-headed goose/Qinghai/1/2005 | 2005 | H5N1 |
| DQ100543 | A/black-headed goose/Qinghai/2/2005 | 2005 | H5N1 |
| DQ100544 | A/black-headed gull/Qinghai/1/2005 | 2005 | H5N1 |
| DQ095756 | A/Brown-headed Gull/Qinghai/3/05 | 2005 | H5N1 |
| DQ100545 | A/great black-headed gull/Qinghai/1/2005 | 2005 | H5N1 |
| DQ095754 | A/Great Black-headed Gull/Qinghai/2/05 | 2005 | H5N1 |
| AJ293920 | A/Hong Kong/1774/99 | 1999 | H3N2 |

ACAgaTaC

| | | | |
|---|---|---|---|
| DQ095761 | A/Bar-headed Goose/Qinghai/12/05 | 2005 | H5N1 |
| DQ095757 | A/Bar-headed Goose/Qinghai/5/05 | 2005 | H5N1 |
| DQ095752 | A/Bar-headed Goose/Qinghai/59/05 | 2005 | H5N1 |
| DQ095755 | A/Bar-headed Goose/Qinghai/60/05 | 2005 | H5N1 |
| DQ095758 | A/Bar-headed Goose/Qinghai/61/05 | 2005 | H5N1 |
| DQ095760 | A/Bar-headed Goose/Qinghai/62/05 | 2005 | H5N1 |
| DQ095762 | A/Bar-headed Goose/Qinghai/65/05 | 2005 | H5N1 |
| DQ095763 | A/Bar-headed Goose/Qinghai/67/05 | 2005 | H5N1 |
| DQ095753 | A/Bar-headed Goose/Qinghai/68/05 | 2005 | H5N1 |
| DQ095759 | A/Bar-headed Goose/Qinghai/75/05 | 2005 | H5N1 |
| DQ100542 | A/black-headed goose/Qinghai/1/2005 | 2005 | H5N1 |
| DQ100543 | A/black-headed goose/Qinghai/2/2005 | 2005 | H5N1 |
| DQ100544 | A/black-headed gull/Qinghai/1/2005 | 2005 | H5N1 |
| DQ095756 | A/Brown-headed Gull/Qinghai/3/05 | 2005 | H5N1 |
| DQ100545 | A/great black-headed gull/Qinghai/1/2005 | 2005 | H5N1 |
| DQ095754 | A/Great Black-headed Gull/Qinghai/2/05 | 2005 | H5N1 |
| AJ306841 | A/swine/Cotes d'Armor/800/00 | 2000 | H1N2 |
| AJ306846 | A/swine/Italy/1081/00 | 2000 | H1N2 |
| AJ293920 | A/Hong Kong/1774/99 | 1999 | H3N2 |
| AJ306855 | A/swine/Cotes d'Armor/1482/99 | 1999 | H1N1 |
| AJ306848 | A/swine/Cotes d'Armor/1488/99 | 1999 | H1N1 |
| AJ306842 | A/swine/Ille et Vilaine/1455/99 | 1999 | H1N1 |
| AJ306853 | A/swine/Italy/1657/99 | 1999 | H1N1 |
| AJ306843 | A/swine/Italy/2064/99 | 1999 | H1N2 |
| AJ306854 | A/swine/Cotes d'Armor/2433/98 | 1998 | H1N2 |
| AJ306849 | A/swine/Italy/1521/98 | 1998 | H1N2 |
| AJ311459 | A/Swine/Italy/1523/98 | 1998 | H3N2 |
| AJ306845 | A/swine/Cotes d'Armor/790/97 | 1997 | H1N2 |

Table 16 shows the result of probing three polymorphisms (A346G, G347A, G349A) with a 16 nucleotide sequence. The sequence identified all 16 isolates from Qinghai Lake as well as one additional isolate, A/Hong Kong/1774/99. This was an H3N2 isolate from a child in Hong Kong in 1999. The isolate was notable because it was closely related to European swine isolates, but the child had not traveled outside of Hong Kong. A smaller probe of 8 nucleotides was used to identify additional isolates that had all three polymorphisms, and 11 additional sequences were identified. These 11 sequences were isolated from European swine, with H1N1, H1N2, and H3N2 serotypes, and this finding confirmed earlier reports that the Hong Kong isolate was similar to European swine. These isolates could be identified with the small 8 nucleotide probe, which demonstrated that this small region of sequence in PB2 moved from European swine, then to Hong Kong, and to Qinghai lake. These observations also identified a small mammalian region within a PB2 sequence that contained an H5N1 background sequence.

TABLE 17
HA C631T
GGGGGATCCAt

| | | | |
|---|---|---|---|
| DQ007623 | A/Anas platyrhynchos/Chany Lake/9/03 | 2003 | H5N3 |
| AF202256 | A/teal/Taiwan/WB2-37-2TPFE2/98 | 1998 | H7N4 |
| AF310989 | A/Red Kont/Delaware/254/94 | 1994 | H8N4 |
| AF202239 | A/chicken/Ireland/1733/89 | 1989 | H7N7 |
| M24727 | A/Equine/Kentucky/2/86 | 1986 | H3N8 |
| M24725 | A/Equine/Santiago/1/85 | 1985 | H3N8 |
| AY383755 | A/equine/Santiago/85 | 1985 | H3N8 |
| Y14055 | A/equi 2/Aby/84 HA | 1984 | H3N8 |
| AF310988 | A/Mallard Duck/Alberta/357/84 | 1984 | H8N4 |
| U58195 | A/Equine/Kentucky/1/81 | 1981 | H3N8 |
| M24724 | A/Equine/Romania/80 | 1980 | H3N8 |
| D30677 | A/Equine/NewMarket/D64/79 | 1979 | H3N8 |
| AF310987 | A/Pintail Duck/Alberta/114/79 | 1979 | H8N4 |
| M24723 | A/Equine/Fontainebleau/76 | 1976 | H3N8 |
| M73773 | A/Equine/France/1/76 | 1976 | H3N8 |
| M24722 | A/Equine/Newmarket/76 | 1976 | H3N8 |
| M24721 | A/Equine/Algiers/72 | 1972 | H3N8 |
| M24720 | A/Equine/Tokyo/71 | 1971 | H3N8 |
| D90304 | A/turkey/Ontario/6118/68 | 1968 | H8N4 |
| M24719 | A/Equine/Miami/1/63 | 1963 | H3N8 |
| M29257 | A/Equine/Miami/63 | 1963 | H3N8 |
| M24718 | A/Equine/Uruguay/1/63 | 1963 | H3N8 |
| X07826 | A/Chicken/Scotland/59 | 1959 | H5N1 |
| X07869 | A/chicken/Scotland/59 | 1959 | H5N1 |

The short probe shown in Table 17 (above) corresponds to the region upstream of polymorphism C631T, and was used to identify genes which share this region. The probe identified serotypes that ultimately were found in human infections, but it identified these serotypes years before the sero-type developed the ability to cause human disease. Thus, this region predicted human sero-types before they acquired the ability to infect humans and it identified a region in the viral evolution. The region was present in the first H5N1 isolate from 1959. 38 years later, the first infection of H5N1 in a person was reported. The probe identified H3 in 1963. The first H3N2 infection in humans was in the 1968 pandemic. H7 was identified in a 1989 isolate and in a human infection in 2003 as H7N7. The probe also identified several H8 isolates. The earliest was 1979. There have not yet been any reported human cases involving H8, but these results anticipate that recombination involving H8 will result in a future human pandemic.

TABLE 18
HA A640G
gAAGCTCTAT

| | | | |
|---|---|---|---|
| DQ007623 | A/Anas platyrhynchos/Chany Lake/9/03 | 2003 | H5N3 |
| AY531029 | A/Mallard/64650/03 | 2003 | H5N7 |
| AB099427 | C/Hiroshima/247/2000 | 2000 | |
| AB099428 | C/Hiroshima/248/2000 | 2000 | |
| AB099425 | G/Hiroshima/249/2000 | 2000 | |
| AB099417 | C/Miyagi/3/2000 | 2000 | |
| AB099418 | C/Miyagi/4/2000 | 2000 | |
| AB099420 | C/Saitama/1/2000 | 2000 | |
| AB099412 | C/Yamagata/2/2000 | 2000 | |
| AB099414 | C/Yamagata/6/2000 | 2000 | |
| AB099415 | C/Yamagata/8/2000 | 2000 | |
| AB099411 | C/Yamagata/9/2000 | 2000 | |
| AY684894 | A/mallard/Netherlands/3/99 | 1999 | H5N2 |
| AB099424 | C/Hiroshima/252/99 | 1999 | |
| AB099423 | C/Hiroshima/290/99 | 1999 | |
| AB086668 | C/Miyagi/1/99 | 1999 | |
| AB086669 | C/Miyagi/3/99 | 1999 | |
| AB099410 | C/Yamagata/2/99 | 1999 | |
| AB086666 | C/Miyagi/2/98 | 1998 | |
| AB086667 | C/Miyagi/4/98 | 1998 | |
| AB064403 | C/Yamagata/13/98 | 1998 | |
| AB064401 | C/Yamagata/2/98 | 1998 | |
| AB064402 | C/Yamagata/6/98 | 1998 | |
| AB086665 | C/Miyagi/1/97 | 1997 | |
| AB086662 | C/Miyagi/2/96 | 1996 | |
| AB086663 | C/Miyagi/6/96 | 1996 | |
| AB064400 | C/Yamagata/20/96 | 1996 | |
| AB064397 | C/Yamagata/3/96 | 1996 | |
| AB064398 | C/Yamagata/8/96 | 1996 | |
| AB064399 | C/Yamagata/9/96 | 1996 | |
| D87392 | C/Miyagi/2/92 | 1992 | |
| D87393 | C/Miyagi/3/91 | 1991 | |
| AB086660 | C/Miyagi/7/91 | 1991 | |
| D87394 | C/Miyagi/9/91 | 1991 | |
| AB086658 | C/Miyagi/1/90 | 1990 | |
| D49361 | C/Yamagata/10/89 | 1989 | |
| D88428 | C/Yamagata/4/89 | 1989 | |
| D63473 | C/Yamagata/3/88 | 1988 | |
| D63503 | C/Yamagata/4/88 | 1988 | |
| D28967 | C/Yamagata/7/88 | 1988 | |
| D28968 | C/Yamagata/8/88 | 1988 | |
| D28969 | C/Yamagata/9/88 | 1988 | |
| AB086657 | C/Yamagata/1/86 | 1986 | |
| D30697 | C/Nara/2/85 | 1985 | |
| M11642 | C/England/892/83 | 1983 | |
| M11645 | C/pig/Beijing/439/82 | 1982 | |
| M11643 | C/pig/Beijing/10/81 | 1981 | |
| M11644 | C/pig/Beijing/115/81 | 1981 | |
| M11641 | C/Yamagata/10/81 | 1981 | |
| D28971 | C/Yamagata/26/81 | 1981 | |
| D63472 | C/Kyoto/79 | 1979 | |
| AB086656 | C/Shizuoka/79 | 1979 | |
| K01689 | C/California/78 | 1978 | |
| AB035362 | C/New Jersey/1/76 | 1976 | |
| D63468 | C/Sapporo/71 | 1971 | |
| M11638 | C/Ann Arbor/1/50 | 1950 | |

Table 18 (above) shows the result of using the 9 nucleotides downstream from A640G to track the sequence found in HA of an H5N3 migratory bird isolate from Chany Lake. Two other H5s were identified from Europe. However, the sequence was found in the analogous gene, HEF, in influenza C. The sequence could be found as early as 1950 and was present worldwide in humans as well as swine isolates in China. Thus, Influenza C can also act as a donor region for Influenza A.

TABLE 19A

Common and Closely Related Sequences Between HA in H5 Influenza
and env in Ebola

| | | | |
|---|---|---|---|
| Probe: | 1 | GTTCTTGGTCCAATCATG | 18 |
| 32165589 | 359 | .................. | 376 |
| 32165587 | 359 | .................. | 376 |
| 32165587 | 955 | .......... | 946 |
| 32165577 | 359 | .................. | 376 |
| 32165575 | 359 | .................. | 376 |
| 32165571 | 419 | .................. | 436 |
| 50952819 | 277 | .................. | 294 |
| 23630482 | 7891 | .................. | 7874 |
| 23630482 | 11643 | .......... | 11634 |
| 70955443 | 407 | .................. | 424 |
| 46981858 | 407 | .................. | 424 |
| 55233227 | 435 | .................. | 452 |
| 54126496 | 435 | .................. | 452 |
| 2522270 | 7894 | .................. | 7877 |
| 2522270 | 11643 | .......... | 11634 |
| 21702647 | 7891 | .................. | 7874 |
| 21702647 | 11644 | .......... | 11635 |
| 1141778 | 1992 | .................. | 1975 |
| 1695251 | 1994 | .................. | 1977 |
| 1041204 | 1994 | .................. | 1977 |
| 1753170 | 1994 | .................. | 1977 |

| | | |
|---|---|---|
| gi\|32165589\|gb\|AY296084.1\| Influenza A virus (A/ruddy turnsto . . . | 36.2 | 0.012 |
| gi\|32165587\|gb\|AY296083.1\| Influenza A virus (A/turkey/CA/D02 . . . | 36.2 | 0.012 |
| gi\|32165577\|gb\|AY296078.1\| Influenza A virus (A/unknown/NY/11 . . . | 36.2 | 0.012 |
| gi\|32165575\|gb\|AY296077.1\| Influenza A virus (A/duck/NJ/11722 . . . | 36.2 | 0.012 |
| gi\|32165571\|gb\|AY296075.1\| Influenza A virus (A/unknown/NY/98 . . . | 36.2 | 0.012 |
| gi\|50952819\|gb\|AY623430.1\| Influenza A virus (A/chicken/Yicha . . . | 36.2 | 0.012 |
| gi\|23630482\|gb\|AY142960.1\| Zaire Ebola virus strain Mayinga subt | 36.2 | 0.012 |
| gi\|70955443\|gb\|DQ095628.1\| Influenza A virus (A/Goose/Shantou . . . | 36.2 | 0.012 |
| gi\|46981858\|gb\|AY531029.1\| Influenza A virus (A/Mallard/64650 . . . | 36.2 | 0.012 |
| gi\|55233227\|gb\|AY770079.1\| Influenza A virus (A/chicken/Hubei . . . | 36.2 | 0.012 |
| gi\|54126496\|gb\|AY747609.1\| Influenza A virus (A/swine/Fujian/ . . . | 36.2 | 0.012 |
| gi\|2522270\|gb\|L11365.1\|EBORNA Zaire Ebola virus nucleoprotein . . . | 36.2 | 0.012 |
| gi\|21702647\|gb\|AF499101.1\| Zaire Ebola virus strain Mayinga, com | 36.2 | 0.012 |
| gi\|1141778\|gb\|U31033.1\|EVU31033 Zaire Ebola virus envelope gl . . . | 36.2 | 0.012 |
| gi\|1695251\|gb\|U28077.1\|EVU28077 Zaire Ebola virus strain Zair . . . | 36.2 | 0.012 |
| gi\|1041204\|gb\|U23187.1\|EVU23187 Zaire Ebola virus Mayinga str . . . | 36.2 | 0.012 |
| gi\|1753170\|gb\|U81161.1\|EVU81161 Zaire Ebola virus virion spik . . . | 36.2 | 0.012 |
| gi\|10141003\|gb\|AF086833.2\| Zaire Ebola virus strain Mayinga, com | 36.2 | 0.012 |
| gi\|l1761745\|gb\|AF272001.1\| Zaire Ebola virus strain Mayinga, com | 36.2 | 0.012 |
| gi\|57208072\|emb\|AJ621811.1\| Influenza A virus (A/duck/Primori . . . | 36.2 | 0.012 |

TABLE 19B
HA Region in Migratory Bird Sequence Corresponding to Homology
Region Between H5 and Ebola

| Query | 1 GTTCTTGGTCAGATCATG 18 |
|---|---|
| 71068504 | 331 . . . 348 |
| 71025275 | 331 . . . 348 |
| 71025273 | 331 . . . 348 |
| 71025271 | 331 . . . 348 |
| 70955436 | 407 . . . 424 |
| 70955434 | 407 . . . 424 |
| 70955433 | 407 . . . 424 |
| 70955431 | 407 . . . 424 |
| 70955429 | 407 . . . 424 |
| 70955428 | 407 . . . 424 |
| 70955427 | 407 . . . 424 |
| 70955426 | 407 . . . 424 |
| 70955425 | 407 . . . 424 |
| 70955423 | 371 . . . 388 |
| 70955422 | 407 . . . 424 |
| 70955421 | 407 . . . 424 |
| 58805 | 3309 . . . 3296 |
| 70955447 | 404 . . . C . . . 421 |
| 70955445 | 404 . . . C . . . 421 |
| 70955444 | 407 . . . C . . . 424 |
| 70955442 | 407 . . . C . . . 424 |
| 70955440 | 407 . . . C . . . 424 |
| 57208068 | 392 . . . C . . . 409 |
| 19171520 | 9391 . . . 9378 |
| 19171520 | 6001 . . . 5992 |
| 6138913 | 1670 . . . 1657 |
| 58531156 | 407 . . . C . . . 424 |
| 58531138 | 407 . . . C . . . 424 |
| 58531120 | 407 . . . C . . . 424 |
| 58531088 | 407 . . . C . . . 424 |
| 67972965 | 61164 . . . 61177 |
| 50296128 | 380 . . . C . . . 397 |
| 50296126 | 380 . . . C . . . 397 |
| 50296124 | 407 . . . C . . . 424 |
| 50296122 | 407 . . . C . . . 424 |
| 50296120 | 407 . . . C . . . 424 |
| 50296116 | 380 . . . C . . . 397 |
| 50296114 | 380 . . . C . . . 397 |
| 50296112 | 380 . . . C . . . 397 |
| 50296110 | 407 . . . C . . . 424 |
| 50296100 | 407 . . . C . . . 424 |
| 50296034 | 407 . . . C . . . 424 |
| 50296032 | 407 . . . C . . . 424 |
| 50296030 | 407 . . . C . . . 424 |
| 50296028 | 407 . . . C . . . 424 |
| 50296026 | 407 . . . C . . . 424 |
| 50296024 | 407 . . . C . . . 424 |
| 53913362 | 259 . . . 246 |
| 56548877 | 407 . . . C . . . 424 |
| 56548875 | 407 . . . C . . . 424 |
| 58374179 | 435 . . . C . . . 452 |
| 57916028 | 435 . . . C . . . 452 |
| 47716772 | 435 . . . C . . . 452 |
| 3056875 | 1535 . . . 1522 |
| 41207462 | 435 . . . C . . . 452 |
| 47156294 | 407 . . . C . . . 424 |
| 335150 | 2350 . . . 2363 |
| 323880 | 92 . . . 79 |
| 37935899 | 6984 . . . 6996 |
| 37956854 | 22709 . . . 22697 |
| 37956799 | 22719 . . . 22707 |
| 37956749 | 21292 . . . 21280 |
| 37956696 | 22716 . . . 22704 |
| 37956638 | 23561 . . . 23549 |
| 26284361 | 12085 . . . 12073 |
| 33347893 | 88344 . . . 88356 |
| 33347893 | 12486 . . . 12495 |
| 33347893 | 87598 . . . 87589 |
| 56160984 | 640916 . . . A . . . 640903 |
| 56160984 | 3073013 . . . A . . . 3073026 |
| 56160984 | 3382849 . . . A . . . 3382836 |
| 56160984 | 121560 . . . 121548 |
| 56160984 | 1904464 . . . 1904476 |
| 56160984 | 117692 . . . 117682 |
| 56160984 | 295248 . . . 295258 |
| 56160984 | 915450 . . . 915440 |
| 56160984 | 939400 . . . 939390 |
| 56160984 | 1571947 . . . 1571957 |
| 56160984 | 1653825 . . . 1653815 |
| 56160984 | 1719470 . . . 1719460 |
| 56160984 | 2174905 . . . 2174895 |
| 56160984 | 3168320 . . . 3168330 |
| 56160984 | 3451891 . . . 3451901 |
| 56160984 | 4033934 . . . 4033944 |
| 56160984 | 715 . . . 706 |
| 56160984 | 131017 . . . 131008 |
| 56160984 | 146601 . . . 146610 |
| 56160984 | 153011 . . . 153002 |
| 56160984 | 225257 . . . 225248 |
| 56160984 | 267266 . . . 267257 |
| 56160984 | 314428 . . . 314419 |

| Sequences producing significant alignments: | (Bits) | Value |
|---|---|---|
| gi\|71068504\|gb\|DQ100557.2\|Influenza A virus | 36.2 | 0.012 |
| (A/great black-h . . . | | |
| gi\|71025275\|gb\|DQ100556.1\|Influenza A virus | 36.2 | 0.012 |
| (A/black-headed . . . | | |
| gi\|71025273\|gb\|DQ100555.1\|Influenza A virus | 36.2 | 0.012 |
| (A/black-headed . . . | | |
| gi\|71025271\|gb\|DQ100554.1\|Influenza A virus | 36.2 | 0.012 |
| (A/black-headed . . . | | |
| gi\|70955436\|gb\|DQ095623.1\|Influenza A virus | 36.2 | 0.012 |
| (A/Bar-headed Go . . . | | |
| . | | |
| . | | |
| . | | |
| gi\|56160984\|gb\|CP000002.2\|Bacillus licheniformis | 26.3 | 11 |
| ATCC 14580, co | | |
| . | | |
| . | | |
| . | | |
| gi\|22770789\|gb\|AF536539.1\|Foot-and-mouth disease | 24.3 | 45 |
| virus SAT 3 . . . | | |
| gi\|22770787\|gb\|AF536538.1\|Foot-and-mouth disease | 24.3 | 45 |
| virus SAT 2 . . . | | |
| gi\|22770785\|gb\|AF536537.1\|Foot-and-mouth disease | 24.3 | 45 |
| virus SAT 1 . . . | | |

Table 19A (above) shows the top 20 hits from the 1182 hits obtained via BLAST searching using the 18 nucleotide probe. The probe was an exact match between many H5 influenza isolates as well as Ebola isolates. A large number of West Nile isolates were also recognized with this probe, indicating genetic transfer (recombination) between Ebola and influenza and West Nile and influenza.

Table 19B (above) shows representative hits culled from 1395 hits for an 18-mer probe corresponding to a region of homology between HA from H5 influenza and Ebola env. This probe represented the bar migratory bird sequences from Qinghai Lake. Only a small subset of H5s were identified, but the two nucleotide changes matched the sequence in many foot-and-mouth isolates. Included in the matches were 55 regions of homology in industrial Bacillus licheniformis ATCC 14580. Thus, genetic transfer events have occurred via recombination between foot-and-mouth isolates and influenza, as well as between industrial Bacillus licheniformis ATCC 14580 and influenza.

Because viruses evolve via recombination, unique polymorphisms can be used to trace viral sequences back to parental viruses. This approach was used to find the origins of polymorphisms that were in H5N1 sequences from Vietnam and Thailand but essentially absent in other H5N1 sequences.

This general approach was refined to perform three sets of searches with the polymorphism in question on the right, left, and in the middle. This approach identified short stretches of homology (the search was for exact matches) and the process was used to see how genes assembled, see how new serotypes emerged, and find parental sequences. Since the sequences probed evolved over a series of recombinations involving different parents at different times, there were only a few polymorphisms at various locations.

The approach was used to analyze sequences from Qinghai Lake. These sequences are important because normally migratory birds are not killed by H5N1 and it was thought that they don't transmit. However, an existing sequence was used herein to determine the origin of the polymorphisms, which revealed H5 does transmit from migratory birds. Such transmission creates a major problem as the birds leave Qinghai lake to travel throughout Asia, Europe, and probably North America.

The existing sequence most closely related to the Qinghai sequences was a 2003 sequence from Shantou Province. The present analysis of origins showed that migratory birds infected chickens in Shantou, which is why there was a match.

Polymorphisms in the Shantou sequences were used to probe the database at Los Alamos. Several of the polymorphisms were found in a wide assortment of H5 serotypes in isolates from migratory birds dating back to the 1970's. These data revealed these migratory bird sequences were in Shantou in 2003 and revealed that birds from Qinghai Lake and other locations brought the sequences to Shantou, which is why there were matches.

These findings demonstrated the power of tracing individual polymorphisms and supported recombination happening year after year to integrate new polymorphisms via migratory birds. It also manifested the need for an extensive flu sequence/polymorphism database. Such a database can aid in vaccine technology.

The other discovery that was evidenced in these searches was recombination between more distant species. This analysis showed that influenza B provides a few of the polymorphisms found in Vietnam and Thailand. There are many types of homologous recombination: same region—same gene; different region—same gene; different gene; other family member (influenza A and B); more distant relationship (H1, H5, ebola spike—opposite strand); and there is also non-homologous recombination.

The present findings are important for public health and call for major changes in medical practice (such as not allowing sick people to sit in close quarters in waiting rooms). New viruses in people are being formed all of the time via copy-choice recombination and viral gene pools are getting increasingly diverse and unstable as a result. Many viruses have short regions of homology, and dual infections are leading to much more recombination.

The analyses of the present invention can be used to enhance predictive powers. The influenza field has assumed that genetic information flow moves from domestic birds to migratory birds, when in fact the flow is the opposite. Thus, the migratory strains are more complex with regard to serotypes with polymorphisms being widely dispersed, while the domestic poultry outbreaks are traced to more common sources.

There is also information flow from migratory birds to human sequences, which is why bird sequences of at least one of the above tables are listed earlier than the human sequences (the polymorphisms moved from migratory birds to mammals and now back to pandemic virus). However, the earlier isolates were on a migratory bird background, then human causing drifting, and down pandemic strains, which were avian but now expanding host range with human sequences.

Thus, patterns as detected by the methods of the present invention can help determine sources and information flow.

The analyses of the present invention can be readily updated upon deposit of additional sequences into viral databases (comprehensively including those presently analyzed and all other sources of viral sequence). Performance of the analyses of the present invention in an iterative and updated manner is therefore envisioned and within the scope of the present invention.

The analyses of the present invention can also be performed on any one or more of the viral strains listed in any of the Tables.

### Example 4

### Prediction of Specific Emerging Progeny Strain Sequences

The preceding examples document transmission of polymorphic sequences via copy-choice recombination. The preceding examples also show that knowledge of the role of copy-choice recombination in transmission of polymorphic sequences can be efficiently used to predict progeny sequences that are likely to result from mixing of two parental sequences in a single host animal. In the present example, such an approach was successfully applied to predict the time, nature and geographic location of emergence for a specific progeny strain of H5N1 influenza. To make this prediction, an initial search was performed to identify parental viral strain sequences comprising specific polymorphisms of molecular, clinical or pathological importance, with elevated interest in those that were likely to be transferred to a second parental viral strain via copy-choice recombination. The recently-described S227N polymorphism discovered in two H5N1 isolates from Hong Kong in 2003 (ex Fujian Province) had been identified as a polymorphism of molecular and clinical importance, as the two H5N1 isolates harboring the S227N polymorphism had been documented in one study to increase binding efficiencies for human receptors in the upper respiratory tract. Moreover, the S227N polymorphism was also shown to enhance the immunogenicity of H5 (Hoffmann et al., Proc. Natl. Acad. Sci. 102: 12915-20). However, the S227N polymorphism was rare in H5N1 (a single additional isolate from Vietnam was identified in 2005, but did not register as a hit in the present search because the sequence had not been deposited at Los Alamos). Following identification of the S227N polymorphism as a candidate for gene transfer, a 10 nucleotide probe sequence was used to search for viral strains currently possessing the polymorphism. The hits generated by this search are shown in Table 20A below.

TABLE 20A
Search Results for S227N Viral Strains

| | | | |
|---|---|---|---|
| AY575869 | A/Hong Kong/212/03 | 2003 | H5N1 |
| AB212054 | A/Hong Kong/213/03 | 2003 | H5N1 |
| ISDN38262 | A/Hong Kong/213/2003 | 2003 | H5N1 |
| AY575870 | A/Hong Kong/213/2003 | 2003 | H5N1 |
| AY738456 | A/ostrich/Eshkol/1436/03 | 2003 | H9N2 |
| DQ104453 | A/turkey/Avigdor/1209/03 | 2003 | H9N2 |
| DQ104454 | A/turkey/Avigdor/1215/03 | 2003 | H9N2 |
| DQ104458 | A/turkey/Brosh/1276/03 | 2003 | H9N2 |
| DQ104455 | A/turkey/Kfar Warburg/1224/03 | 2003 | H9N2 |
| DQ104464 | A/chicken/Kfar Monash/636/02 | 2002 | H9N2 |
| DQ104450 | A/turkey/Avichail/1075/02 | 2002 | H9N2 |
| DQ104473 | A/turkey/Beit HaLevi/1009/02 | 2002 | H9N2 |
| DQ104451 | A/turkey/Ein Tzurim/1172/02 | 2002 | H9N2 |
| DQ104462 | A/turkey/Givat Haim/622/02 | 2002 | H9N2 |
| DQ104471 | A/turkey/Givat Haim/868/02 | 2002 | H9N2 |
| AY548507 | A/turkey/Givat Haim/965/02 | 2002 | H9N2 |
| AY738452 | A/turkey/Givat Haim/965/02 | 2002 | H9 |
| DQ104459 | A/turkey/Kfar Vitkin/615/02 | 2002 | H9 |
| AY548510 | A/turkey/Kfar Vitkin/615/02 | 2002 | H9N2 |
| DQ104460 | A/turkey/Kfar Vitkin/616/02 | 2002 | H9 |
| AY548511 | A/turkey/Kfar Vitkin/616/02 | 2002 | H9N2 |
| AY548512 | A/turkey/Mishmar Hasharon/619/02 | 2002 | H9N2 |
| DQ104461 | A/turkey/Mishmar Hasharon/619/02 | 2002 | H9 |
| DQ104449 | A/turkey/Naharia/1013/02 | 2002 | H9N2 |
| DQ104452 | A/turkey/Sapir/1199/02 | 2002 | H9N2 |
| DQ104463 | A/turkey/Yedidia/625/02 | 2002 | H9 |
| AY548513 | A/turkey/Yedidia/625/02 | 2002 | H9N2 |
| DQ104448 | A/turkey/Yedidia/911/02 | 2002 | H9N2 |
| AY548514 | A/chicken/Tel Adashim/786/01 | 2001 | H9N2 |
| AY738454 | A/chicken/Tel Adashim/786/01 | 2001 | H9 |
| DQ104465 | A/chicken/Tel Adashim/809/01 | 2001 | H9 |
| AY548515 | A/chicken/Tel Adashim/809/01 | 2001 | H9N2 |
| AY548500 | A/chicken/Tel Adashim/811/01 | 2001 | H9N2 |
| DQ104467 | A/chicken/Tel Adashim/811/01 | 2001 | H9 |
| AY548501 | A/chicken/Tel Adashim/812/01 | 2001 | H9N2 |
| DQ104468 | A/chicken/Tel Adashim/812/01 | 2001 | H9 |
| DQ104469 | A/goose/Tel Adashim/829/01 | 2001 | H9N2 |
| DQ104470 | A/goose/Tel Adashim/830/01 | 2001 | H9N2 |
| AY548499 | A/turkey/Givat Haim/810/01 | 2001 | H9N2 |
| DQ104466 | A/turkey/Givat Haim/810/01 | 2001 | H9 |
| DQ104472 | A/chicken/Maale HaHamisha/90658/00 | 2000 | H9N2 |
| AY738451 | A/turkey/Neve Ilan/90710/00 | 2000 | H9 |
| AY548502 | A/turkey/Neve Ilan/90710/00 | 2000 | H9N2 |
| AF218108 | A/Peking Duck/Malaysia/F20/1/98 | 1998 | H9N2 |
| AF218105 | A/Peking Duck/Singapore/F91-5/9/97 | 1997 | H9N2 |
| AY206675 | A/quail/Hong Kong/A28945/88 | 1988 | H9N2 |

Strikingly, all of the most recent hits for the S227N polymorphism were observed in H9N2 viral strain sequences identified in isolates in the Middle East (whereas recent H9N2 isolates from Asia did not possess the donor (S227N) sequence).

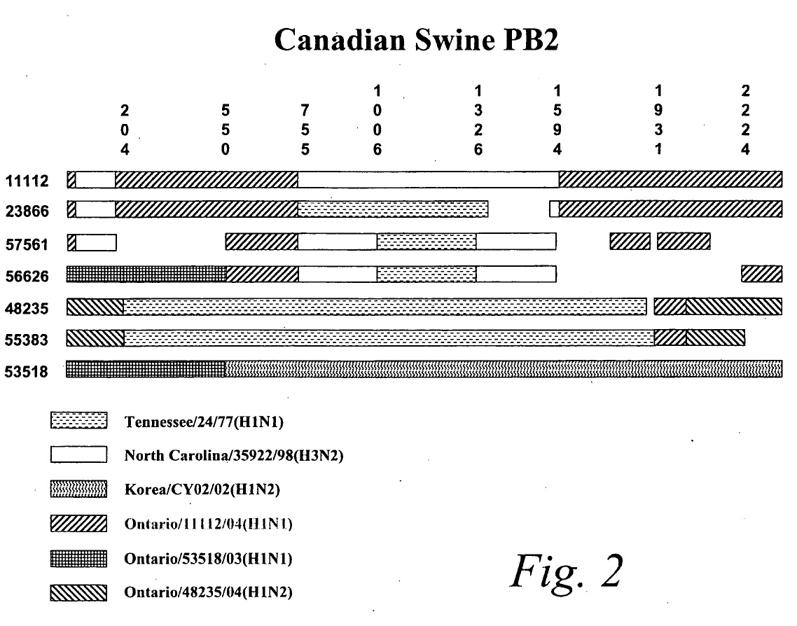

Thus, this approach, when considered in view of wild bird migration routes (refer to FIG. 2), not only predicted that the S227N polymorphism would likely be found in H5N1 progeny strains, but also predicted the time and location of emergence of such strains. Specifically, wild bird H5N1 HA sequences were predicted as the recipient of the S227N polymorphism from H9N2 strains in the Middle East, as wild bird H5N1 strains were anticipated to migrate into the Middle East in the autumn of 2005. Because H5N1 had not been previously reported to have spread to the Middle East, this wild bird migration represented a new opportunity for H5N1 to infect H9N2-infected poultry (H9N2 infections are endemic to poultry in the Middle East). The dual infection would allow for recombination and the acquisition of S227N by H5N1, which, in turn, would lead to human cases because of the increased affinity of the new receptor binding domain for human cell surface receptor(s).

The present predictive method targeted a specific polymorphism and searched for donor sequences that could recombine with one parent viral strain sequence (wild bird H5N1 sequence) to allow that polymorphism to be acquired. One advantage of this approach is found in the ability of the result of such a search to be analyzed in terms of the likelihood of a future event. Thus, the fact that the donor sequences were H9N2 in the Middle East (a geographic region where H5N1 had not been previously found) and not in H9N2 in Asia, where H5N1 had previously spread, made the donor sequences particularly relevant. The fact that the H5N1 parental viral strain could be moving into the geographic area of the Middle East in the autumn of 2005 also allowed for prediction of a time and geographic location for the gene transfer event to occur; and because the polymorphism modulated the receptor binding domain of HA, the change was predicted to increase the efficiency of infections in humans (an effect that would be further enhanced by the acquisition of PB2 E627K, which had occurred in wild bird H5N1 parental viral strain isolates a few months earlier (May, 2005) at Qinghai Lake in China).

The preceding approach was also applied to other polymorphisms present in HA receptor binding domains that were shown to increase affinity for human receptors. For example, spread of the G228S polymorphism, which switches receptor binding domain affinity from avian to human, was examined. Prediction of the geographic spread of the G228S polymorphism was similar to that of the S227N documented above. As for S227N strains, polymorphism G228S-containing (donor) parental viral strain sequences were localized to a region (in the case of G228S, Europe) where H5N1 viral strains (in wild birds from Asia) had not yet been documented to have circulated. However, unlike S227N (observed in H9N2 strains), the G228S polymorphism was observed in H1N1 strains. H1N1 is a viral strain endemic to Europe. H5N1 had not yet been documented in Europe, yet in Asia, H5N1 had been observed to infect swine, generally asymptomatically. Migratory birds infected with H5N1 were predicted to spread to Europe in the Spring of 2006. Accordingly, the prediction was made that a dual infection (of a swine, migratory bird, or even a distinct host animal, e.g., human, in Europe) by H5N1 (migratory bird parental strain) and H1N1 (G228S-containing strain endemic to European swine) would result in the H5N1 viral strain acquiring the G228S polymorphism from the H1N1 strain by a gene transfer event.

Use of the probe sequence “aGcAGGATGGA” produced the following hits, all of which (with the exception of the one hit, AJ517815, not currently shown) were isolated from European swine. (Although the AJ517815 sequence was isolated from a human, it is essentially a European swine sequence, demonstrating that there is the rare chance of recombination between H1N1 in a human infected with a swine sequence and then infected with H5N1—this event would probably be much less likely than H1N1 in swine, bit it does represent another opportunity for such an event to occur.)

TABLE 20B
Probe Sequence aGcAGGATGGA Used to Identify G228S Strains
G228S
G746A A748C
Probe: aGcAGGATGGA

| | | | |
|---|---|---|---|
| AJ517820 | A/swine/Cotes d'Armor/736/2001 | 2001 | H1N1 |
| AJ517818 | A/swine/Italy/10951/2001 | 2001 | H1N1 |
| AJ517817 | A/swine/Italy/13260/2001 | 2001 | H1N1 |
| AJ412708 | A/swine/Cotes d'Armor/1121/00 | 2000 | H1N1 |
| AJ344004 | A/swine/Cotes d'Armor/918/00 | 2000 | H1N1 |
| AJ517819 | A/swine/Italy/3088/00 | 2000 | H1N1 |
| AJ344018 | A/swine/Italy/3364/00 | 2000 | H1N1 |
| AJ344021 | A/swine/Cotes d'Armor/1455/99 | 1999 | H1N1 |
| AJ412711 | A/swine/Cotes d'Armor/1482/99 | 1999 | H1N1 |
| AJ412712 | A/swine/Cotes d'Armor/1488/99 | 1999 | H1N1 |
| AJ344002 | A/swine/Cotes d'Armor/1515/99 | 1999 | H1N1 |
| AJ344013 | A/swine/Italy/2064/99 | 1999 | H1N2 |
| AY590824 | A/swine/Belgium/1/98 | 1998 | H1N1 |
| AJ344016 | A/swine/Italy/1511/98 | 1998 | H1N1 |
| AJ344020 | A/swine/Italy/1589/98 | 1998 | H1N1 |
| AJ344019 | A/swine/Italy/1498-2/97 | 1997 | H1N1 |
| AJ344017 | A/swine/Italy/1509-6/97 | 1997 | H1N1 |
| AJ344008 | A/swine/Italy/1456-1/96 | 1996 | H1N1 |
| AF320064 | A/Swine/Netherlands/609/96 | 1996 | H1N1 |
| AJ344009 | A/swine/Italy/1390-5/95 | 1995 | H1N1 |
| AJ344011 | A/swine/Italy/France/2111/95 | 1995 | H1N1 |
| AF320059 | A/Swine/France/525/94 | 1994 | H1N1 |
| AF320063 | A/Swine/Netherlands/1743/93 | 1993 | H1N1 |
| AF320066 | A/Swine/Netherlands/477/93 | 1993 | H1N1 |
| U72669 | A/Swine/Schleswig-Holstein/1/93 | 1993 | H1N1 |
| U72667 | A/Swine/England/195852/92 | 1992 | H1N1 |
| Z46435 | A/swine/Schleswig-Holstein/1/92 | 1992 | H1N1 |
| Z46434 | A/swine/Germany/8533/91 | 1991 | H1N1 |
| AF091315 | A/Swine/Italy-Virus/671/87 | 1987 | H1N1 |
| AF320062 | A/Swine/Netherlands/1/87 | 1987 | H1N1 |
| AF320065 | A/Swine/Netherlands/386/86 | 1986 | H1N1 |
| AF320056 | A/Swine/France/3614/84 | 1984 | H1N1 |
| AJ344015 | A/swine/Finistere/2899/82 | 1982 | H1N1 |

To generate the above table, an 11 nucleotide probe of HA in H5N1 with polymorphisms G746A and A748C was used to identify donor sequences that would generate the HA polymorphism G228S in H5N1 circulating in migratory birds in Europe. The above sequences at the Loa Alamos Influenza Database contain an exact match with the probe sequence listed above. All of the isolates were H1 and all but one were H1N1.

The donor sequence was found in isolates from swine in Europe from 1982 to 2001. Because 2001 was the most recent date for European swine isolates in the database, it was likely that the G228S polymorphism-containing parental viral strain (donor) sequences were also currently in European swine. In 2005, H5N1 entered eastern Europe via migratory birds from Siberia. These sequences will migrate through the Middle East and into Africa for the winter. In the spring, migratory birds will carry H5N1 sequences through western Europe, allowing for dual infections in swine and the acquisition of G228S by H5N1 via gene transfer events as described herein.

The serine at position 228 has been found in human H3 isolates and has increased affinity for human receptors. The acquisition of G228S by H5N1 will likely increase the efficiency of H5N1 infections in humans. While the H1N1 contains a G228S polymorphism, the polymorphism was identified in the H3N2 viral strain to impart enhanced affinity for human receptors. Interestingly, H3N2 has an S at position 227, so wild birds with viral strain sequences that have not acquired S227N would match H3 at both positions 227 and 228 (both would be S and both are in the receptor binding domain). Thus, there would be the potential for both S227N, G228S as well as S227, G228S in newly-arising progeny strains of virus.

A similar search was performed for the HA Q226L polymorphism, with results shown in Table 20C below:

TABLE 20C
Search Results for Polymorphism Q226L
Q226L (S227N)
A741T A742C
Probe: CtcAaTGGAAG

| | | | |
|---|---|---|---|
| AY139072 | B/Oman/16296/2001 | NA | 2001 |
| AY582011 | B/Nanchang/1/00 | NA | 2000 |
| AY139056 | B/Bangkok/34/99 | NA | 1999 |
| AY139055 | B/Bangkok/54/99 | NA | 1999 |
| AB036868 | B/Nagoya/20/99 | NA | 1999 |
| AB036871 | B/Chiba/447/98 | NA | 1998 |
| ISD3BEFFD0D | B/HENAN/74/98 | NA | 1998 |
| AY582006 | B/Nanchang/6/98 | NA | 1998 |
| AY582009 | B/Shiga/51/98 | NA | 1998 |
| ISD3BE70252 | B/SHIGA/51/98 | NA | 1998 |
| ISD3BEFFD56 | B/SIRIRAJ/09/98 | NA | 1998 |
| ISDN13229 | B/WUHAN/49/98 | NA | 1998 |
| ISD3BEFF855 | B/BEIJING/09/97 | NA | 1997 |
| ISD3BEFF645 | B/BEIJING/22/97 | NA | 1997 |
| AY139078 | B/Beijing/243/97 | NA | 1997 |
| AY139052 | B/Guangzhou/7/97 | NA | 1997 |
| AY582002 | B/Henan/22/97 | NA | 1997 |
| AY582005 | B/Nanchang/15/97 | NA | 1997 |
| AY582003 | B/Nanchang/2/97 | NA | 1997 |
| AY582004 | B/Nanchang/4/97 | NA | 1997 |
| AY139054 | B/Osaka/547/97 | NA | 1997 |
| ISD3BEFFAE0 | B/SHANGHAI/04/97 | NA | 1997 |
| AY139050 | B/Hong Kong/70/96 | NA | 1996 |
| AF129921 | B/Memphis/20/96 | NA | 1996 |
| AY139051 | B/Sichuan/281/96 | NA | 1996 |
| AY581995 | B/Nanchang/3/95 | NA | 1995 |
| AY139079 | B/Guangdong/5/94 | NA | 1994 |
| AY581993 | B/Nanchang/560/94 | NA | 1994 |
| AY581994 | B/Nanchang/630/94 | NA | 1994 |
| AY581987 | B/Nashville/45/91 | NA | 1991 |
| AF129908 | B/Panama/45/90 | NA | 1990 |

Above, an 11 nucleotide probe of HA H5N1 that has already acquired S227N was used to identify donor sequences containing A741T and A742C to create Q226L. Q226L is a polymorphism in the receptor binding domain that increases binding affinity for human receptors.

The parental (donor) viral strain sequences were in NA from influenza β isolates from 1990 to 2001. The likelihood of this recombination generating Q226L was reduced for several reasons. The parental (donor) viral strain sequence had evolved away from that of currently circulating influenza B. It was not found in the influenza B sequences from isolates from 2002 to 2005, so current circulation levels are low. In addition, current infections of humans by H5N1 are low, reducing the likelihood of a dual infection between H5N1 with S227N and B influenza. In addition, the HA parental (donor) viral strain sequences were in NA. Accordingly, the Q226L polymorphism is not predicted to be positioned for genetic transfer events in the same manner as, e.g., the G228S polymorphism. Thus, the results of the above query for the Q226L polymorphism demonstrate that parental (donor) viral strain polymorphic sequences of potential impact are not always readily available, underscoring the predictive value of the preceding approach in identification of, e.g., the S227N and G228S gene transfer events as likely to occur.

The HA E190D polymorphism was also assessed for likelihood of emerging in a progeny strain via gene transfer. Potential parental (donor) sequences for E190D are shown below in Table 20D.

TABLE 20D
Search Results for Polymorphism E190D
G634T
E190D
TGCGGCAGAt

| | | | |
|---|---|---|---|
| AF202248 | A/gull/Italy/692-2/93 | 1993 | H7N2 |
| AF202242 | A/psittacine/Italy/1/91 | 1991 | H7N2 |
| AF202239 | A/chicken/Ireland/1733/89 | 1989 | H7N7 |
| U20465 | A/duck/Heinersdorf/S495/6/86 | 1986 | H7N7 |
| AF202236 | A/chicken/England/71/82 | 1982 | H7N1 |
| AF202250 | A/macaw/England/626/80 | 1980 | H7N7 |
| AF149295 | A/African Starling/England-Q/938/79 | 1979 | H7N1 |
| AF202232 | A/African starling/England-Q/983/79 | 1979 | H7N1 |
| U20459 | A/chicken/Leipzig/79 | 1979 | H7N7 |
| L43913 | A/goose/Leipzig/137/79 | 1979 | H7N7 |
| L43914 | A/goose/Leipzig/187/79 | 1979 | H7N7 |
| L43915 | A/goose/Leipzig/192/79 | 1979 | H7N7 |
| AF202245 | A/turkey/England/192-328/79 | 1979 | H7N3 |
| AF202235 | A/turkey/Israel/Ramon/79 | 1979 | H7N2 |
| AF202247 | A/turkey/England/647/77 | 1977 | H7N7 |
| AF202252 | A/parrot/Northern Ireland/VF-73-67/73 | 1973 | H7N1 |
| AF202238 | A/turkey/England/63 | 1963 | H7N3 |
| U20462 | A/turkey/England/63 | 1963 | H7N3 |

The isolates containing the donor sequence for G634T, which creates E190D are listed above. All donor sequences were found in H7 in avian isolates from Europe and Israel between 1963 and 1993. The absence of donor sequences from recent H7 isolates reduced the likelihood of the recent Qinghai strain of H5N1 acquiring G634T. However, past H7 outbreaks in Europe were common, dating back to the Rostock “fowl plague” H7N1 outbreak in 1934. An H7 outbreak occurred as recently as the 2003 H7N7 outbreak in the Netherlands, raising the likelihood that H7 donor sequences exist in northern Europe but were not represented in the database.

TABLE 20F
Search Results for Polymorphism E190D IN PB2 Parental (Donor)
Sequence
G634T
E190D
tCAGACAAAG
PB2

| | | |
|---|---|---|
| AJ781210 | B/England/23/04 | 2004 |
| AJ781208 | B/Oslo/71/04 | 2004 |
| AJ781211 | B/Bangkok/460/03 | 2003 |
| AJ781200 | B/Barcelona/215/03 | 2003 |
| AJ781204 | B/Bucharest/795/03 | 2003 |
| AJ781209 | B/Cheju/303/03 | 2003 |
| AJ781201 | B/Geneva/5079/03 | 2003 |
| AJ781199 | B/Israel/95/03 | 2003 |
| AJ781212 | B/Jiangsu/10/03 | 2003 |
| AY582071 | B/Memphis/13/03 | 2003 |
| AJ781198 | B/Moscow/3/03 | 2003 |
| AJ781203 | B/Hong Kong/293/02 | 2002 |
| AY582070 | B/Los Angeles/1/02 | 2002 |
| AJ781207 | B/Shanghai/361/02 | 2002 |
| AJ781194 | B/Tehran/80/02 | 2002 |
| AJ781196 | B/Trieste/28/02 | 2002 |
| AJ781206 | B/Ulan Ude/4/02 | 2002 |
| AJ781197 | B/Hong Kong/330/01 | 2001 |
| AY504607 | B/Hong Kong/330/2001 | 2001 |
| AY504615 | B/Hong Kong/330/2001 (egg adapted) | 2001 |
| AJ781202 | B/Hong Kong/692/01 | 2001 |
| AY582067 | B/Maryland/1/01 | 2001 |
| AY582068 | B/Nebraska/1/01 | 2001 |
| AY582069 | B/Nebraska/2/01 | 2001 |
| AJ781205 | B/Hong Kong/557/00 | 2000 |
| AY504599 | B/Victoria/504/2000 | 2000 |
| AJ781192 | B/Sichuan/379/99 | 1999 |
| ISDNCHB017 | B/Vienna/1/99 (Vero 1 isolate) | 1999 |
| AY687394 | B/Beijing/76/98 | 1998 |
| AF101976 | B/Chiba/447/98 | 1998 |
| AY582066 | B/Nanchang/6/98 | 1998 |
| AF101985 | B/Shiga/51/98 | 1998 |
| AF101986 | B/Shiga/T30/98 | 1998 |
| AF101990 | B/Yamanashi/166/98 | 1998 |
| AF101975 | B/Beijing/243/97 | 1997 |
| AF101980 | B/Henan/22/97 | 1997 |
| AY260943 | B/Memphis/12/97 | 1997 |
| AY260950 | B/Memphis/12/97 | 1997 |
| AY582065 | B/Nanchang/2/97 | 1997 |
| AJ781193 | B/Shandong/7/97 | 1997 |
| AF484967 | B/Shangdong/7/97 | 1997 |
| AY582064 | B/Nanchang/6/96 | 1996 |
| AF101978 | B/Guangdong/5/94 | 1994 |
| AF170572 | B/Harbin/7/94 | 1994 |
| AF101979 | B/Harbin/7/94 | 1994 |
| AY582062 | B/Nanchang/560/94 | 1994 |
| AY582063 | B/Nanchang/630/94 | 1994 |
| AJ781195 | B/Beijing/184/93 | 1993 |
| AF101974 | B/Beijing/184/93 | 1993 |
| AY582061 | B/Memphis/5/93 | 1993 |
| AF101983 | B/Mie/1/93 | 1993 |
| AY582060 | B/Houston/1/91 | 1991 |
| AF005737 | B/Panama/45/90 | 1990 |
| AY582059 | B/Memphis/3/89 | 1989 |
| AF101973 | B/Aichi/5/88 | 1988 |
| AF101989 | B/Yamagata/16/88 | 1988 |
| AF101988 | B/Victoria/2/87 | 1987 |
| AF101981 | B/Ibaraki/2/85 | 1985 |
| AF101984 | B/Norway/1/84 | 1984 |
| AF101987 | B/Singapore/222/79 | 1979 |
| ISDNCHB035 | B/Russia/69 (egg isolate) | 1969 |
| M20163 | B/Ann Arbor/1/66 (cold-adapted) | 1966 |
| M20168 | B/Ann Arbor/1/66 (wild-type) | 1966 |

In the above table, a 10 nucleotide probe was used to identify donor (parental) sequences on PB2 of influenza B. The donor sequences were in isolates from around the world collected from 1966 to 2004. The potential for gene transfer of this polymorphism into HA sequence of emerging progeny strains is low, as the parental (donor) sequence is on a distinct gene (PB2, rather than HA) and would require dual infections with humans. Currently, this event is less likely than those presented for, e.g., G228S, but acquisition of G228S and/or E190D would increase the chance of this gene transfer event occurring because more humans would be infected with H5N1.

### Example 5

### Selection Methods for Identification of Parental Viral Strains

The above-described predictive methods can be further applied to enhance prediction of disease-relevant progeny strains of virus through use of in vitro selection methods to identify parental viral strain sequences of enhanced, e.g, molecular, clinical and/or pathological interest. For example, molecular modeling and/or in vitro approaches to assess viral infectivity, propagation, etc. in the presence or absence of specific mutant forms of parental virus are used to identify parental viral strains of heightened clinical interest. In a specific embodiment, parental strain mutations in the HA protein of the influenza virus are examined for alteration of binding affinity via glycan microarray profiling, performed as described in Stevens et al. (J. Mol. Biol. 355: 1143-55). Identification of parental strain mutations of greatest impact upon, e.g., human receptor affinity, allows for prioritization of parental strains of virus to be used in certain methods of the invention.

### Example 6

### Selection Methods for Identification and/or Prioritization of Progeny Viral Strains

The above-described predictive methods are further refined and applied to enhance prediction of disease-relevant progeny strains of virus through use of both in vitro and in vivo selection methods. For example, parental strains of virus, e.g., that are predicted to mix and result in generation of specific progeny strains of virus, are experimentally combined in vitro via co-infection of cell lines of an appropriate host animal, in order to screen for generation of a predicted type of progeny strain (and/or identify the emergence of a specific progeny strain). In a specific application of this selection methodology, the host cell line is a primate cell line, possibly a human cell line, thereby selecting for progeny strains of virus most capable of infection of and/or propagation in mammalian, primate and/or human cells. Host cell lines used for this approach include those derived from: mammals, e.g., humans, dogs, cows, horses, swine, sheep, goats, cats, mice, rabbits, rats, and transgenic non-human animals, or birds, e.g., ducks, chicken, geese, and swans, and transgenic non-human animals.

In another applied selection methodology for progeny strains of virus, parental strains of virus are used to infect a non-human host animal in vivo, thereby simulating co-infection events, and allowing for better prioritization of predicted progeny strains of virus, e.g., to use for vaccine synthesis. Host animals used for this approach include, e.g., mammals, e.g., dogs, cows, horses, swine, sheep, goats, cats, mice, rabbits, rats, or birds, e.g., ducks, chicken, geese, and swans, and transgenic non-human animals Glycan chips are also used for screening various receptor binding domain combinations to ascertain the affinities of such polymorphic domains for various receptors. The receptor binding domain affinities of individual mutations or, e.g., combinations of polymorphisms like SS or NS at positions 227 and 228 on various H5N1 backgrounds, are screened for and, as above, used to enhance prediction/prioritization of progeny strains of virus that are of interest, e.g., for use in vaccine synthesis.

### Example 7

### H5N1 Influenza Polymorphisms of Potential Disease Relevance

Sequence of four representative H5N1 influenza isolates (China A/chicken/Guangdong/174/04; Indonesia A/Dk/Indonesia/MS/2004; Vietnam A/Viet Nam/1203/2004; and Turkey A/Cygnus olor/Astrakhan/Ast05-2-2/2005) was examined for polymorphic residues. Identified influenza polymorphisms of potential impact upon disease are presented in Tables 21A through 21F:

TABLE 21A
HA Polymorphisms
HA Polymorphisms

China (10)
F8L V10I K156R T175I E243G T279A N291S N325S K489R R520G
Indonesia (6)
V10I T279A V497I D503N D509N V549M
Vietnam (10)
T52K A102V N110D D139S S144L K156R A172T R125K K228R T533I
Turkey (8)
I87L A99I K156R N170D S171N Y268N M298I R339G

The above table presents HA polymorphisms co-circulating in 2004/2005 in representative isolates that can cause fatal human infections in China, Indonesia, Vietnam, and Turkey. Because each isolate contained 6 to 10 polymorphisms that were unique or found in only two of the above representative sequences, anti-sera directed against a specific target offers significantly reduced protection against the other targets, These polymorphisms (which can be considered as variant residues with respect to a specific antisera-strain pairing examined) reduce or eliminate the efficacy of cross-reacting antibodies and allow H5N1 strains possessing variant residues to escape immune recognition. The number of polymorphisms identified among the four representative sequences examined highlight the need to identify/predict new and evolving targets on a seasonal basis for vaccine development.

TABLE 21B
NA Polymorphisms

China (5)
N53K F54V Y79H T127X E361G
Indonesia (10)
R43H A45S P47S F54P S75R Y79H V318M P320L D431N G434S
Vietnam (9)
I17T Q38H R43H A45S T64K S74N Y233H D262N P320S
Turkey (5)
N53K K90R K332R E361G G434S

The above table contains NA polymorphisms co-circulating in 2004/2005 in representative isolates of H5N1 that can cause fatal human infections in China, Indonesia, Vietnam, and Turkey. Because each isolate contained 5 to 10 polymorphisms that were unique or found in only two of the above representative sequences, anti-sera directed against a specific target offers significantly reduced protection against the other targets. As in Table 21A, these polymorphisms (which can be considered as variant residues with respect to a specific antisera-strain pairing examined) reduce or eliminate the efficacy of cross-reacting antibodies and allow H5N1 strains possessing variant residues to escape immune recognition. The number of polymorphisms identified among the four representative sequences examined highlight the need to identify/predict new and evolving targets on a seasonal basis for vaccine development.

TABLE 21C
M2 Polymorphisms

China (2)
V26A A66E
Indonesia (1)
C18Y
Vietnam (3)
L25I S31N S64A
Turkey (3)
C18Y A66G S82N

The above table contains M2 polymorphisms co-circulating in 2004/2005 in representative isolates of H5N1 that can cause fatal human infections in China, Indonesia, Vietnam, and Turkey. Each isolate had 1 to 3 polymorphism that were unique, or found in only two of the above representative sequences of the 97 amino acid protein.

TABLE 21D
PB2 Polymorphisms

China (5)
L10I I67V D309N K312R F610L
Indonesia (4)
Q287R I291T R368Q R389K
Vietnam (4)
T105A T339K E391Q E627K
Turkey (9)
A108T I147T R368Q D390N I452T M483V T521N E627K V649I

The above table contains PB2 polymorphisms co-circulating in 2004/2005 in representative isolates of H5N1 that can cause fatal human infections in China, Indonesia, Vietnam, and Turkey. Each isolate had 4 to 9 polymorphisms that were unique, or found in only two of the above representative sequences.

TABLE 21E
PB1 Polymorphisms

China (1)
I682V
Indonesia (3)
S80C K215R T401A
Vietnam (0)
Turkey (4)
K353R L364V S654G Q756P

The above table contains M2 polymorphisms co-circulating in 2004/2005 in representative isolates of H5N1 that can cause fatal human infections in China, Indonesia, Vietnam, and Turkey. Each isolate had 0 to 4 polymorphisms that were unique, or found in only two of the above representative sequences.

TABLE 21F
PA Polymorphisms

China (8)
S59G E101N T110I K204R I459T V555I A669V A712T
Indonesia (4)
V450I L469M F520Y P653S
Vietnam (2)
K123E S421I
Turkey (4)
S184G A337T K367R P653S

The above table contains PA polymorphisms co-circulating in 2004/2005 in representative isolates of H5N1 that can cause fatal human infections in China, Indonesia, Vietnam, and Turkey. Each isolate had 2 to 8 polymorphisms that were unique, or found in only two of the above representative sequences.

TABLE 21G
NP Polymorphisms

China (0)
Indonesia (1)
R400K
Vietnam (0)
Turkey (4)
V35I Y111H R384K V408I

The above list contains NP polymorphisms co-circulating in 2004/2005 in representative isolates of H5N1 that can cause fatal human infections in China, Indonesia, Vietnam, and Turkey. Each isolate had 0 to 4 polymorphisms that were unique, or found in only two of the above representative sequences.

TABLE 21H
NS1 Polymorphisms

China (4)
S3P V106M S200G P208T
Indonesia (7)
S7L A81T I123V G165D S200G L209F V225I
Vietnam (5)
E71G E75K S191T S200N 216TR*
Turkey (10)
A107T F133Y G165N V190I T193I S201I D204G N212D S223P E224K
*TR = Truncated

The above list contains NS1 polymorphisms co-circulating in 2004/2005 in representative isolates of H5N1 that can cause fatal human infections in China, Indonesia, Vietnam, and Turkey. Each isolate had 4 to 10 polymorphisms that were unique, or found in only two of the above representative sequences.

TABLE 21I
NS2 Polymorphisms

China (2)
S3P F55L
Indonesia (3)
S7L V14M I83V
Vietnam (2)
A48T I83V
Turkey (4)
V14M V49L M52V A115T

The above list contains NS2 polymorphisms co-circulating in 2004/2005 in representative isolates of H5N1 that can cause fatal human infections in China, Indonesia, Vietnam, and Turkey. Each isolate had 2 to 4 polymorphisms that were unique, or found in only two of the above representative sequences.

TABLE 21J
M1 Polymorphisms

China (2)
K27R M248I
Indonesia (0)
Vietnam (1)
V206I
Turkey (2)
I59M M248I

The above list contains M1 polymorphisms co-circulating in 2004/2005 in representative isolates of H5N1 that can cause fatal human infections in China, Indonesia, Vietnam, and Turkey. Each isolate had 0 to 2 polymorphism that was unique, or found in only two of the above representative sequences.

### Example 8

### Evolution of Influenza a by Recombination with Swine Isolates

### Methods

Sequences were aligned and numbered using BLAST. Polymorphisms were defined by the seven swine sequences being tested and matching sequences. Identity was defined by three consecutive matching polymorphisms. Matching haplotypes were identified by screening 1111 human and 25 swine PB1 sequences. Accession numbers, names, and abbreviations were listed.

The looming influenza pandemic has focused attention on the rapid evolution of H5N1 and other human and avian serotypes. The rapid evolution of influenza viruses has created a challenge in vaccine development.

To investigate the role that domestic animals might play in harboring influenza isolates distinct from those currently circulating in, e.g., humans and migratory birds, swing influenza isolate sequences were examined. Accordingly, swine influenza isolates from 2003 and 2004 were identified that had acquired a human flu gene, PB1. Sequence analysis of the eight flu gene segments was performed and revealed large portions of two genes, PB2 and PA, as identical matches with 1977 isolates, while additional regions were exact matches with 1998 and 2002 isolates. Such matched sequences across decades of time demonstrated the existence of homologous recombination contributing tracts of earlier genomes to present flu genomes. These data discounted the role of point mutations and highlighted the role of homologous recombination in the evolution of these genes.

Moreover, the human PB1 gene was observed to have evolved more slowly in swine and, accordingly, swine appear to have acted as a reservoir for acquisition of polymorphsims via recombination in human seasonal flu. Certain PB1 sequences were initially present in human flu isolates from the mid 1990's. After such sequences had disappeared from the human population for several years, the sequences reappeared in the dominant 2004/2005 human PB1 haplotype via homologous recombination.

H1N1 and H1N2 swine isolates in Canada contained a constellation of seven swine genes and one human gene, PB1. The eight gene segments of seven recent isolates were compared to influenza sequences at the Loa Alamos influenza database. This analysis identified extensive regions of identity with earlier isolates.

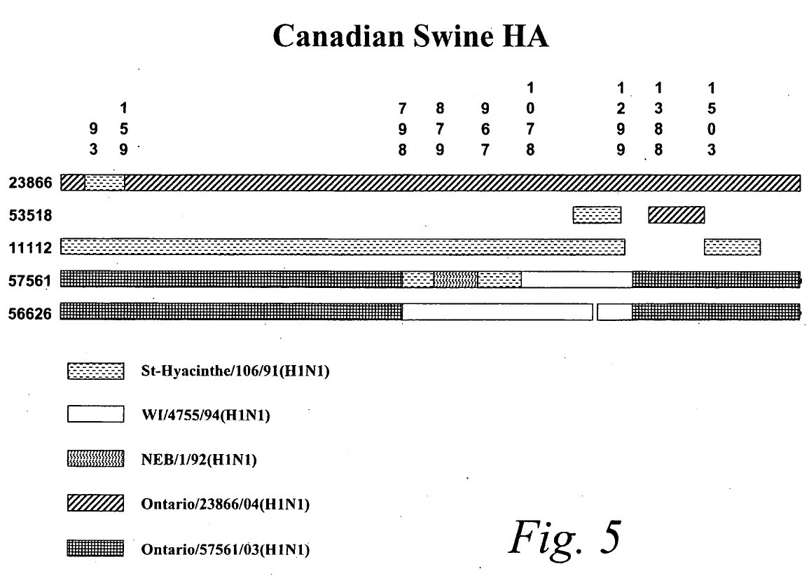

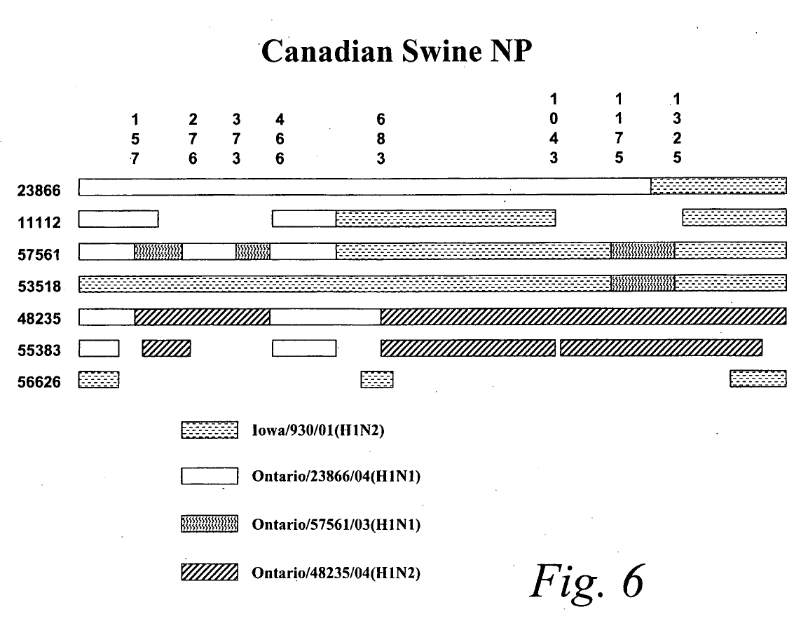

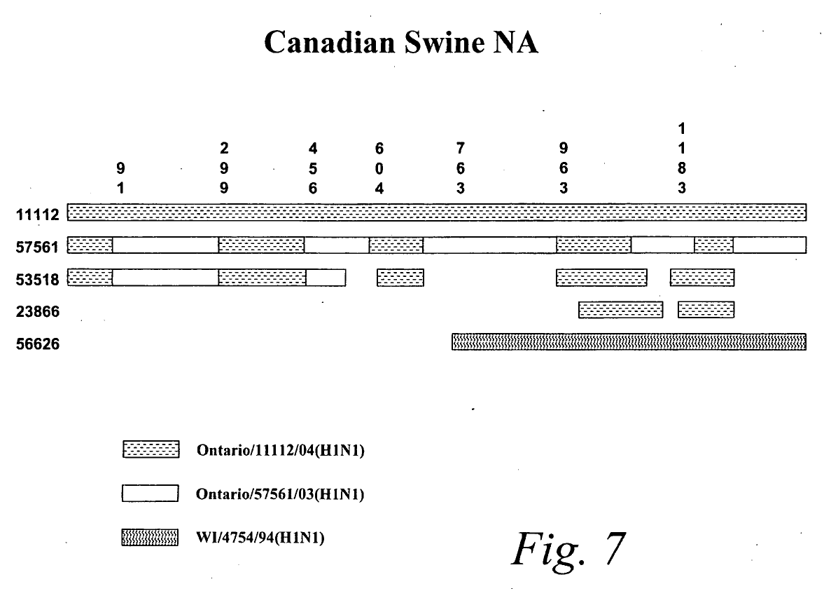

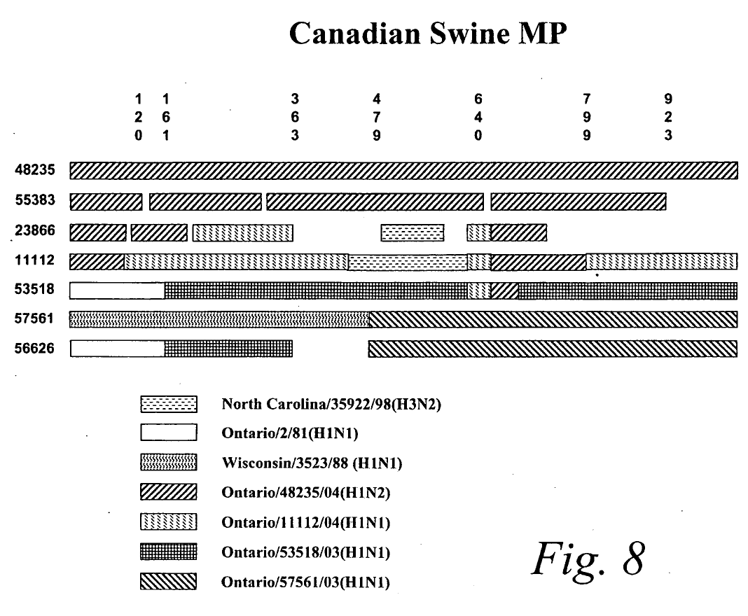

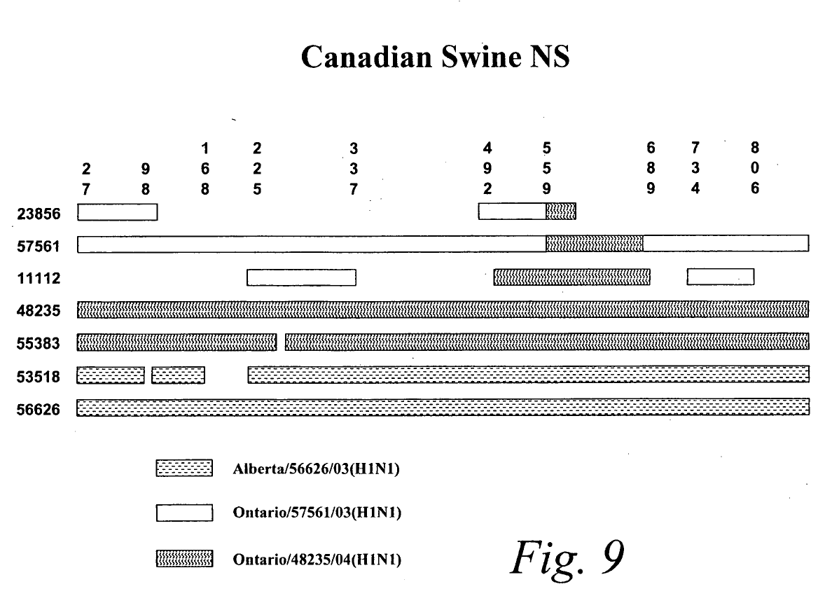

The eight influenza gene segments of the seven swine isolates were analyzed for regions of sequence that exactly matched other isolates (FIGS. 2-9). Regions of identity observed in the PB2 gene are presented in FIG. 2. The regions were defined by an exact match of three consecutive polymorphisms, defined by the seven swine sequences. Five of the swine isolates, SW/Ont/23866/04(H1N1), SW/Ont/57561/03(H1N1), SW/Alb56626/03(H1N1), SW/Ont/48235/04(H1N2), SW/Ont/55383/04(H1N2) had regions of homology with SW/Tennessee/24/77(H1N1). All five matched the 1977 isolate between positions 1006 and 1326. However, outside of this core region all sequences diverged from each other. SW/Ont/57561 and SW/Ont/56626 possessed flanking regions which matched SW/North Carolina/35922/98(H1N1) and an upstream region that matched SW/Ont/11112/04. However, the two closely related sequences then diverged from each other. SW/Alb/56626 matched SW/Ont/53518. However, SW/Ont/53518 did not have matching regions for the 1977 isolate. Instead, the remainder of the downstream region matched SW/Korea/CT02/02(H1N1). SW/Ont/23866 had a larger region matching the 1977 isolate, which extended to position 755. Upstream, the sequence matched Sw/Ont/11112. Downstream from position 1594, there was also a match between SW/Ont/11112 and SW/Ont/23866. The region in SW/Ont/11112 that did not match SW/Ont/23866 matched the 1998 North Carolina isolate. SW/Ont/48235 and SW/Ont/55383 had the longest region of homology with the 1977 isolate, which began at position 204 and extended to position 1931 for SWOnt//55383. SW/Ont/48235 was identical for a slightly shorter region. Both isolates then matched other Canadian swine isolates, but SW/Ont/55383 diverged to a unique sequence at position 2224.

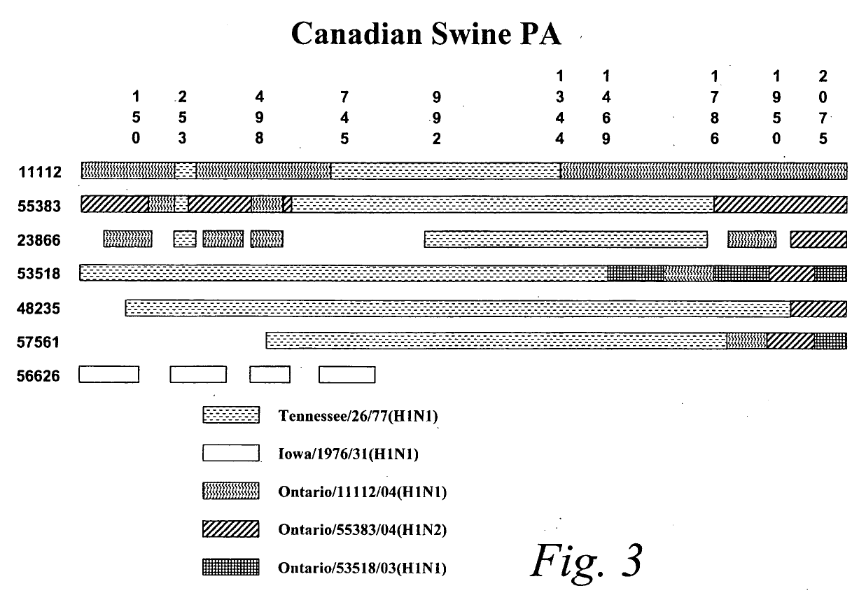

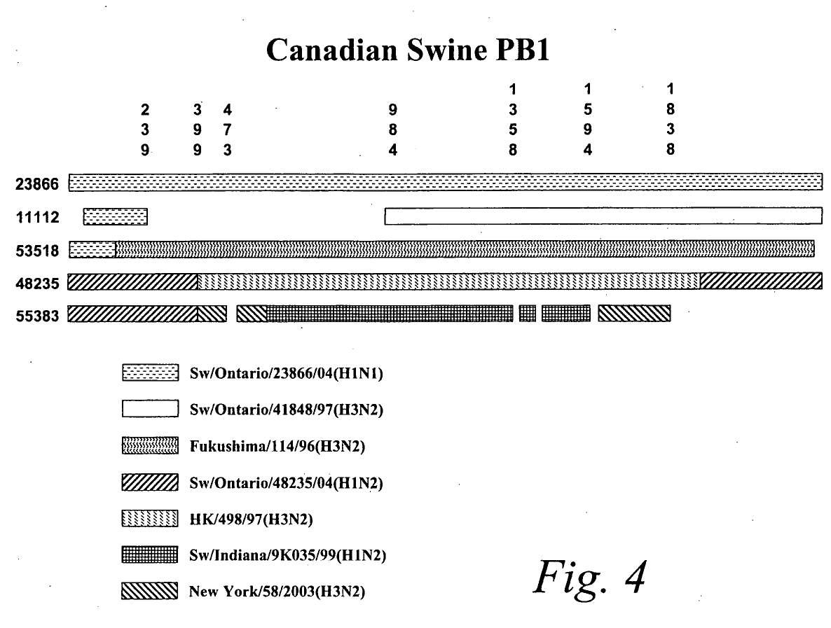

Regions of identity were also found in the PA gene (refer to FIG. 3). In these genes, six of the seven Canadian swine isolates had large regions of identity with another 1977 swine isolate, SW/Tennessee/26/77(H1N1). All six were identical to the 1977 isolate between positions 992 and 1344. However, all six had extended regions of identity beyond this common region. The longest region of identity was in SW/Ont/48235, which extended from position 150 to 1950. Outside of the regions of identity with the 1977 isolate, the Canadian isolates had various regions of homology with each other. The seventh Canada isolate, SW/Ont/56626 had extended segments of identity with a 1931 isolate, SW/Iowa/1975/31(H1N1). The remaining six gene segments (refer to FIGS. 4-9) displayed regions of identity with more recent swine isolates, with the exception of PB1, which showed regions of identity with human isolates.

The PB1 gene was most closely related to human sequences of the mid 1990's, indicating that human sequences had evolved away from the swine sequences. To monitor this evolution, three 120 bp regions of the swine sequences were used to probe 1111 human sequences in the Los Alamos database. The distribution of haplotypes found in thirty or more human sequences are listed in Tables 22A-22C. Table 22A has the result of a probe representing the sequence of SW/Ont/55383 and SW/Ont/48235 at positions 1345-1464. This sequence was found in swine isolates in Asia and North America from 1997 through 2004.

TABLE 22A
Evolution of Human PB1 Polymorphisms: Probe Representing Positions
1345-1464 of SW/Ont/55383 and SW/Ont/48235

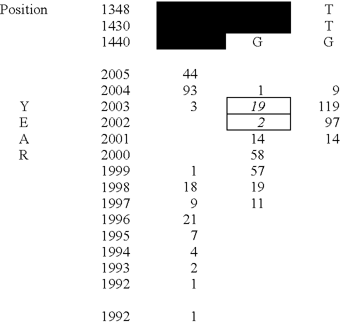

Table 22A (above) presents the major haplotypes at positions 1345-1464 of SW/ON/48235 and SW/ON/55383. Additional matching swine sequences were SW/HK/411/02(H3N2), SW/HK/74/02(H3N2), SW/KO/CY02/02(H1N2), SW/MN/555551/00(H1N2), SW/IA/533/99(H3N2), SW/LA/569/99(H3N2), SW/MN/593/99(H3N2), SW/HK/2429/98(H3N2), SWIowa/8548-1/98(H3N2), SW/MN/9088-2/98(H3N2), SW/NE/209/98(H3N2), SW/TX/4199-2/98(H3N2), SW/SZ/110/97(H3N2), SW/SZ/115/97(H3N2), SW/SZ/119/97(H3N2) and SW/SZ/120/97(H3N2). Positions with white background matched the swine sequence. The number of human H3N2 isolates with matching haplotype are also listed, while human H1N2 matches are boxed and italicized.

As expected, the sequence (“C-C-A” at positions 1348, 1430 and 1440) was found in human isolates from 1992 to 1999 with peak levels in 1996-1998. The sequence was not found in human isolates from 2000 to 2002. During that time, the sequence with A1440G was the dominant haploype in 1999 and 2000, which was then replaced with a hapoltype containing C1348T, C1430T, A1440G, which was dominant in 2002 and 2003. The swine sequence then reappeared in the human population as the dominant haplotype in 2004 and 2005. The reappearance of three polymorphisms that matched the earlier swine sequence evidenced acquisition of these polymorphisms had occurred via recombination with a swine reservoir. These sequences were found in swine isolates identified during the time that the haplotype was not present in the human isolates.

Table 22B shows an upstream region 1225 to 1344 of the SW/Ont/48235 sequences which also matched swine sequences from 1997 to 2004.

TABLE 22B
Probe Corresponding to Upstream Region 1225-1344 of SW/Ont/48235

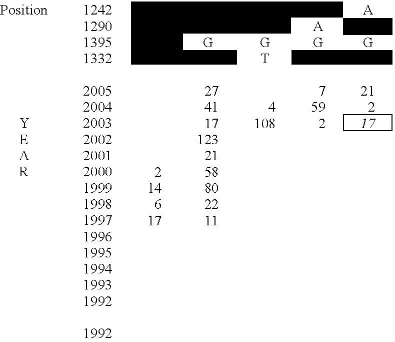

Table 22B (above) presents the major haplotypes at positions 1225-1344.of SW/ON/48235 and SW/ON/55383. Positions with white background matched the swine sequence. The number of human H3N2 isolates with matching haplotype are also listed, while human H1N2 matches are boxed and italicized.

As expected, the sequence (“GGAC” at positions 1242, 1290, 1395 and 1332) was found in human influenza isolates from 1997 to 2000, but the sequence did not reappear in human isolates.

Table 22C shows the downstream region 1765 to 1884 of the SW/Ont/48235 sequence which also matched swine sequences from 1997 to 2004.

TABLE 22C
Probe Corresponding to Downstream Region 1765-1884 of SW/Ont/48235

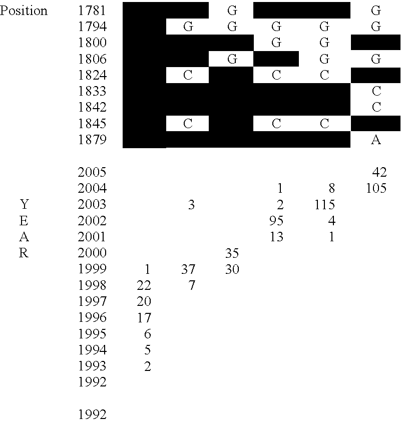

Table 22C (above) presents the major haplotypes at positions 1765-1824.of SW/ON/48235 and SW/ON/55383. Positions with white background matched the swine sequence. The number of human H3N2 isolates with matching haplotype are also listed.

The haplotype (“A-A-A-A-T-T-T-T-G” at positions 1781, 1794, 1800, 1806, 1824, 1833, 1842, 1845 and 1879) was found in human isolates from 1993 to 1999 and was the dominant human haplotype from 1996 to 1998 and did not reappear, demonstrating that the first region was preferentially selected for reappearance in the human population, indicating reappearance of the portion of the swine sequence by recombination of human sequence with swine sequences that were maintained from 1997 to 2004 in a separate (e.g., swine) reservoir.

The sequences in the upstream and downstream regions also showed abrupt changes that involved the acquisition of new polymorphisms, as well as loss of recently acquired polymorphisms. The most dramatic example observed was in the change in the downstream dominant haplotype from 2003 to 2004. Four polymorphisms (A1761G, T1833C, T1842C, and G1879A) were acquired and three more were lost (A1800G, T1824C, T1845C). The seven changes in a single year within 98 bp of sequence could most readily be explained by recombination.

The H1 swine sequences evolved at a slower rate than human sequences. The PB2 sequences evidenced this slower change. Five of the seven swine isolates had regions of identity with a 1977 isolate, SW/TN/24, demonstrating that the polymerase could faithfully copy these segments for over 25 years. This observed high degree of fidelity was at odds with current thinking on influenza evolution, which invokes random mutation to explain the genetic drift that creates annual changes in influenza genes. The slower rate of evolution in the swine sequences provided an opportunity to discern another mechanism of evolution, homologous evolution.

Recombination could be readily seen in all eight gene segments, but was striking in the PB2 gene. Five of the seven sequences shared identity with the 1977 isolate between positions 1006 and 1326. Although this region was faithfully copied in five of the isolates, the other two shared identity with an isolate from 1998 or 2002. Moreover, the region outside of the common 1977 region in the five isolates was differentially conserved. Two of the isolates, SW/ON/48235 and SW/ON/55383 retained identity with the 1977 isolate for most of the gene, over 1600 bp. The absolute conservation of most of the gene for over 25 years was inconsistent with a polymerase causing annual genetic drift attributable to copy errors and the absence of a proof reading function.

The PA gene showed a higher level of absolute fidelity copying the PA gene of another isolate from 1977, SW/TN/26. Six of the seven swine isolates showed fidelity with the 1977 isolate, while the seventh isolate, SW/AL/56626, had regions of identity with a 1931 isolate. Although six of the isolates shared a region of identity between positions 992 and 1344, large staggered regions of identity with the 1977 isolate were present in the recent isolates, which indicated that change was not due to random mutations, and the regions outside of the 1977 segments were acquired via homologous recombination.

Homologous recombination can involve a single crossover or multiple crossover events. The PB2 gene was identical between SW/ON/56626 and SW/ON/53518 for the first 550 bp. SW/ON/53518 matched SWKO/CY02 for the remainder of the gene. SW/AL/56626 matched SW/ON/57561 between 550 and 1594. However, the matching region revealed identities with earlier isolates. The two ends of this region matched North Carolina/35922/98(H1N1), which was also present in SW/ON/11112. However, SW/ON/57561 and SW/AL/56626 had sequence of the 1977 isolate nested in the middle of this region. The nesting was most readily explained by the acquisition of the 1977 sequence after the 1998 sequence. However, the acquisition was possible because the more contemporary isolates retained the older sequence and could act as donors. Thus, the swine genes could act as reservoirs of older genes that could be acquired by more recent isolates via recombination.

The acquisition of older sequences from a swine reservoir could also be demonstrated in the human PB1 gene. This gene was most closely related to human PB1 genes circulating in the mid-1990's. The presence of these sequences in 2003/2004 swine isolates again demonstrated that these sequences evolved more slowly in swine and could be acquired by human sequences via recombination. This was demonstrated by the disappearance of the mid 90's polymorphisms and the reappearance of these polymorphisms in 2004. The simultaneous reappearance of the three polymorphisms indicated they were acquired from a non-human reservoir, such as swine. Thus, swine could act as a reservoir of older sequences for acquisition of human influenza genes.

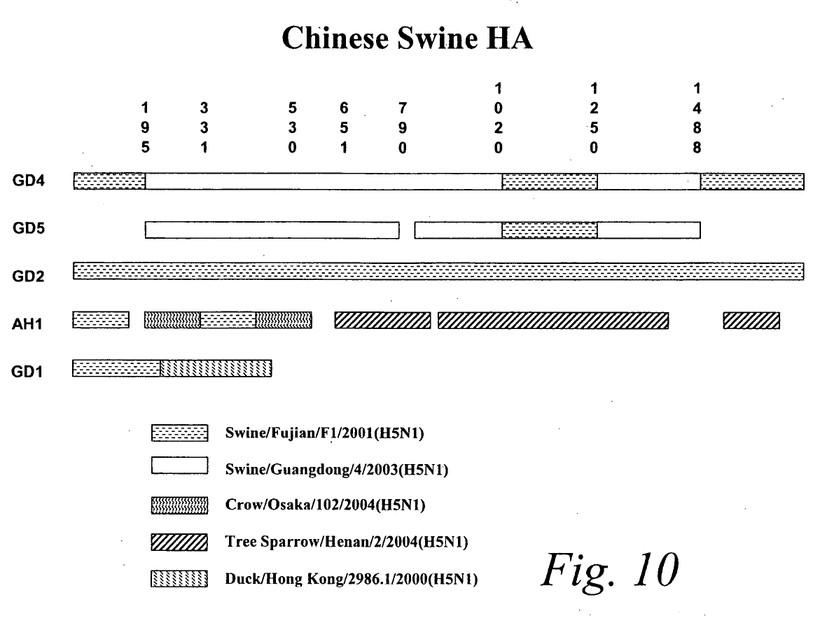

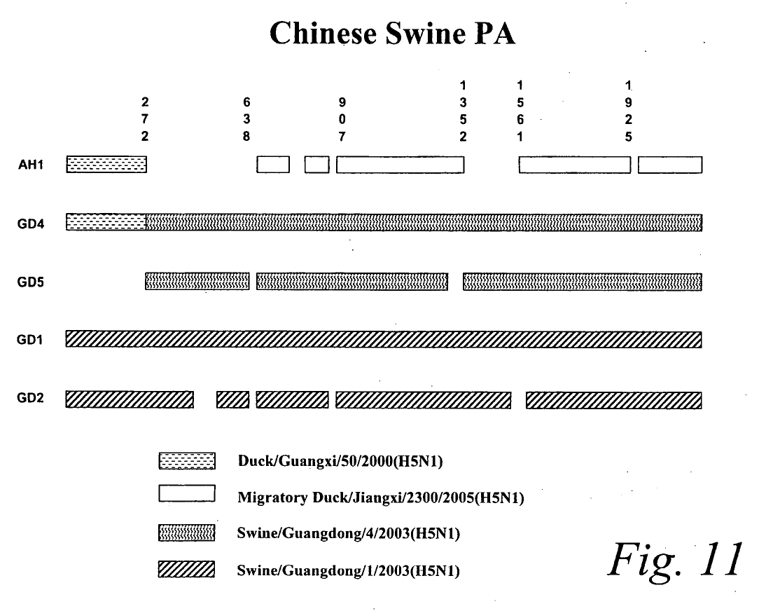

Regions of sequence identity were also observed for Chinese swine influenza isolates in the HA and PA genes (refer to FIGS. 10 and 11). Specifically, regions of sequence identity in HA were observed between Chinese swine and the isolates Swine/Fujian/F1/2001 (H5N1), Swine/Guangdong/4/2003(H5N1), Crow/Osaka/102/2004(H5N1), Tree Sparrow/Henan/2/2004(H5N1) and Duck/Hong Kong/2986.1/2000(H5N1). Regions of sequence identity in PA were observed between the Chinese swine isolates and the isolates Duck/Guangxi/50/2000(H5N1), Migratory Duck/Jiangxi/2300/2005(H5N1), Swine/Guangdong/4/2003(H5N1) and Swine/Guangdong/1/2003(H5N1).

These data showed that swine sequences evolved more slowly and that older sequences in, e.g., swine, could act as a sequence reservoir that could be drawn upon via homologous recombination of swine isolate and human isolate sequences. The acquisition of these sequences via recombination allowed for tracing of origins of sequences and prediction of new acquisitions from prior sequences.

Homologous recombination could be found several times within a gene, and this mechanism could generate single nucleotide polymorphisms when the parental sequences were highly homologous. Multiple recombinations explained the origins of the 1918 pandemic strain, which involved H1N1 human and swine sequences.

Thus, recombination plays an important role in influenza evolution and can generate both drifts and shifts in a predictable manner.

These observations challenged the basic tenets of influenza genetics and provided a method for predicting the changes in seasonal and pandemic influenza, as well as other rapidly evolving genomes.

### Example 9

### Additional Probe Sequences Revealed the Recombinant Flow of Influenza Genomic Information Through Host Animals

An H5 isolate in a mallard in British Columbia was obtained from a collection of August 2005 (SEQ ID NO:1). The isolate was observed to possess a C436T polymorphism. Probe sequence upstream of this polymorphism (ATAATTCCTAGGAGt, where lowercase ‘t’ indicates the polymorphic base) was compared against the influenza database and revealed six influenza isolates from North American shore birds (of H5N7, H5N2 and H5N3 strains). When downstream sequence (tTCTTGGTCCAATCATG) was used to probe the influenza database, however, the vast majority of isolates identified were from Asian birds. Thus, these types of polymorphisms could be used to identify the origins of donor sequences.

Two other probes to additional polymorphisms found in the mallard isolate sequence also revealed the flow of sequences in birds via recombinatory events. The C1249T polymorphism was used to query the flu database using probe sequence AACACTCAGTTTGAGGCt, while the T1492C polymorphism was used to query the flu database using probe sequence cGAATGTATGGAAAGTGTA. These queries both revealed the flow of these sequences/polymorphisms through predominantly H5N1 strains of influenza, including certain isolates from the Qinghai Lake Nature Reserve. The C1249T polymorphism linked to a human serotype in North America in the 1980s and 1990s marked a Qinghai strain that caused human cases in at least 3 countries where H5N1 was first reported in 2006. The T1492C polymorphism also had a link to a human isolate, but it was found only in Hong Kong cases from 1997. Since this polymorphism is not found in the database until 2005, its reemergence indicates a reservoir effect for a sequence not well represented in the database.

Additional probe sequences derived from the same mallard isolate sequence revealed the acquisition of a sequence predominantly found in swine isolates of influenza H1N1 and H1N2, yet the same probe sequence was also shared with isolates of H1N1 that dated to 1933. Probe sequences GAAAAtGAAAGAACTt and GAAAAtGAAAGAACTtTGGATTTCCA both revealed the harboring of these probe sequences in predominantly H1 strains of influenza, predating its recent discovery in the British Columbia mallard H5 influenza isolate. The tracking of two polymorphisms in tandem through a lineage that can be traced back to a 1933 strain of influenza revealed the signature of recombination in such a flow of genetic information. In addition, the recent shifting of a haplotype from H1 isolates in swine to an H5 isolate in a mallard, documented herein, is a recombinant event of potential significance to influenza vaccine development. Accordingly, the observation of this jump from H1 to H5 strains sets forth this haplotype as one that can be used as a probe and/or targeted in a future vaccine for influenza in birds or humans.

### REFERENCES

J S Peiris et al. Lancet, February 2004; 363(9409): 617-9.
Ron Fouchier et al. Nature 435, 419-420 (26 May 2005)|doi: 10.1038/435-419a.
Michael T. Osterholm. N. Engl. J. Med., May 2005; 352: 1839-1842.
Y. Guan et al. PNAS, June 2002; 99: 8950-8955.
Tran Tinh Hien et al.N. Engl. J. Med., March 2004; 350: 1179-1188.
US CDC. JAMA, March 2004; 291: 1059-1060.
Y Guan et al. Virology, January 2002; 292(1): 16-23.
Robert G. Webster et al. J. Virol., January 2002; 76: 118-126.
Aleksandr S. Lipatov et al. J. Virol., March 2003; 77: 3816-3823.
R G Webster et al. Microbiol. Rev., March 1992; 56: 152-179.
Katharine M. Sturm-Ramirez et al. J. Virol., May 2004; 78: 4892-4901.
Li K S et al. Nature. 2004 Jul. 8; 430(6996):209-13.
Y. Guan et al. Proc Natl Acad Sci USA. May 2004; 101: 8156-8161.
Choi Y K et al. Virology. 2005 Feb. 20; 332(2):529-37.
Peiris J S et al. J. Virol. 2001 October; 75(20):9679-86.
Chen H et al. Proc Natl Acad Sci USA. 2004 Jul. 13; 101(28):10452-7. Epub Jul. 2, 2004.
Kanta Subbarao et al. Science, Vol 279, Issue 5349, 393-396, 16 Jan. 1998 [DOI: 10.1126/science.279.5349.393].
E C Claas et al. Lancet, February 1998; 351(9101): 472-7.
Yi Guan et al. PNAS, August 1999; 96: 9363-9367.
Marra M A et al. Science. 2003 May 30; 300(5624):1399-404. Epub 2003 May 1.
Rota P A et al. Science. 2003 May 30; 300(5624):1394-9. Epub 2003 May 1.
van der Hoek L et al. Nat. Med. 2004 April; 10(4):368-73. Epub 2004 Mar. 21.
Woo P C et al. J. Virol. 2005 January; 79(2):884-95.
S J Lolle et al. Nature, March 2005; 434(7032): 505-9.
Krolikowski K A et al. Plant J. 2003 August; 35(4):501-11.
Hatta, M et al. Science 293: 1840-1842 (2001).
Fouchier, R. A. M. et al. Proc. Natl. Acad. Sci. U.S.A. 101: 1356-1361 (2003).
Govorkova, E. A. et al. J. Virol. 79: 2191-2198 (2005).
Thanawongnuwech, R. et al. Emerg Infect Dis, 11: 699-701 (2005)
Lipatov, A. S. et al. J. Virol. 77: 3816-3823 (2003).
Mase, M. et al. Virology doi:10.1016/j.virol. 2005 May 10.
Chen, H. et al. Nature 436: 191-192 (2005).
Choi, Y. K. et al. Virology. 332:529-537 (2005).
Gibbs, M. J. et al. Science 293: 1842-1844 (2001).
Worobey, M. et al. Science 296: 211 (2002).
Lai, M. M. Microbiol. Rev. 56: 61-79 (1992).
Worobey, M & Holmes, E. C J. Gen. Virol. 80: 2535-2543 (1999).
Chare, E. R. et al. J. Gen. Virol. 84: 2691-2703 (2003).

### Equivalents

Those skilled in the art will recognize, or be able to ascertain using no more than routine experimentation, many equivalents to the specific embodiments of the invention described herein. Such equivalents are intended to be encompassed by the following claims.

1. A method of predicting progeny viral strain sequence from sequences of a first parental viral strain and a second parental viral strain, comprising:
identifying a first parental viral strain sequence comprising one or more sequences correlated with a characteristic of the virus;
identifying a second parental viral strain sequence lacking one or more of the one or more sequences of the first parental viral strain; and
predicting progeny viral strain sequences capable of arising from a genetic transfer event comprising replacement of a second parental viral strain sequence with a first parental viral strain sequence
such that a progeny viral strain sequence having a characteristic of the parental viral strain is predicted.

2. The method of claim 1, wherein the viral strains are influenza viruses.

3. (canceled)

4. The method of claim 3, wherein the molecular characteristic is a nucleic acid alteration or amino acid alteration.

5. The method of claim 4, wherein the nucleic acid or amino acid alteration is in an influenza sequence selected from the group consisting of HA, NA, NP, NA, PA, PB1, PB2, M1, M2, NS1, and NS2, or combinations thereof.

6. (canceled)

7. The method of claim 4, wherein the nucleic acid or amino acid alteration is in an influenza HA sequence.

8. (canceled)

9. The method of claim 7, wherein the alteration is in an influenza HA sequence at a residue position(s) selected from the group consisting of 190, 225, 226, 227, 228, and combinations thereof.

10.-11. (canceled)

12. The method of claim 1, wherein the molecular characteristic is selected from the group consisting of viral infectivity, viral antigenicity, viral replication, and viral binding to a host cell receptor.

13. The method of claim 1, wherein the binding of the first parental viral strain to a cellular receptor is altered, as compared to the binding of the second parental viral strain to the cellular receptor.

14. (canceled)

15. The method of claim 13, wherein the host cell receptor is an α2-6-linked sialic acid glycoprotein.

16. The method of claim 1, wherein the first parental viral strain sequence infects a host animal of a population of a first geographic range and the second parental viral strain sequence infects a host animal of a population of a second geographic range.

17. The method of claim 1, wherein at least one of the first or second parental viral strain sequences is isolated from a host animal.

18. (canceled)

19. The method of claim 17, wherein the host animals of the first and second parental viral strains are of different species.

20. The method of claim 17, wherein at least one of the host animals of the first or second parental viral strains is a migratory bird.

21.-33. (canceled)

34. The method of claim 1, wherein the method further comprises producing a therapeutic compound or vaccine to at least one progeny viral strain.

35. The method of claim 34, wherein the method further comprises administration of the therapeutic compound or vaccine to a subject.

36. A sequence identified according to any of the foregoing methods suitable for use in the development of a prognostic compound, diagnostic compound, therapeutic compound, or vaccine.

37.-38. (canceled)

39. A composition comprising an influenza nucleic acid or polypeptide sequence having an alteration as set forth in any of the Tables herein.

40. The composition of claim 39, wherein the nucleic acid or polypeptide sequence is an altered influenza HA sequence.

41. The composition of claim 39, wherein the nucleic acid or polypeptide sequence is an altered influenza NA sequence.

42. The composition of claim 40, wherein the influenza HA sequence comprises an alteration at a residue position(s) selected from the group consisting of 190, 225, 226, 227, 228, and combinations thereof.

43.-51. (canceled)


SEQUENCE LISTING (1 sequence)

SEQ ID NO: 1 - DNA, 1687 bp, Influenza Virus
gtgattgccctcgcaataatcagcattgtcaaaggtgaccaaatctgcattggttatcatgcaaacaattcaacagagcaggttgatacaatcatggagaagaatgtgacggtcacacatgctcaggatatactggaaaaagagcacaatgggaaactttgcagtcttaaaggagtgaggcccctcattctgaaagattgcagtgtagctggatggcttcttggaaacccaatgtgtgatgaattcctgaatgtaccggaatggtcatacatcgttgaaaaagataatccagtcaatggcctgtgctatccaggagacttcaatgattacgaagaactgaagcatttaatgagcagcacaaaccattttgagaaaattcagataattcctaggagttcttggtccaatcatgatgcctcatcaggagtgagttcggcatgcccatacaatggtagatcttcctttttcaggaatgtagtgtggttgatcaagaagaataatgcgtacccaacaataaagaggacctacaacaacaccaatgtagaagaccttttaataatatggggaattcaccaccctaatgatgcagctgagcaaacaaaactctaccagaactcgaacacttatgtgtctgtaggaacatcaacactgaatcagagatcaatcccagaaatagccactagacccaaagtgaacggacaaagtggaagaatggaatttttctggacaatactaaggccgaacgatgcaatcagctttgaaagtaatgggaattttatagctcctgaatatgcgtacaagattgtcaagaaaggagattcagcaatcataagaagtgaattggagtatggtaactgtgacaccaaatgtcagaccccggtgggtgctataaattccagtatgcccttccacaatgttcatcctcttaccattggggagtgccccaagtatgtcaaatcggacaaactggtccttgcaacaggactaagaaacgtaccccaaagagaaacaagaggcctatttggagcaatagcaggattcatagaaggaggatggcaaggaatggttgatggatggtacggataccatcatagcaatgagcagggaagtggatatgctgcagacaaagaatctacccagaaagcaatcgatgggatcaccaataaagtaaactcaatcattgacaaaatgaacactcaattcgaagccgttgggaaagaattcaacaacctagaaaggagaacagaaaatttgaataagaaaatggaagatgggtttttagatgtatggacttacaatgcagaacttcttgtgctcatggaaaatgaaagaactttggatttccatgattcaaatgtcaagaacctatacgataaggtccgactccagctgagagacaatgcaaaagaattgggcaacggatgctttgaattctaccacaagtgtgacaatgaatgcatggaaagtgtgagaaatggaacgtatgactatccgcaatattcagaagaatcaagactgaacagggaggaaatagacggagtcaaattggaatcaatgggcacttatcagatactatcaatctactcaacagtggcgagttccctagcactggcaatcatgatagctggtctatctttttggatgtgctccaatggatcattgcagtgcagaatttgcatctagaatt